SEQUENCE LISTING (51 sequences)

SEQ ID NO: 1 - DNA, 27 bp, Artificial sequence
  Single strand DNA oligonucleotide
ccatcctaatacgactcactatagggc

SEQ ID NO: 2 - DNA, 24 bp, Artificial sequence
  Single strand DNA oligonucleotide
gtagtgatgccatgtaactgaatc

SEQ ID NO: 3 - DNA, 23 bp, Artificial sequence
  Single strand DNA oligonucleotide
actcactatagggctcgagcggc

SEQ ID NO: 4 - DNA, 22 bp, Artificial sequence
  Single strand DNA oligonucleotide
gcatcttagccgtctttcttcg

SEQ ID NO: 5 - DNA, 15 bp, Artificial sequence
  Single strand DNA oligonucleotide
ttttttttttttttt

SEQ ID NO: 6 - DNA, 23 bp, Artificial sequence
  Single strand DNA oligonucleotide
ttcgatcccaagaaggaatcaac

SEQ ID NO: 7 - DNA, 24 bp, Artificial sequence
  Single strand DNA oligonucleotide
gtagtgatgccatgtaactgaatc

SEQ ID NO: 8 - protein, 9 aa, Artificial sequence
  Peptide derived from tryptic digestion of human heparenase
YGPDVGQPR

SEQ ID NO: 9 - DNA, 1721 bp, Homo sapiens
ctagagctttcgactctccgctgcgcggcagctggcggggggagcagccaggtgagcccaagatgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctgggttctccaaagcttcgtaccttggccagaggcttgtctcctgcgtacctgaggtttggtggcaccaagacagacttcctaattttcgatcccaagaaggaatcaacctttgaagagagaagttactggcaatctcaagtcaaccaggatatttgcaaatatggatccatccctcctgatgtggaggagaagttacggttggaatggccctaccaggagcaattgctactccgagaacactaccagaaaaagttcaagaacagcacctactcaagaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatgaacctaacagtttccttaagaaggctgatattttcatcaatgggtcgcagttaggagaagattatattcaattgcataaacttctaagaaagtccaccttcaaaaatgcaaaactctatggtcctgatgttggtcagcctcgaagaaagacggctaagatgctgaagagcttcctgaaggctggtggagaagtgattgattcagttacatggcatcactactatttgaatggacggactgctaccagggaagattttctaaaccctgatgtattggacatttttatttcatctgtgcaaaaagttttccaggtggttgagagcaccaggcctggcaagaaggtctggttaggagaaacaagctctgcatatggaggcggagcgcccttgctatccgacacctttgcagctggctttatgtggctggataaattgggcctgtcagcccgaatgggaatagaagtggtgatgaggcaagtattctttggagcaggaaactaccatttagtggatgaaaacttcgatcctttacctgattattggctatctcttctgttcaagaaattggtgggcaccaaggtgttaatggcaagcgtgcaaggttcaaagagaaggaagcttcgagtataccttcattgcacaaacactgacaatccaaggtataaagaaggagatttaactctgtatgccataaacctccataacgtcaccaagtacttgcggttaccctatcctttttctaacaagcaagtggataaataccttctaagacctttgggacctcatggattactttccaaatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatctgaaaataaaatatactagtcctgacactg

SEQ ID NO: 10 - protein, 543 aa, Homo sapiens
MLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDYIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 11 - DNA, 1721 bp, Homo sapiens; identical to SEQ ID NO: 9
  CDS (63)..(1691)

SEQ ID NO: 12 - DNA, 824 bp, Mus musculus
ctggcaagaaggtctggttgggagagacgagctcagcttacggtggcggtgcacccttgctgtccaacacctttgcagctggctttatgtggctggataaattgggcctgtcagcccagatgggcatagaagtcgtgatgaggcaggtgttcttcggagcaggcaactaccacttagtggatgaaaactttgagcctttacctgattactggctctctcttctgttcaagaaactggtaggtcccagggtgttactgtcaagagtgaaaggcccagacaggagcaaactccgagtgtatctccactgcactaacgtctatcacccacgatatcaggaaggagatctaactctgtatgtcctgaacctccataatgtcaccaagcacttgaaggtaccgcctccgttgttcaggaaaccagtggatacgtaccttctgaagccttcggggccggatggattactttccaaatctgtccaactgaacggtcaaattctgaagatggtggatgagcagaccctgccagctttgacagaaaaacctctccccgcaggaagtgcactaagcctgcctgccttttcctatggtttttttgtcataagaaatgccaaaatcgctgcttgtatatgaaaataaaaggcatacggtacccctgagacaaaagccgaggggggtgttattcataaaacaaaaccctagtttaggaggccacctccttgccgagttccagagcttcgggagggtggggtacacttcagtattacattcagtgtggtgttctctctaagaagaatactgcaggtggtgacagttaatagcactgtg

SEQ ID NO: 13 - DNA, 1899 bp, Homo sapiens
gggaaagcgagcaaggaagtaggagagagccgggcaggcggggcggggttggattgggagcagtgggagggatgcagaagaggagtgggagggatggagggcgcagtgggaggggtgaggaggcgtaacggggcggaggaaaggagaaaagggcgctggggctcggcgggaggaagtgctagagctctcgactctccgctgcgcggcagctggcggggggagcagccaggtgagcccaagatgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctgggttctccaaagcttcgtaccttggccagaggcttgtctcctgcgtacctgaggtttggtggcaccaagacagacttcctaattttcgatcccaagaaggaatcaacctttgaagagagaagttactggcaatctcaagtcaaccaggatatttgcaaatatggatccatccctcctgatgtggaggagaagttacggttggaatggccctaccaggagcaattgctactccgagaacactaccagaaaaagttcaagaacagcacctactcaagaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatgaacctaacagtttccttaagaaggctgatattttcatcaatgggtcgcagttaggagaagattatattcaattgcataaacttctaagaaagtccaccttcaaaaatgcaaaactctatggtcctgatgttggtcagcctcgaagaaagacggctaagatgctgaagagcttcctgaaggctggtggagaagtgattgattcagttacatggcatcactactatttgaatggacggactgctaccagggaagattttctaaaccctgatgtattggacatttttatttcatctgtgcaaaaagttttccaggtggttgagagcaccaggcctggcaagaaggtctggttaggagaaacaagctctgcatatggaggcggagcgcccttgctatccgacacctttgcagctggctttatgtggctggataaattgggcctgtcagcccgaatgggaatagaagtggtgatgaggcaagtattctttggagcaggaaactaccatttagtggatgaaaacttcgatcctttacctgattattggctatctcttctgttcaagaaattggtgggcaccaaggtgttaatggcaagcgtgcaaggttcaaagagaaggaagcttcgagtataccttcattgcacaaacactgacaatccaaggtataaagaaggagatttaactctgtatgccataaacctccataacgtcaccaagtacttgcggttaccctatcctttttctaacaagcaagtggataaataccttctaagacctttgggacctcatggattactttccaaatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatctgaaaataaaatatactagtcctgacactg

SEQ ID NO: 14 - protein, 592 aa, Homo sapiens
MEGAVGGVRRRNGAEERRKGRWGSAGGSARALDSPLRGSWRGEQPGEPKMLLRSKPALPPPLMLLLLGPLGPLSPGALPRPAQAQDVVDLDFFTQEPLHLVSPSFLSVTIDANLATDPRFLILLGSPKLRTLARGLSPAYLRFGGTKTDFLIFDPKKESTFEERSYWQSQVNQDICKYGSIPPDVEEKLRLEWPYQEQLLLREHYQKKFKNSTYSRSSVDVLYTFANCSGLDLIFGLNALLRTADLQWNSSNAQLLLDYCSSKGYNISWELGNEPNSFLKKADIFINGSQLGEDYIQLHKLLRKSTFKNAKLYGPDVGQPRRKTAKMLKSFLKAGGEVIDSVTWHHYYLNGRTATREDFLNPDVLDIFISSVQKVFQVVESTRPGKKVWLGETSSAYGGGAPLLSDTFAAGFMWLDKLGLSARMGIEVVMRQVFFGAGNYHLVDENFDPLPDYWLSLLFKKLVGTKVLMASVQGSKRRKLRVYLHCTNTDNPRYKEGDLTLYAINLHNVTKYLRLPYPFSNKQVDKYLLRPLGPHGLLSKSVQLNGLTLKMVDDQTLPPLMEKPLRPGSSLGLPAFSYSFFVIRNAKVAACI

SEQ ID NO: 15 - DNA, 1899 bp, Homo sapiens; identical to SEQ ID NO: 13
  CDS (94)..(1869)

SEQ ID NO: 16 - DNA, 594 bp, Homo sapiens
attactatagggcacgcgtggtcgacggcccgggctggtattgtcttaatgagaagttgataaagaattttgggtggttgatctctttccagctgcagtttagcgtatgctgaggccagattttttcaggcaaaagtaaaatacctgagaaactgcctggccagaggacaatcagattttggctggctcaagtgacaagcaagtgtttataagctagatgggagaggaagggatgaatactccattggaggctttactcgagggtcagagggatacccggcgccatcagaatgggatctgggagtcggaaacgctgggttcccacgagagcgcgcagaacacgtgcgtcaggaagcctggtccgggatgcccagcgctgctccccgggcgctcctccccgggcgctcctccccaggcctcccgggcgcttggatcccggccatctccgcacccttcaagtgggtgtgggtgatttcgtaagtgaacgtgaccgccaccggggggaaagcgagcaaggaagtaggagagagccgggcaggcggggcggggttggattgggagcagtgggagggatgcagaagaggagtgggaggg

SEQ ID NO: 17 - DNA, 21 bp, Artificial sequence
  Single strand DNA oligonucleotide
ccccaggagcagcagcatcag

SEQ ID NO: 18 - DNA, 21 bp, Artificial sequence
  Single strand DNA oligonucleotide
aggcttcgagcgcagcagcat

SEQ ID NO: 19 - DNA, 22 bp, Artificial sequence
  Single strand DNA oligonucleotide
gtaatacgactcactatagggc

SEQ ID NO: 20 - DNA, 19 bp, Artificial sequence
  Single strand DNA oligonucleotide
actatagggcacgcgtggt

SEQ ID NO: 21 - DNA, 21 bp, Artificial sequence
  Single strand DNA oligonucleotide
cttgggctcacctggctgctc

SEQ ID NO: 22 - DNA, 23 bp, Artificial sequence
  Single strand DNA oligonucleotide
agctctgtagatgtgctatacac

SEQ ID NO: 23 - DNA, 22 bp, Artificial sequence
  Single strand DNA oligonucleotide
gcatcttagccgtctttcttcg

SEQ ID NO: 24 - DNA, 23 bp, Artificial sequence
  Single strand DNA oligonucleotide
gagcagccaggtgagcccaagat

SEQ ID NO: 25 - DNA, 23 bp, Artificial sequence
  Single strand DNA oligonucleotide
ttcgatcccaagaaggaatcaac

SEQ ID NO: 26 - DNA, 23 bp, Artificial sequence
  Single strand DNA oligonucleotide
agctctgtagatgtgctatacac

SEQ ID NO: 27 - DNA, 24 bp, Artificial sequence
  Single strand DNA oligonucleotide
tcagatgcaagcagcaactttggc

SEQ ID NO: 28 - DNA, 22 bp, Artificial sequence
  Single strand DNA oligonucleotide
gcatcttagccgtctttcttcg

SEQ ID NO: 29 - DNA, 24 bp, Artificial sequence
  Single strand DNA oligonucleotide
gtagtgatgccatgtaactgaatc

SEQ ID NO: 30 - DNA, 22 bp, Artificial sequence
  Single strand DNA oligonucleotide
aggcaccctagagatgttccag

SEQ ID NO: 31 - DNA, 24 bp, Artificial sequence
  Single strand DNA oligonucleotide
gaagatttctgtttccatgacgtg

SEQ ID NO: 32 - DNA, 25 bp, Artificial sequence
  Single strand DNA oligonucleotide
ccacactgaatgtaatactgaagtg

SEQ ID NO: 33 - DNA, 22 bp, Artificial sequence
  Single strand DNA oligonucleotide
cgaagctctggaactcggcaag

SEQ ID NO: 34 - DNA, 22 bp, Artificial sequence
  Single strand DNA oligonucleotide
gccagctgcaaaggtgttggac

SEQ ID NO: 35 - DNA, 23 bp, Artificial sequence
  Single strand DNA oligonucleotide
aacacctgcctcatcacgacttc

SEQ ID NO: 36 - DNA, 22 bp, Artificial sequence
  Single strand DNA oligonucleotide
gccaggctggcgtcgatggtga

SEQ ID NO: 37 - DNA, 22 bp, Artificial sequence
  Single strand DNA oligonucleotide
gtcgatggtgatggacaggaac

SEQ ID NO: 38 - DNA, 22 bp, Artificial sequence
  Single strand DNA oligonucleotide
gtaatacgactcactatagggc

SEQ ID NO: 39 - DNA, 19 bp, Artificial sequence
  Single strand DNA oligonucleotide
actatagggcacgcgtggt

SEQ ID NO: 40 - DNA, 27 bp, Artificial sequence
  Single strand DNA oligonucleotide
ccatcctaatacgactcactatagggc

SEQ ID NO: 41 - DNA, 23 bp, Artificial sequence
  Single strand DNA oligonucleotide
actcactatagggctcgagcggc

SEQ ID NO: 42 - DNA, 44848 bp, Homo sapiens
ggatcttggctcactgcaatctctgcctcccatgcaattcttatgcatcagcctcctgagtagcttggattataggtctgcgccaccactcctggctacaccatgttgcccaggctggtcttgaactcttgggctctagtgatccacccgccttggcctcccaaagtgctgggattacaggtgtgagccatcacacccggccccccgtttccatattagtaactcacatgtagaccacaaggatgcactatttagaaaacttgcaatggtccacttttcaaatcacccaaacatgttaaagaaattggtatgactgggcatggcacagtggctcatgcctgcaatcctagcattttgtgaggctgagacgggcagatcacgaggtcaggagattgagaccatcctgacagacatggtgaaatcccatctctactaaaaatacaaaacaattagccgggggtgatggcaggcccctgtagtcccagctactcgggaggctgaggcaggagaatggcgtgaatccaggaggcagagcttgcagtgagccgagatggtgccactgcactccagcctgggcgacagagcgagactccgtctcaaaaaaaaaaaaaaagaaagaaattggtatgactgttgactcacaacaggagtcaggggcatggggtggggtgtaagattaatgtcatgacaaatgtggaaaagaaacttctgtttttccaactccacgtctgctaccatattattacactcttctggtagtgtggtgtttatgtgtgaattttttttcatatgtatacagtaattgtaggatatgaacctgattctagttgcaaaactcactatgagcttagcttttaagttgcttaagaataggtagatctatgcaaataatgataattattattattattttaagagagggtctcactttgtcacccaggctggagtgcagtggtgtgattaagggtcactgcaacctccacctcccaggctcaaataaacctcccacctcagcctccccagtagctggaaccacaggcacgggccaccacgcctggctaattttttgtattttttgtagagatggggtttcatcatgttgcccaggctgttcttgaattcctcggctcaagcaatcctcccaccttggcctcccaaaatgctggcatcacaggcatgatggcatcactggcatcacataccatgcctggcctgatttatgcaaattagatatgcatttcaaaataatctatttttatttgttgccttattggtggtacaatctcaagtggaaaaatctaagggttttggtgttatttgcttactcaaccaatatttattagactcttactaagcaccaacatgatcacatgcctgagctatggctagcatagcgtgtgagacaaacttaatctctgttttggtggagcatataatctagtagatgaagccaatgttgagcaacatcacaatactaacaaattgaggatgctacgagagtgtctaacaaattgaggatgctacgagagtgtctaacaaattgaggatgctatgagagtgtgtcatggagagctgcctggagattgagagaaagcttccttgagggaagttacatttcagctgaaacacactgccatctgctcgaggttttgtaactgcattcacatcccgattctgacacttcacatcccgattctgacacttcacccagttactgtctcagagcttgggtccgcatgtgtaaaacaaggacagtatgcacttggcagggttgtgagaagggaagagaacacaagtaaagcacctgtatcaggcatacagtaggcactaagcgtgcgatgcttgctatgattatacatcagtgtaagcatcaaggaaaagctgaagaaaagtctgaccaacagcgaaagataaatgcgcagaggagaaatttggcaaaggctccaaattcaggggcagtccgtactctacactttgtatgggggcttcaggtcctgagttccagacattggagcaactaaccctttaagattgctaaatattgtcttaatgagaagttgataaagaattttgggtggttgatctctttccagctgcagtttagcgtatgctgaggccagattttttcaagcaaaagtaaaatacctgagaaactgcctggccagaggacaatcagattttggctggctcaagtgacaagcaagtgtttataagctagatgggagaggaagggatgaatactccattggaggttttactcgagggtcagagggatacccggcgccatcagaatgggatctgggagtcggaaacgctgggttcccacgagagcgcgcagaacacgtgcgtcaggaagcctggtccgggatgcccagcgctgctccccgggcgctcctccccgggcgctcctccccaggcctcccgggcgcttggatcccggccatctccgcacccttcaagtgggtgtgggtgatttcgtaagtgaacgtgaccgccaccgaggggaaagcgagcaaggaagtaggagagagccgggcaggcggggcggggttggattgggagcagtgggagggatgcagaagaggagtgggagggatggagggcgcagtgggaggggtgaggaggcgtaacggggcggaggaaaggagaaaagggcgctggggctcggcgggaggaagtgctagagctctcgactctccgctgcgcggcagctggcggggggagcagccaggtgagcccaagatgctgctgcgctcgaagcctgcgctgccgccgccgctgatgctgctgctcctggggccgctgggtcccctctcccctggcgccctgccccgacctgcgcaagcacaggacgtcgtggacctggacttcttcacccaggagccgctgcacctggtgagcccctcgttcctgtccgtcaccattgacgccaacctggccacggacccgcggttcctcatcctcctggggtaagcgccagcctcctggtcctgtcccctttcctgtcctcctgacacctatgtctgccccgccagcggctctccttcttttgcgcggaaacaacttcacaccggaacctccccgcctgtctctccccaccccacttcccgcctctcattctccctctccctcccttactctcagaccccaaaccgctttttggggggtatcatttaaaaaatagatttaggggttacaagtgcagttctgttccatgggtatattgcattgtggtggcatctgggctcttagtgtaactgtcacccgaatgttgtacattgtatctaataggtaatttctcatccctcatccctctcccaccctcccaccttttggagtctccagtgtctactattccactaagtccatgtgtacacattgtttagcgcccactctaaatgagcctttttgtttcattcattctgtaagtgttgaataggcaccacctaaggtcaggtataagtggaaatttgaaaaagaaactgcccacttgccccagtacttccctagccaagaggagggaaaccaggcaggtgcacctgaaggcctgtgagtgcttgatttgctgtgcagtgtaggacaagtaagattgtgcatagccttctgtatttaagactgtgttaggaagatttctctttcttttcttttctttttcttttttcttttcttttttttttttaggcagatgaaaagggcgtcacagaacaggaataaaaatctaaatattcaataaatgagacctaggagactactgcagtgacttacaaagtcctaataaaaagatgtctctccaaaatggggctgcaaaatgtggtgctgccttatcagctctaagttttttccttacctgagaaagaaggaacctgatgcaggttcagggctcctgccccatgaatgcaggctgactccaagatggggagctacagggacaatcccaggtcttctaggcctcttatttaggccctgggagcctccagagatggccacatcttgaccagcccagatagagggaaagatcaccattatctcacctctgtgtcaaatacctagatgctgtcctccctgagcccacactatagttgccagcgctaatttaatgggtagtgtactggttaagagatggacagaccatcctggcttgactctcagctctggcaaagatgagtgacttggtttttccatatctcttggccacaccaaccttgatttcttcagctgtagaatggaatttctcaagcttgcctcaaggattattgcccgaggatttgatgatatggtaagagcttctcagtgtttgacccatagtaagtgtttgacgtttcaaacgaattgtttctttctaggacatggtgagcatttggtagccattcaccggttttctgtttctttggatcatagttaacctctccttttccttctggcactacaattttctggtggggaagaatccttactttctgcccttccccttaaggataggaagctgatactaggcagcaactagttgggggataggaagattgttccagagaaatgctgaaccatagggctccagatcacaggaccccagtcttagcttgctggggtgtggggtgggggggggcggttactgaacatgggtatgaagtagatgtccatttactgaaatgtgaggacctgaggcctcttctattgctgtagccagcatattccccaacctctccccaagaaaggacagatgggggttcccccctggagtaacaggtccaaaagaaaaaacatacagtgggacttccaggatctgggcctgatcacccagcagtcaagctccccgcaattgactaacacccccctaacacgtagaaattccaatctgcaatttagtgaggatgatacctttattcttcttaaatacatctcttcatttcccagagcacccttttttcccctcctctgcacctttttgttaaagactggagtataatgaaataccaagagagcataacatgtgatacataaaactttttttctggtttacaaaacagttcattcttgtccatacgtgcttctctccaaggctggctgctgtctgttccagcccgcttcgcttggagaggccatctgccatacctgctccccagacgcatcgacaagcacacccagagtgttatctgctaagacctaaaagagggaggaaccccctctcctcatctaagacctagcttctaaattagagtgtgagggtccatctccccaggaggggcacagggcccaaacagcccagccatctcagaagacaacactaagctttgtaggggtccacagtagaggagagtaagacgcctgttgtttaatttattacagttcctcaaaagtgaagatgtgtgggcgggatggcaagagctgagcagacgaaagctgaaggaataaggaaagagaggaggacacaaacagctgacacttcctcagttcttgtcatttgcctggccctgttctaagcaccttctaggtattaatccatttagtcttggctacaacactgtgagtaactagttttgtcacccccattttaaaaatgaagaaagtgaggctcagggaggttaagtaacttggccacagtttgaaactagactctgatcacatgagataatagtgcccataaaaagggaaagcagattatattttttaaaggaaagagagtaggatatggtagaaaaagattgtttggaaaggaattgagagattgatataatgaaaagaagcattcacatgagagtaacagtatcagggcccaaaccttcatctaaggtacttcaaagaggcctaagcaaacttagtcactggcgtggttctagtctccatgatggcaaatacattgtgtacagcccaactccacacaaaacttaaataccaatgatagagcaatctaaaatttgaaagaaaaaatctttcaatttgtcgtcttcccagagggacttaatcaagaaaccaatcaaaatacttcctaagcctaactgtgtgcagaactccaaagagagcccagccctaaatcaacactgtccaatggaaatataatataatgtgggcctcatatgcaaggtcatatgtaattttaaattttctagtagccatattaaaaaggtaaaaagaaacaagtgaaattaattttaataattttatttagttcaatagatccaaaatgttttctcagcatgtaatcaatataaaaatattaatgaggtatttattattccttttctcaaaccaagtctattctataatctggcgtgtattatttacagcacttctcagactatatttctttctttcttttttttttccgagacaattttgctcttgtcacccaagctagagtacaatggcgttacctcggctcactgcaacctccgcctcccgggttcaagttattctcctgcctcagtctcccaagtagctgggactagaggcatgcaccaccacgcctggctaattgtgtatttttagtagagacagggtttcaccatgttggccaggctaatctcaaactcctgagctcaggtgatatgcccacctcggcctcccaaagtgttgggattacaggcgtgagccactgcacccggcctcagattaactatatttcaagcgttcagtagccacatgtagctagtgctatggtagtggacagtacagatctgcatttcaattaagacacgtatacaagcatagttcactaatgcacggtaaaaaaaagtatagtgctgagtcggtggtagaaatcctaaatactgcagagcaaaagtggtacgaacagcaatctcagtgataatgcaaccatgcttgcttttcattgcaatttgcttattttccttcagcaaagttcatccatttttgccaattcaataaatatttactgataaaaactttcaatattagattcttgcatcttcatagacagagttgcttttcacatttagaaaattacttatcaatgttaaacacacgttttgataaccagtgttggaaagaggtgcagactccccatgtgcctattgatggcagaaatattcacagccaaagggaaacaaagggctggggacaatcacacacctcatgtctcctaactcctgggaagtgctgtccctctgattgagctcttattattgccttccccactaaccctgtccactgtgccctggagccctttgcagggttacctgctctgtcctcctcacagaatatctcctctacctccttgtccaagctacaacttggctattctctgatgacactgtcttccctgtagcccttttgagtaatggctgcatattctcccatagtccagttcttttcctgttctccagtctggcttctggatgacagcccactagtttgaactccatactgctatagttcaagtcccttttgacttgttaccttgggcaaattacctccttttgttcaggttccttgtttgtaaaatgacgataataatgccatttgcttcagtgggttattttgaaattgagtgaaagaaggcgggtagcttccctacacgctcagtgtagactagcctgatgtgcattacgggtgatgccatgactcagtgtgttttcctcatctccacatctggctctcatccagtgctcctgcttacggcactctgtccccctcttacttactcccccttattaactgaagactggcactgatctcacagtttcctctccacttcctagtctcaccatcatcctagatgacttcaagtcacctagataaactgtctcagtttcttcactcacatttttttataacagataatgttacactcaagttgtaacagaaccagcttatccagctcatgaaatgtatgcatttcatctcaactctgtattcagtgacatcctgtgggtatctggaaatcagccatggtgagaatatttaccatggaaattggcaaatactaaaaagcagagcacctttttttctgagagccagaccatagctcttctactccatagcacccatcataacaatttttaaatacctccactgaacagcttcttcctctctctacttcttccatatctgatttgagcttcttaatttatcatgtgaaccactcttgtaataataaccccaaatccctgttccattgttcttcctgctaaaatactaaacctggtttagtccaaccatattttctctctttggaatctacagggtggcccaaaaacctggaaatggaaaaatattacttattaattttaatgtatattaataagccattttaatgcttcatttccagtctcagtggccaccctgtatagctgggctattgagctcttgcgggaggagggagtggacagtctcccagccacacagactgatgttgcaccaaacattttttagcttccagacttccctggcccttagtgttacccttaactctccatttctctgcctttcacattctctactttttaaaaatctctgactccaccttcaccttatcattcttagcacatgaccatacttctgcttcccaaagaaaatgagcaattacttccttttccttttcctcctgtcatcaaatctgcagacatgtcatgcctaagtccagctttcctcctttctctgatctcagtctgcttcttccatttctgccctgaatcccgtcccctccccaacccccaaggacttcgctctatcagtcacctcttccctctcctgtatcttcaactcctcccattttactggcttcttcctcaagcctttccccaagcctttcccatctcaattacctcctcgcacatgcctctgcagaaaccaccccgtttcttccctcccctcggcagcctgttcttcctgttctgccctcatgatggcaccatcattgtgtcactaaaatcaatctctccgacatcatcaatggccttcctttgttgggaaacctaataaacactttatcttatttggtctttgttatgggttgaatgaggttaccccgaaatccatattagaagtcctaacccccagtacctcagaatgtgactttatttgggaatagggtcattgcagacgttattagttaggatgaggtcatactggaatgtgatgggctgcttatctaatatgactgatgtccttataacaaggagaaatttggagacagacacgcacatagggagaataccatgtgatgacaggagttatggagttggagtcaaaaagctatgggaacttaggagaaagacctggaacaaatcctttcctgcgcctagagagggagtatggccctgccactaccttgaattcaacgtttcggcttttcaaaactgtaagacaatacatttctgttgttcaaaccaattagtttgcagtactctgcgactgcagccctaacaaactaatacagtctcttggaggcatttggcaaggttgacaatggaagcactttcttacccctttaggtctgtcgcctttcttgttggggggtgttttctaacaattcctctccatctctctctctctagtttgtcttaaacattggtgttcttcagacttctgacctaggccttcttttcacttcacatattcccctgggtggtctcacccacttccagaaattacttaaattactgctcatgcagtactgtgctggaaactgtttaacaactggctctctgggaagaggggagactggttgatggtttttgctgatttctgtggtgtaaatactccctccatggccaattccaaactgccaacagtttaacaactggctcacaaattttctccaaatttaacatttggctttcacaggccaacaacgtggtacagccaactccagcacacctctgcttttgtgtcagagagaagtaacttatttttgtacaaaaggtaaaataaaaacacctgcaggccccctttttttccttaacaaactgctctagaaatagaatagctgaagcttcttttatgcattcatctgttatttccatgtcactgtggtggtgggattatttttcctttatttttcttgtatatggttgaaatactgtacctttgatcagttttagttttatggcatgttttgcacccatattaaatctagtttttgtcagagggcgtcaatattattttctcaaaacaagaaaatatttcattgcaaaggagacaaacaaaaaggtccttaataccaaaactttgaaatgtgatttcttgtacttggcagtgtccaagtggtaaacccaaacagtattgggttttcattttgttcaggaaagtctttgtctggcagcgacttacccttacatcaggcgggccttgctcattcattcacttaagtatttattaaacaccagcggtgtgccaagtacttatctaggtatcgggtagattctgataagtcagtcaggtccctgctctcagggagcttgcagcagagatgggggctgcaatagagagtaagccaaggaaatgaaaaaggaagttgatttcagagagtgatgaatgctatgaagaaaatgaaggcagcgcagtgtgatggagagtgacccaaggtggtacagtttgtacctctaaggaccagactgtgacccaggtcactcacagatgcccgtcatgtgatgccacagcaacttttccaggtgctcgtttcctcccacttcccagtctcttgcccagccgcgactgcttacaaatacagctagaggaatctaaatgaggttcctctatcatcaaacccaatcaaaatgccaaggaacagaatcagtgcctggctgaaggcagtggaacagggccagcctggagtggttctctctgaggaagttcctcatcttggttttagggccataccttgtgacctgtgagctaggggttgccagtccctgacatttctactgaggactcgcctgtctatattcccggcctgtatgtgtctcctgagttccagacacacagggcgaagcgcctgatggatggaagtatgttttttggtgttccattggtatctcaaattctacaaaacttagtgccccttctcctccctgttcctccccatcttcagtctatcacctgttcctcatccagcaaatgatattaccatcttccaaggagcttcccaggagtaatccttgactcctcctcaacatccaattaataatcaaatctaggccaggtacaatagctcacgcctataatcccagcactttgggaggctgaggcaggtggatcatttgaggccaggagttcaagaccagcctggccaacaaggtgaaacctgtctcatttaaaaaaagttattttaaaaactcaaatctattatttctacctctaagtgtgtcttgaatttatccatctctctccatctctgagctgttaccttacctcagtccatcacgttttgtctacgttaacatgaccagagtcttgttcttagtctggtgaggtcactccagctgcttcagatccttccatggctcaccgttgccctcatataaagttggcactcctggacatgtggcttacggggccctccgtgatgtggccctatttgcttctccattctgttctctcccagcctctctgcccccatctctaggcaccaaccacacccttctgctcgtcaatggtgccagcttctcttctatctctggtctttggacagacttttcccttcacctggaatgctttcttcaatcctaccccactctctttaatctagataaggtttattctttttgaatgtctagcagtgaaaccatttcccctgaaaaaccttctctaaccaaccccctaccctcagcccaaggtctagattaggagtccctctgaatgtttccatagcatttttaaagaattgcctatttacttgttcgtatctatcactaaactacaaattgtatgagaacagccactatctctgcctggttcaccattcatctccagcaactagcataatgcctggcagagtcagcctgcaacaaatatttgttgaataaattaacagatggctttatctccttaagtaaatcttgcttttttcacctattaaaacagacgcacaggccaggtgtggtggcccatgcctgtaatcccagcactttggcaggctgaggtgggcggatcacctgaggtcaggagttcaagaccagcctggccaacatggtgaaaccccatctctaataaaaatacaaaaattagctgggcatggtggtgggtgcgtatagtcccagctactagggaggctgaggcaagagaatcgcttgaacccaggaggcagaggtggcagtgagccgagatcatgccactgtactccagcctggatgacagagaccctgtctcaaaacacacacacacacacacacacacacacacacacacacacacacacacacaccaagttgtataatttaaaatataacgtgcttgttatggaacacttgtaaaatacaggaaagtaatgaaaaagtctaccatctagctcaccacataatgaccattgctatcatcctggcataattctctcctgtatataaatatatattcttttattgttaaaattacactatgagtactatttatttattttactgtggcaaaatgcgcaaaacataaaatcttgccattttaaggtatgcagtttggtgcattcaccacactcacattgttgtgcaaatatcaccactatctatctcagaacttcttcgtcttcccaaactgaaactctgtacccattaaacaatagtgcatcctctgttttcccctccctacaatttatttttatttgggtttgtaccaaactgaaaatagctgcttcttccttacttagttcagattagcatttccatttatttagccgtggttttgaggatgccatgacagatgccatccttcctagagctctttggggctgtcaggtatttcagtcagggtgaattcgggttgataacattttaaaatctcactttattctgaggttcctagtgtcagagcccaccgtatttttagggactcccaagttacaaacaaaaatatggtgaggaggaatcactgaagttttaacacaagagacttacattttgttcaatttctatcttttagtttatttcctaagcataaagaaatactttgaaaattttacatagcattatacatatttaattaagcatgagcacatcttaaaactttaaattttagatcagatctttaattcctaggatattaagaggtactggcaatttggccaggtgtggtggttcacgcctataatcccaacactttgggagggtgaagtgggcgaattgctagagcccaggaggtggaggctgcaatggcctgagatcacgccatcgtactccagcctggatgatgagaatgaaatcctgtctcaaaaaaaaaaaaaaaaaaaaaaagaagaagaagaagtattggcaatcagtgctccaggaataatttcctgacttgaaataaacctacatgtagacaaactaattaggccattccaagagttgctagcattggtttaatatgttttcagagcattccaggaagcagtgtggccagcattgcatgtttgatacttcagaaatgtatgacaggtgtttctcttacccaggtcttctgttttcttagttttgctcatgtaaatatttatgaacatcctcatctttttgagggaagggattatagatcattctaattccattttctagcatttggtaccattctaagcacatgataggcacccatttggagcatttttggcttgacagaatatgcatttagaattgttcaaattagaggtgtcagtgatgggaattagaatactatataattctaagtcatttgacttaaatacaaaagaatgattttccttggtggggaatggtgaagggaggcaggagttaagaagaggagaagagatcctaagtcatttataaacttctctggaaagacaggtgtgtgaagactttttaaaaagtcattcaccaaattgtgtgtgtgtgtgtgtgtgtgttttaaatagactttattttttagagcagttttaggttcacagcaaaattgaatgcaaggacagagatttcccataaaccccctgcccacacacatgcatagcctccctcattatcaacatccccaccagagaggtgtttgttctagttgatgaacctacactgacacatcattatcacccaaagtccatagttcacggcagggttcactgtcggtgtacattctatgggtttgagcaaatgtataatgacatgtatccaccattatagtaacatacagagtattttcagtgccctgcaaatcccctgttctccacctattcatccctccctctctgcatttccacccccagcccctggtaaccgctgatctttttactgtcccatagtttcggacgatctatttttcagacagacacagagctgtctttcccttagtttctattctatcatttctttctccccatccatcataaaaggctatgagttttttttaagtgttgaacaccatcctacttgtcaagttaaaacataagctcctggctgggtacagtggctcatgcctgtaatctcagcattttgggaggctgtggcagaagcatcacttgaagccagaagtttgagaccagcctgggcaacatagcaagaccccatccctccacacacaaacacacacacacacacacacacacacacacacacacacacacacacacaaaaacaagctcttgccagaattagagctacaaattgccctcaggttcctagaagatcagtccttcaattagattcagattgagatgcttcctcttttaaacaatgattccctttctatcatgcccaataagaaaacaaataaaaattaaacaatactgcctgtaatctcagctacccaggaggcagaagcagaactgcttcaacccggcaagcagaagttgcagtgaagtgagatcgcgccactgcactccagcctgggaaacagagcaagattctgtctcaaaaacaaaacaatgtgatttcctcctctaagtcctgcacagggaaatgttaagaaataggtccaccaggaaagaaggaagtaagaatgtttgactagattgtcttggaaaaaatagttatactttcttgcttgtcttcctaacagttctccaaagcttcgtaccttggccagaggcttgtctcctgcgtacctgaggtttggtggcaccaagacagacttcctaattttcgatcccaagaaggaatcaacctttgaagagagaagttactggcaatctcaagtcaaccagggtgaaaatttttaaagattcactctatattttaattaacgtcagtccgtcatgagaatgctttgagaaaactgttatttctcacacctaacaattaatgagattaacttcctctcccctcatctgacctgtggaggaatctgaacaagaggaggaggcagtgggcaggtttccttatcatgatgtttgtcatgttcagtgtgaggcctcacaaaaaaaaaaaaaaaaaaaaaaggcgtcctggatataactgagagctcattgtacagtaaatattaataaaacagtgattgtagctgaaggatagaactgcttggagggagcaagtgggtagaatcgcgtcaaactaaagagcatttctagccaaagacacaatgatagattgaaggatatttattctaaatatagaatatgggtgaacgagatctgtggacttctgggctccaacgttagattctgattttagcaagcttgtcaggggattctgatattgaaaggctgtggccttcacctgagaaacctgccctagggggccatgaaaatttgtcctgtctttcagaagtgctatcagacatcaaatggaagttaaatcgtatcttaacaattactaggatgggcgcagtgactcacacctgtaatcccaacactttgggaggctgaggcaggaggatcacttgagcccaggagttcgggaccagcctgggcaacatagagagacgttgtctctattttttaataatttaaagagaaaaaaatactgaaaatattgtatacaccactgaattataataatgtgtatataatgtatatattcattatgaggaatatttgattatttcatatattatatcttttccttctgtttattttatccagttatgaagtatttagaacaattcatcagtaattggggctaaattgacagaatagtaatcagagaaaatagaaaaagacagatgggttatctttgaataccaggttggagttgtttatgggtttgttttttgttttgggggcgtttttttagacagagtcccactctgttgcccaggctggagtgcagtggcacaagcatggcccactgcatccttgacctcttgggctcaagcaatcttcccaccttagcctcctgagtagctgggaccacaggtgcatgtcaccacacccagctaatttttttattttttgtagagacagtctttctatgttatccaggctgatctcaaactcctgcactcaagtgatccccctgccttggcgtcccaaagtattgggattataggcatagccaccacacccaacctagtttctatttagacttggccctttcccaccagtcatttgtgtccaaaagatctcataaatgtagacaggaaactgtcctttgctcatcagttttcttcatcctgtgtctagggggatggtcggtgggggaaactggggttatgcaagttcctctgaaacatcctctgtgagcccagggatggatgaggcaccagccgccagcgagtcagtgtgcagctttccagaaaggaagtcatcagccagtcagccggccctggcagccagcacccggcaaccctgctgtcttgtgataaagaaatggtctgcctgacaggatggtgtggatttttcttttttcttttttttttttttgagacagggtctggctctgtcgcccaggctggagtgcaatggcgggatcttggctcactgcagcctctgcctcccaggctcaaggcatcctcccacctcggtctcccgagtagctgggaccacaggcacacaccaccacgcccaactaagttttcgtatttttagtagaggcagggttttactatgttgtccaggctagtctcaaactcctgagctcaagctatccatctgccttggcctcccaaagagctggaattacaagcgtgagccactgtgcctgaccagggtggattttttcaagtgcacatgttgtggtcccagaagctctgatggtaccaaattccaagcgaaaaaaagtcaatggttcccacccatcctacctcccatgatggcaagaggaaatcaccacactgcagatacagtccatgtaaaacaaattgctatggattttgaaagtgaaccttaagagaactgcactatgttttcttcattagagttctctggtaatttccagctttttttttttttttttttagacagtgtctcgctttgtcgcccagtgtcacccaggctggagtgcagtgacgtgatctcggctcactgcaacctccgcctcgtgggttgaagtgattctcctgcctcagcctcctgagtagctgtattttagtagagacgaggtttcaccatttggccaggctggtctcgaactcctgacctcaagtgattcgcccatctcagcctcccaaagtgctgggattacaggtgtgagccactgcacccggccagtaatttcaagcttctgaggagccctttgaattgttaaataacttgtagctatgtccaacatatccatgttcagtgtatgttcgatatttcttaggaaacctgcccttggttgttttctttgtggtaattcatgagccggcaaatttgacatgtgttacagaatataccttttctctgctctcctacctcataaccagaacttaattatcctgctttagtcacataaatagctaactaaataaatatatgagatttcagtctgctcactgtgaaaatagaccttctaaatgatctcttccacttgcagatatttgcaaatatggatccatccctcctgatgtggaggagaagttacggttggaatggccctaccaggagcaattgctactccgagaacactaccagaaaaagttcaagaacagcacctactcaagtaagaaatgaaaggcaccctagagatgttccagccccaaagatatttgaataggttggactcgggcaccaatctagcaagtcctacggaagttgtataaagctgaaaatactgaagcatttcccaaatgggaaatcctaaactcaaaacttgctttttggtttttttgtttgtttgttttttcttcatctgacattgcttagtagtcacagaatgaaagataaatcaatcattcatgatctaacaatgaccttcagtgctctaaaaaactacggagtcaaggaaaacatgaatatattcctcatgtaaaattaaaatacagacatataaagggcaaaacatgaacatcattcataccttgaggtccgtccccctcccagaaataacccccagtatgccttggtttagagcattaagcaggagggccctgagtcactccagacagtcttgaccaccaagcagcattctctttttgtttcctctgtggcttttgcaaacacagggctagctcagctacccattagtatgttttcagtcactaaaacagtcttccagtcttcaaattaggatgacattgtcacatggggctttaaagcaagtgaaacaaggaaccccctttttttttttttttgagatggaatctcactcttgtcgcccagcctggagtgcaatggcgcaatcttggctcactgcaacctccacctcccaggttcaagagattctcctgccttagcctcctattcattatgaggaatatttgattattcagttcctgtagggtaaagatattacccccgatcatattattgattattgagtagctgagattacaggtgcctgccaccacgaccggctaattttttgtattttttagtagagacagggtttcaccatgttggccaggctccaggctcgtctcgaactcctgacctcaggtgatccacccacctcagcctcccaaagttctgggattacaggcgtgagccaccactcctggccacaatccttttttaactatgaaatatatttttatctgaagtttgatgtttatacccaactgagggatgatgttcccatatctcagttaaagaaataacctgctcagatacttcaagctcttcttttgacttttgaaaataaatgatcttgaagttactatactttgtttgggttagttaacattatttaaagtatattattttaattaattatctttgtaagattttactgtatactacctggagttcaatgtatcagatggatttcaaatttatgtacattttttatgtatatggtacagaaaaaaatgtgatccataagaaatcagaaaatagcgcatatgctaatagctaatgttgtcctctaaaaaacttatttttgcatttttaagagggggatatactctgacactttaataagtgtaattaattattgactggaatttggcatgaggcagggccatttcagatcccattaaaggaatgacacataccagagaaccacagaagtaaggccacatttgtaataaatcattatagctctgctaggagaagacccagttgtattaggtaattaatggatttgctcttaaaacacatgtcccggaagatataggtgagtcttggggggccgcattaaacattataccaatgtatcttacatttctaagaaagttttactactttacaggatctttctgttaccaaaatggaaggtttccaactccaggacttggctttcatagttcctacaccaggggaaatgccttcctttgctaactatgcaaccaggttagttagtgtaagtccagccaccctgttggcaatgctaaaaggtacaacaaacacagaattttatttgcatttgtaaacatttgatttctggctcgaaattttcagttttcatgggcacgtcatggaaacagaaatcttctgtgtttagtttgggcacctactcattgtagtgacaaatatttcagaagccaataggggattccacaaattgttctgaacctgtggctgagactggtaatggctgagtgacatggggacataccacaaaagaagaggtagcaaaaggctgctgagataaggacatgttcattgcttagctagtggcctgcacccttaaaacacatgtcccaggctgggtgctgtggctcacgcctgtaatcccagcactttgggaggctgaggcgggtggattacctgaggtcaggagttcgagaccaacctggccaacatagtgaaacctcatttctactaaaaatacaaaaattagccaggcatggtggcgggcgcctgtagtcccagctactcaggaggcaggcaggagaattacttgaatctgggaggcagaggttgtggtgagccgagattgcgccaccgcacgctagcctgggcgacaaagtgagactctgtctcaaaaaaacaaaaacaaaaaacaaacaaacaaaaaacaacaacaacaaaaaaacgggtatcccagaagatacaggtaagttttctaacacaggtcctcttgtatggtgcgttccacttaagtagaagatgacaaaaacatttgtcatgagaatatagactcacattttaaacctgtttgagcaggaaaaggaagcaatgttacagatgtaattctgggtgtgactgcagaaaggatgactcccttattaaagtagtcatcctgagtgagctaactctttgtacttcctcttctcctcctgttcccctcatcaccccattcttccgttgcctacacccaggcccacattggatgctgacatagacttacatggtacagtccaagggaaagatctgccatttttttcaatgtgtcatcttggttatcttcattccaaggatctctccactctttatacagtaagagatgagagtctggaaaggattgggaataagataatgaattgtaagttttaaattgttcttcgtattttggggaaggagtaggctaggtggtccttctgttttttttttgtttttttttttaaagtagatgtggccagacgtggtggctcacgcctgtaatcccagcactttgagaggctgaggcaggtggatcacttgatgtcaggagttcaagaccagcctggccaacacagtgaaaccccgtctttactaaaaatacaaaaactagccgggcttggtggcgtccacctgtagtcccagctactgcagaggtggaggcaggagaatcacttgaacccgggaggtggaggttgcagtgagccaagatcatgccattgtactccagcctgggcgacagaacaatactctgtctcaaaaaaaaagagaaaagaaaagaaaaaaagaatggatttgaactcagtcgtcaatagcctctattccaggagatgttacagttgattatgttatagggggtgtataatagaatttcgagctatgtaaattccaagtgcatttggaagaatgaagaaatggaggaagggtaaagtatgagtgcaagcattccaggttttttgaaaatgctataatctttgttcagggctagtacaaagtgctatttagctgtaagggttttttgtgatttacagacagttttcacatgtgtcatttcaaccttggttttatggcgaaggcatgtgatggtgcttgtcccaggactttagatccatatctgaggttcctgtcgggcaaagatattacccctgatcatattatagtctataagtgggagagttgtgcctggagctcaagtcttatgatttctgatccagggcacttcctacaacatgattttgcaatataaaagcctataatgtgtgactaaagcaggtcactcaccccttgtaacagactctagtaatggtactgccaccaaacggctgcgtgatattgggcaaagacttaccttatttgaatctcagtttcctcctagaaaaatgagggtggaggttaagcataggctgatgatcctaaagcctccatactgccctaaactgtggctctaagatccagtagaatgctgggtcacaggactctagggagcttttcaaacccaaatgtctgtcattccttgatggtaggcagcagtttatggaagtgggcgacacagcaaatatcaaaatacctaaagcagcttgcaagagttgtttctgcctagtggtctttatagttaatattaaatagttaattttttttttttttgagacagagtcttgctctgttacccaggctgcagtgcagtggcacaatctcggctcactgcaacctccacctcccgggtttgagcaattctgtctcagcctcccaagtagctgggactacaggtgcatgccactgcacccagctaatttttgtatttttagtagagacggggtttcaccatattgggcaggctggtctcgaactcttgacctcaggtgatccacctgcctcagcctcccaaagtgctgggattacaggcatgagccactgcacccagcttaaatagctaatatttaatattattctatagttattcaagtaattcaggccaaagacttagaaacaaaacaaaaagccacttttaaggagaaagggtgtaagtttgccagatagatagagatctttcttttttaactacaagagttcaggaatgaattactctttaacaaacgactatagatatacatgaaaattggaaggacttattatgcatatgataatcaatttaaagacaacacttaaaattatattgttgccactctcaaaaagtggtaatagaacagctaatggtttaaaaagcagagtacagaagttcccaaacttatggcaccttaatatcgcagaaaactttttaaagcatgcctaggccacaaaaaatacctgtattttgattattaaattgtaaggtctacacaacctaatagtaataggtccaatagtaatgctgtccaatagatgttgatgtttttttccttgcaaacttaaaagatcctacagtgcctctgtaaatagcactgcctggttagagttgaatttcagataaataatttttttcatgttaattatttttcttttctttacttttttttttgtttttttgtttttttgtttttttttttgagacagggtctcattctgttgcccaggctgctgtgcaatggcatgatcatggctcactgcagccttgacctccctgggctcaggtgatcctcccacctcagcctcccaagtagctagctgggactacaggtgcttaccatcatgcccggctaatttttgtgttttttgtagagatgtggttttgccatgttgcccaggctggtcttgaactcctgggctcaagtgatccgcccgcctcggcctcccaaagtgctaggatgacaggcatgagccactgcacctggcccctgggcgaagtatttcttaatggttacataggacatacactaaacattatttattgtctatatgaagttcaagtttaactaggtgccctgcacttttagttgctaaatcctgtagctgtacccatgcattcactggtgctccccagcttgccttgcacagagtttggaaaccatagtcctataactctaggccaattttttaatgtaaaatttgattcattttaaattaataaataataacaggaatttttttaaaaattgttttaaatataattaaaattatcaaaatattttttaactgaacttgtgactagagatatttagattatgaagagtggggtttatgctaactaatgacagtctggctatgcatgtggagcactgagctataaattgtggcttccccaattctcctgatgtcacttgaacaaaacctaagtgtcagaccagagcttctggtatcttccatgggatttcattcaacagctggagcaaatgaagtcagattgattttttttaatttgtccaattttgttgtctcaaaaacataattataatcatttattagaactagaatttcttcagtttaacaacagaaatagttattcattatgaaaagcgaatctggaggccttcattgtggtgccaatctaaccattaaattgtgacgtttttcttttaggaagctctgtagatgtgctatacacttttgcaaactgctcaggactggacttgatctttggcctaaatgcgttattaagaacagcagatttgcagtggaacagttctaatgctcagttgctcctggactactgctcttccaaggggtataacatttcttgggaactaggcaatggtgagtaccccagggaacaattcattaataaggagattccccactagcattatttcttttcttttctttttcttttctttttttttttttttgagacagagtctcgcactgctgcccaggctggagtgcagtggcgccacctcggctcacttgaagctctgcctcccaaaacgccattctcctgcctcagcctcccgagtagctgggactacaggcacccgccaccgcgcccggctaatttttttttttttttttttttttttttttttgcatttttagtagagacggggtttcaccgtgttagccaggatggtcttgatctcctgacctcgtgatctgccctcctcggcctcccaaagtgctgggattacaggcgtgagccaccaggcccggctagcattatttcttatgacactttttttttttttttgagacggagtctcgctctgtcgcccaggctggagtgcagtggcgccatctcggctcactgcaagctccacctcccaggttcacgccattctcctgcctcagcctcccgagtagctgggactacacgcacccgccaccacgcccggctaatttttttgtatttttagtagagacggggtttcaccgtgttagccaggatggtctctatatcctgaccccatgatctgcccgcctcggcctcccaaagtggtgggattacaggcgtgagccactgcgcccggccaacactctttttattattagcaaatatacttctgcctgggcacattcttgcaagtgctcaacaatgcaacttttggaagtgcatgtggcagaaactcctgctgtatttattccagaacctattattgctaatcccagtttatgttacatttgaagtgagaaccagttggagccagcaacgttcccagctccaaagttcccttgagattttcagaatcacttaaccctattatgcttggcaacctggactcagcaaaactgggaagtcagcagtttgttttattcatcccttcctttctcagtttctcaaatgtgtcagttaatctcagtaaccccattgcaaccttcattacctgcccaagcggtctagaacttgccagtatagaatcctacgtgggtcaagctcctgactgtctccttcttcactctttttttgcaaagaacttgtaaattttaactataagtattcatgattcgccacatttattcaaaacatagagtgctttttccacatatcagccaatggaaataaggattaaatgggaaatgaaatgtagtaataggataagcacaagtcttcttcctgctcaaacttttttttttttttttttcagacaagatcttgctctgttacccaggctggagtgcagtggcgtgttcatagctcaatgtaacctccaactcctgggctcatgcaatctctcacacctcagccccctgattagctaggactacactatgcctagccaattttttttcttttgtctggttgtgttgcccaggctgtctcgatctcctggcctcaagtaatcctcctgcctcggccttctaaagtgctgggattataggcatgagccactgtgcccggtctcaaacctttttttccaaagtaaatgaagttattagatatggaatatagtctagttcccagatatccatatccattggtttattaccctcattattaacttcaaattgtttaatagaccctcatatctcagttatacagttaaaatttttgttttgtttttctggagtatcttatttataactatgagttttactttacttatttattttattttttgagacagacgcttgctctgtcactcaggctggagtgcggttgcgtgatcatggctcactatggcctcgaccttctgggctcaagtgatcctctccctcagcctcccaagctgagactacaggcatgcaccaccacatctagctaatttttttttttccccatggaacaaggctttactatgttacccagagtggtctcaaactcctggcctcaggggatcctcctgtctcagcctaccaaaatgctgggattacaggcatgagccatagcgccagacctggttttacttttcttgactttgaattacaagtttttgtaatttggaaaatgttttgttgcttttaaatactgctgtatgtttgcttttaaatacaacatttctcgatatatattttgagaattgctgtctttcagaacctaacagtttccttaagaaggctgatattttcatcaatgggtcgcagttaggagaagattttattcaattgcataaacttctaagaaagtccaccttcaaaaatgcaaaactctatggtcctgatgttggtcagcctcgaagaaagacggctaagatgctgaagaggtaggaactagaggatgcagaatcactttacttttcttctttttccttttgagacagagtctcactctgtcagccagactggagtgcagtggtacaatcatggctcactgcaacttcgacctcccaggctcaagcaatcctcccatctcagtcccacaaatagctgggactacaggtgcacatcaccacacctggctactttaaaaaaatttttttgtagagatggggtctccctgtgttgcccaggctggtctcttgaattcctgtgctcaagccatccttccacctcagcctcccagagtgccaggattacaggcatgagccaccacacccagccaccacttttcttaaaaaaaaaaaaagattctctctggtagacaatcctcaatagtccacatgttattaaacaatctgctgcctgaatacatgatttaccaaaaaaaggaaattttgacgggttcagaatatcaagggatctgaggcaaatgtcacctatgataaaatttgctatcaaaattaggaagtttgtgtttacctgatcctaaagcagtaaccagcccatttctagggaataaaactctcatgcgtatattgtgcatatatatgtattatatgactgagtgataataaaattttttttctagcttcctgaaggctggtggagaagtgattgattcagttacatggcatcagtaagtatgtctcctattcttaatactaggaaagtaaggctagctttatttattacctagtattcaaaaagttagttcatttaactgccaattgactgcagttcaaataagaaacaaatagtgtctcaagtagcactgtactccaattttaatattaataaaaaaaattttaagttattttaaataatgtagtggtttctataaagatcactttatacagaagaacagtgccaattaacccatggaacatataagtagctaaaaccaattgcttgccaaagaaccagtaacccaggagtacatgtccttgccactgtgttttttcaagacagagtaactgatttctagttacttgcatagaatggactcctcctcataactcccttccatcttggtctttccctagtagaacttctacctttttttagtaacaggtgagtgggagaggtaagaaggagaataaggtcagcaattaacctaaaagcagaaagtaaaatttgttattttttttctgaatattttctgtgtaatttagctactatttgaatggacggactgctaccagggaagattttctaaaccctgatgtattggacatttttatttcatctgtgcaaaaagttttccaggtaatagtctttttaaactttttaatgtaaaaccagaatccttattttatagtctagctagttctaaattctataggtatgtatatttacatgtttttctaattttagagaacaagcactatgacttatccactgttagttttccccttagcattgggtcttaccccatgtacgtgattagaaatttgaaatatttccaatagcctttagtagaattaactcacatagatgataagaatgggttggttcacttcatgttccttccacagcctactatttcaataaaagaaagtttcccaagacctaaatgactatgaacatattttataactatataggaggggtgggtctaggaatacaaagttttgaatgctgttaatcttcaacaccacagttgaaaccacaggtcagcttttttgcaattaccatggatacttttctgttctataggtggttgagagcaccaggcctggcaagaaggtctggttaggagaaacaagctctgcatatggaggcggagcgcccttgctatccgacacctttgcagctggctttatgtgagtgaagcagcgctggccttaggggtcagagtgcagctcttctccatccttctattctgctgaaatagctccccagccaaaaagcagatcaaagaccgtttcagtggctgagccccaaaattcatgccagattttgcaagaaaatgatttactaaagcttgagggacatctttaacaagtgttccaaattaatcactataaggatgaattgtttcagaaattttggcctttaattatggcccataaatatgtcaagtagtccttactctaaagaagtacactgtaaaagaatgcatatagccggatatggtagttccctgtaatcccaatactttgggaggccaaggtgggaggattgcttgagcccaggagtttgaggctgcagtgagttatgatggtgccactgcactctagactgggcaacagagtgagactgtctttttttttcccctctgtcacccagactggagggcagtggcacgatctcacctcactgcaacctctgcctcccggattgaagcgattctcctgcctcagcgtcctgagtagctgggactacaggagtatcaccgcactgggctaatttttgtatttttagtagagacggggttttgacatgttgcccaggctggtctgaaacccatgagctcaagtgatctgcctacctcagccttccaaaatgctgggattacggacatgagctaccacgcccggccacaccctgtctcttaaaaaaaaaaaaaatgcaagttagagcatattacagctttgtctctcaggaggatacttagtgtatgtagctataattcatagattcccaagaagtttagagcctaaagtatgaggtcccaccagaggggctatcattaaatttaaagatttgttaaatcatctcattgtccaacaccacaaacttgattgctttaaaatactggtttagttacatttagtaactctattagtgcttttaatctatactgctatatcctcacattgagattttttttcttttctcttccatcttcattcttttttctctcatcctcattcttataagcctagaatacatcacaaatcctttatgcccatggaagcaagaggaataaagaatggagatgtttgttttgccattaactaaagatctggggtgtcggggagaagggggatagagaaggagaagtgggaagaggtgtccataatagcttaggtgcaattctgcttattttacattttacccccgctgactgccactttttcttcagccctcacacattgtttgtgcagggacctcataggaccaggaattgtctatagaggtgggaatttgtctcaccctgaaagggatacctctagcatggtaatagtcttctaggatttgttatcatatggaaagatgtaaagggagggattctgctgctgctgctgctgctgcatgcagttgccatttcatttaaatgacttatttataattgatgacacttttctggcttcctgttaattcctccctcaaagatcaataaaccagaaccaggcatggtggcatgcacttgtggtcctgtaaccacccaacaggttcaccttgcctgctgtctagatagagccaattatcaagacaggggaattgcaaaggagaaagagtaatttatgcagagccagctgtgcaggagaccagagttttattattactcaaatcagtctccccgaacattcgaggatcagagcttttaaggataatttggccggtaggggcttaggaagtggagagtgctggttggtcaggttggagatggaatcacagggagtggaagtgaggttttcttgctgtcttctgttcctggatgggatggcagaactggttgggccagattaccggtctgggtggtctcaaatgatccacccagttcagggtctgcaagatatctcaagcactgatcttaggttttacaacagtgatgttatccccaggaacaatttggggaggttcagactcttggagccagaggctgcattatccctaaaccgtaatctctaatgttgtagctaatttgttagtcctgcaaaggtagacttgtccccaggcaagaagggggtcttttcagaaaagggctattatcatttttgtttcagagtcaaaccatgaactgaatttcttcccaaagttagttcagcctacacccaggaatgaagaaggacagcttaaaggttagaagcaagatggagtcaatgaggtctgatctctttcactgtcataatttcctcagttataatttttgcaaaggcggtttcagtcccagctacttgggaggctgagacaggaggattaatggagcccaggagtttgaggttgcagagagctatgatcacgccactgcactccagcctgggtgacagagtgagaccctgtctctaaataaataaataagtaaataaataaatacataaataaaatcaagatggtgtgcaattagaattgagcgattttgtttccaaacctcaagaaagcttggtcttgctctgtcccaggtggctggataaattgggcctgtcagcccgaatgggaatagaagtggtgatgaggcaagtattctttggagcaggaaactaccatttagtggatgaaaacttcgatcctttacctgtaagtgaccattattttcctaattctagtggagtagattaaagtcaactcaggacctctggtgttaacctcctatgaacagtcagtcctctcagtaactagccaaatcatgagatgatgaattagaaggagccttagatagcatccaatctaacatttttttgtgtgtttgaagagaagaaatcaagagctaggaataactttttaaaggtaagccatttgcagtatagtgtggattttgtttaaaaggggataatttgaaattttatgactcattatacaagacaaaataagttggattttcaaatgttttacaaagtaaatcaaagttataattgcctacagtacgcaaagcttcaaaacattttttatgttatgaaattgtaatttatttaaccttaaaatgagccagtaccatgtgtttgcttaaaaatctcatgctaagaatttactatgttgttaataatcttcaagatatttatgaataaagtcttatttctaatccttcctccaactgtatctggtgctaaatcaggaaatgtttcttcccaaaaagcctcgtggaagatctgtatgtctaaatatatgtcagggataatacagatgtagccctgcgaagcatgaccttgatttttatagtctaaaatgtcatttgcagatatctattttctaagaataattcctaaaagaattatttgaatgttgtaggaaagctaagaaattttgcaaagagcgtacgtgaaaatataagctaggcttttgtggtttgtggatagacttcccaacaaaattgctttttatctatagtgatccaagcttgtggaacatattagtcatctttttttagaaaattcttagaaaagtgatcttgcaaaaatggaatttatctttccccaagtatattctgtcatgtatagagttaaactaagcatagtaatttcaccagacaaacattcaaaatctactcctgacctttttatctcatccaaattttcccagggcccagacataaacctttgccttacgaactctttgtatatgcactaaatatgcttctccttcaaggttctcagtcagctagaaaaatgtgcaagagtaaatggtacccttctcacttgtagatccaagagaattagacttaaactcactctacatgtctgtgactttattttatttgcatgacagtcctgtgaggtggcaaggcaggtatcttggatccattttttagataaggaagttcaaattgagaagaggttgcatgatttacaggaagccatactgtagtcctatgttactcttaaaaatcccattcaaatcctgcttctgaggcctgcatactttctaccctaccagtcattgacccatgcttatgtctcctttgaaaacattgattccactcttgtctccagtgaaaaagtggaatttaagcagagaaacaaaagccatttgtcttgttaagtctactttccctctactttcaagaaggaaagttggggtatgtgttgaatggtgatttatttatttatttattattttaaaaattgatacaaggtcttactgtattgtgcaggctggtctcaaactcctgggctcaagtgatcatcccacctcagcctcccagtgttgggattacagcatgaaccattgtgcccaccaccgatccgcagttttttaagaaaaacttttactatagaaaattttaatcatatacaaaatacagaggaaagtatatgaacccactttaggagactagaatatgccaccccaaaatatgccactttggcataaggattatttcgagctaaaggcaactgggaagaaacacatagaagaaaagttctctgtccttctccatttgcctaaaagcaggacatgaatcttaaaagtccccctccttccctttctaccaggaaaaacaagagttaatcactgaagataacttcagacccttatcagtgtagagatggcactagaagaatctatattacatactcatttattttccttcccacaacttgccaccccagagactaaaaatccttttcctttgtcatgtctcttgtccaaaaatttgctctataagctggagttctaagccacctctttgagaattacttgttccctggtattttctgttaacatacatgtattaatatacatgttaacaagcttctgtttgtttttctcctgttttctgtcttgttacagaggtccatcccaactaagaactaaagagtaggaggaaaatataatttcctcctgcatactttgatcttgtttaatccgtaacccttcccacttttcacctcctacctattagattactttgaagcaaatttcagatatattactttatctataaatatttcagtatgtgctaggtgtggtggctcacacctgtaatcccaacactttgggaagctgaggcaggaggatcacttgagcccaggagttcaagaccagctacggcaacaaaaaatcaaaaacttatctgggcatggtggcacatgcctgtggtcccagctacatgagaggctgaggcaggaggatcgctttagcccaggaggttgaggctgcagtaagctgcattcacaccactgcactccagcctgggtgacagagtaagaccatgtctcaaaaaaatacatattttagtatgtatcctttttgtaaaaacacaatacttttatcatactttaaataataacaataattccttagtatcaccaaatattttgtcagtgtctcacattttccttattgtctaaaatattgttgatagttattcaaatcagaatccaaacaaggtccatatattacatttggttgacaagtctcttaagtttgttcatctttaagttcttcctccctctctttcatctcttgtaatttattaatgtgaaaaaacaggtaatttgttctatagtatttcctacattatagagtttgctacatttattccctatgatatcatttagcatgttcctctgtcccctgtgtttcctgtaaactggtagttatacctagaagcttgagtttattcaggtttttaattgtattttttttgcaagaattctttattatctgcttctggaagcacagaatgtctggttgtgtctggttttgatcttgacagctactgatgaccattgcctaatccattactttattggggtggggggaataaggttttaaaataaattttttttaaagatttttttaactgttattttgagacagtgtctcatttcgtttcccaggctggagtgcagtggcacaatcacggctcactgcagccttgacctcctgggatcaggtgatcttctcacctcagcctcctgggtacctggaactacaggtgcacaccaccacacctggctaattttttgtattttgtgtacagaaggggtttcatcatgtttcccagactggtcttgaactcctgggttcaagtgatctacccacttcagcttcccaaaatcctgggattacactttggccaccgtgcctggcctaaatgaaattatttgtctctaaacagacagaagttttactttaaaaatttgtctttgtgtgtacatgtgtttgtgtatgtgtgtgtgtctaaaagtttggctttgagctttgctttgaattcttggatgaacaataaccaagaatacttaaactctgatcattcttgacagatatcccctacaggctatggccttttgaattgtgtcctccagtgataaaaagcagcaagcacgatactgctctcagattcatggtggtcacatgtgaggtgaaaaaaaaaaaaaagatgaatcctatttaaatgcccccaggataacagtgatactctttgtaggataactatttgcttgccactggtttcattaaataaggacataagtaaagatctatttttgtctctttctccccaaccaccacaactaggattattggctatctcttctgttcaagaaattggtgggcaccaaggtgttaatggcaagcgtgcaaggttcaaagagaaggaagcttcgagtataccttcattgcacaaacactgacaagtaagtatgaaacacaccctttaccaatcatcaagttttagtgggtaagcctgtaactttactcaaacaccctgttgcatgtgtctatacattgcataagtataggcagttgcaatttagtaaagttttatacaacgattttattttattttatttttagaagaaaaatgctacttttgttgttgttgttttttgagacggggcctcgctcgtcacccaggctggagtgcagtggtgcaatctcagctcactgcaacctccgcctcccgggttcaagtgattcttgaagaggagaacaataataacaacaatattattttcaaaagttgtgaccgcagtttctggagttgagaagacatcgagatttttgtagcctcatactcttgctttaggtagcaaaaaatgttcctaaatctcaggaatattctctagataggtttcaatctatcattcctgataagatgatgctgaaatactaattctagccaaaaaagaccagctaccatttccgattgttggggactgggaactctggatagtgaggaccccagtaggaagtagcgaggggaatggtttgaatggataaattcataaaaaatgtcagtagatttaattttcttatacatttcagtctttttataaggctaggaaaagcccctgtttttatggtttataatttgaattcacatgaacccacaaaatttgccttttaccttcctatgtctgaaaatggatagtctggctggcctcttaacaacccagctggcagagctgtgaggatctcagtgtgctctagcccagacattggtagcatgaacggcaacatttttaattgtgttttcaaaataggagcacactagcggtctaaaacgatcataaaagaaggatactaagagggcccactgtcattatggatcctaatacttaggatgcattatggattgtcattatggatactaatacttaggatcacatttgtaattgagtttttaattgcttaaattagatacatatttctattaagttaacctctttgcttttagtccaaggtataaagaaggagatttaactctgtatgccataaacctccataatgtcaccaagtacttgcggttaccctatcctttttctaacaagcaagtggataaataccttctaagacctttgggacctcatggattactttccaagtaagtaattttccttgttcattccaaactttcaataaatttattggtgtttatcagaatagagagtttggacagggagcaaaagacaaagtcaactatatcaagttctaataattcttaatattcaggaaatttatgtatgaatacttactaatatgagtataactcatcctaagagtctaaagcaaaaggatgtgaacacaaactagcagttatcttagagaataagtttgcatttcaaaataacttgacatatcaagatccactcaacgcatttaaattatttactctaaaaagacataattcttggtaacacattcactaaagcaaaatatacctttatataattgctatcaaaggtatgtgggttggtataaaatatcataccatgtgagatcagtgtgattcctttacagcattaatttttattggttagagtaagaaaaagaatagctagagtatatttcttaagtagattctcatacactttggtttcaaaaaccaattattgactacatcttataaaagcctgtattcaatggagtgccaaaaaatgactatgagtcttaaagagttaggcatataaatattttaaggtttctgttcaatgtatgttggaaggagttcctttctcatgactattctcatattggagcataaaaagagtttacaggcttggcgcagtggctcatgcctgtaatcccaatactttgggaagctgaagcaggcagatcacttcagcccaggagtttgagaccagcctgggcaatatggcaaaactctctctacaaaatataccaaaattagccaggcgtggtggtgcatgcctgtagtcccagctacttgggaagctgaggtgggaggattgcttgagcccaggggggtcatggctgcagtgagctgtgatggtgcctctgtcacccagcctgggtgacagagtgagaccctgtctcaaaaaaataaataaataaaaattaagagtttacaaaattctcaccatctcctcccatctttgcaaatgccacataagtgatgtgttccaggactattagcctcggaacctgaggcagtacagtaagcacgctttctccaaagtcctgtcccccacagacaaacattatttacactgggtactgctcttttattttttcccctctatgctttattttactataactataatcatataacatgtaataggaaaaaggcagggtcgggggagagatccagaagtcttcccaagagcctttccaacatagcctctgtagacattttttctttcttctttttttttttttttttttttctgagacagagtctcactctgttgtccaggctagagtgcagtggcgtgatctaggctcactgcaacctccgcctcctgggttcaagcaattctcccacctcagcctccctagtagctgggattagaggcatgcatcaccacgcctggctaatttttgtatttttagtagagatgaggtttcaccatgtgggccaggctggtcttgaactcctgacctcaagtgatccacctgccttagcctcccaaagtgctaggattacacgagtgagccaccgtgccctgcccctattacattctgatcacacatttcatgttttataattggaaaactggtgaaattatagacaatgttttgttcccctaaattctctttgatgagtatatattacttacactcttctgtctttaaaattttgcaaaatagtatcctagataagtttatgagtgcacagtctgtacgcttactcatattaatgacctcggagagttaaacaacagtcacctttaaaaattattactatcattatcattatttttgaggcgggggtctcattctgtctcccaggctggagagtagtggtgcggtcacagctcactgcagccaccgctacctgggctcaagtgatccttcctcctcagccttctgagtagctgagaccacaggcttatgctaccacacctggctaattttttaactttttgtagagacgatgtctcattatgttgcccaggctggtctcaaactcctaagctcaagtgatcttcctcagcctcccaaagtgctgggattacaggcatgaaaaactgcacccagccctaaaaattattagggtcctgcatagtaagactttaataaatatttaaatgaacatctggtttttttaaaaaaaaaatagagacaaggtctcactatattgcccaagctggtctcgaactcctggactcacgcaatcctgctgccttagccgcccaaagtgctgggattacaggcatgacccacctcatctgggctgagtgaacatatttttaacataaaggccgtattttatatttatctcatacattttgcccagcatccccatttccgccgaatctgttgcttgctaattccttccagcttcatttcatctgaaatttgacaaacatcttctatttctttgtcgtcatgttattgacttcagaatataaaataaaacactatacccaaattaaaccccaccctcattgcccagcctgatgtgaaaataatcagcatacattaagcttacccttgatatatgtgtagcatcttttagataaatatacagctgattaagcaatatagcctgatggtataatatcttgcccatgtacctcatcttatctccagcaggattaattcacagtgatcagatttacctttaaactttgtagcaaaatatcctctccaaaagcatatctaaaacttttgtgtgtactcttgcaagtttcttaatttcatgcagaacaggctcttaccactgttagctggagatattttcaagacctatttttgtttgtggtttcctgatgatggtcatggcatttcccccttcactccatctaaaaattgaggtgatacaggcttttaaacaaaaccaactcatatagactgagtacaactgcaatgcaggcatgctaacctctgctacaatcatgggcgtgctattgatatgtcttaagttacagaacacagggctgagcgtctcattaggtcaaaatgtaaaccagtttttctgctcactgatgcttaatgaggacagggtgtgagagatttctttaaggaaaacaaatatataataatgctacatggaaaaatatctaacattagagaattaagtaaataaactaatatactcacaccatggaatcttgtgcagacattaaaattatgtagtggatggatgtttaatggtgtgagaaaaagttaggatgtgctggggtggggggaagaatcaagttttaagaaaatacagtatacccatacttaagtaaaaaaaaaaaaaaaggtatgtacagtcatgtgttgcttaatgatggggatacattccgagaaatgtgtcgataggtgatttcatccttgtgtgaacatcatagagtgaacttacacaaacctagatggtctagcctactatgtatctaggctatatgactagcctgttgctcctaggctacaaacctgtaaagcatgttactgtagcgaatatacaaatacttaacacaatggcaagctatcattgtgttaagtagttgtgtatctaaacatatctaaaacatagaaaactaatgtgttgtgctacaatgttacaatgactatgacattgctaggcaataggaattataattttatccttttatggaaccacacttatatatgcggtccatggtggaccaaaacatccttatgtggcatatgactgtatacatgtacacaaaaaatagatgaaagaatgaatatacatcaaaatatttaaaatggttataatgacttaggttacttttatttatcttagtaataataatgatgatagataatacttttatagtgtttactatataaaagacactgttataagtgttctacatactttacatgtattacctaaatgatataaatataactctgacagtaactaatcttatacgttctcttttcttttttttttttttctttttttagacagaatcttgctctaccaggctggagtgcagggtgcaatctcggctcactgcaacctccgcctcccaggttcaaacgattctcatgtctcagcctcctgagtagctgggactacaggcacacaccaccatgcccggctaatttttgtatttttgggtagagatggagttttgccatgttggccaggctgatcttgaactcctggcctcaagtgatctgcctgcctcagcctcccaaagtgctgggattacaggtgtgaaccactgtgctcggcctaatcttacaagttttcaatatttaaagagtgctaactttgttgacaatataaaacatatttgagaaaaagagatataagcatcttatttagaattatgaaaatatcaatagacctacagccgactaaagcttttcttcataagctcttgcctatattgattcgctcctgtgaatatgcattaatttgatttaaataataagtatgtataagaaataacacttttccttaatttttaagaacgttcaacagtttttaatttgaattccaatagtgaaatacatagaaaatataaaattttctgtagtttagccaaattgtttttgtttcaccacagcattctaccaaaatttcttaataacagtaagaaaatgaatgcatacctcctgcagggagaggggagttaggcagtttatgggcatagttacaagtgagaaatttcattggctaccatttacgctaaattcataaaaactgcattcaattctatatatctattttctttacataaaaaaggtttcaattattggccattaaataaaatagccaccattccagaagttgtgtcatgtttatcctttttataccaccatcatattgcctattatatagattgtgtgtgttccattttctgtaatgggccagacagtaagtatttctggctttggagtccatatggtctctatcataactactcatctctgccattgtagcttaaagattatctaggtcaaatgcctaagtgatatagtgttgaaatacaagttatataatataggctgccacaaaaaaaaatttatttggtctaaaaaagatttcatgacttttgtagcagcatgggtggggcatgcaccacttggttaactcggtgtatctttctcctttgcagatctgtccaactcaatggtctaactctaaagatggtggatgatcaaaccttgccacctttaatggaaaaacctctccggccaggaagttcactgggcttgccagctttctcatatagtttttttgtgataagaaatgccaaagttgctgcttgcatctgaaaataaaatatactagtcctgacactgaatttttcaagtatactaagagtaaagcaactcaagttataggaaaggaagcagataccttgcaaagcaactagtgggtgcttgagagacactgggacactgtcagtgctagatttagcacagtattttgatctcgctaggtagaacactgctaataataatagctaataataccttgttccaaatactgcttagcattttgcatgttttacttttatctaaagttttgttttgttttattatttatttatttatttattttgagacagaatctctctctgtcacccaggctggagtgccatggtgcgatcttggctcactgcaactttaagcaattctcctgcctcagcttcctgagtagctgggattataggcgtgtgccaccacgcccagctactttctatattttttgtagagatggagtttcgccatattggccaagctggtctcgaactcctgtcctcgaactcctgtcctcaagtgatccacccgcctcagcctctcaaagtgctgggattacaggtgtgagccaccacacccagcagtgttttatttttgagacagggtatcattctgttgcccaggcttgagtgcagtggtgcaatcatagatcactgcagccttttaactcctgggctcaagtcatcctcctgcttagcctcccaagtagctaggaccacagacacatgccatcacacttggctatttttaaaaaattttttgtagagatggggtctcgctatgttacccaaactggtcctgaactcctggactcaattgatcctcccaccttggccttccaggtgctgggatttctttgggagtacagcatggtacagcaggagatcatttgatgttacctctgtgcagtgttgctagtcagcgaaagactataatacctgtggggacagcgattagccaccacaaccagtctttatttaaagttattaaaaatggctgggcgcagtggctcacacctgtaatcctagcactttgggaggccgaggcagatggatcacctgacgtgaggaatttgagaccagcctggccaacatggtgaaaccccatctctactaaaaaatacaaaaattagctgggtgtggtcctgtagtcccagctacttgggaggctggggcaggagaattacttgaacccaggaggcagaggttgcagtgagccgagattgtgccactgcactccagcctgggtgacagagagagattccatctcaaaaaaacaagttattaaaaatgtatatgaatgctcctaatatggtcaggaagcaaggaagcgaaggatatattatgagttttaagaaggtgcttagctgtatatttatctttcaaaatgtattagaagattttagaattctttccttcatgtgccatctctacaggcacccatcagaaaaagcatactgccgttaccgtgaaactggttgtaaaagagaaactatctatttgcaccttaaaagacagctagattttgctgattttcttctttcggttttctttgtcagcaataatatgtgagaggacagattgttagatatgatagtataaaaaatggttaatgacaattcagaggcgaggagattctgtaaacttaaaattactataaatgaaattgatttgtcaagaggataaattttagaaaacacccaataccttataactgtctgttaatgcttgctttttctctacctttcttccttgtttcagttgggaagcttttggctgcaagtaacagaaactcctaattcaaatggcttaagcaataaggaaatgtatattcccacataactagacgttcaaacaggccaggctccagcacttcagtacgtcaccagggatctgggttcttcccagctctctgctctgccatctttagcgctggcttcattctcagactctggtagcatgatggctgtagctgtttcatgggccccttcaaacctcatagcaaccagaggaagaaaatgagccattttttgagtctccttcatagacttgaataactctttttcagagcttctcacagcaaacctctcctcatgtctcctcatgtcttattgttcagaaatgggtaatgtggccatttcaccagtcactgccaacaacaacgaggttcctataattgtctctgagtaaccctttggaatggagagggtgttggtcagtctacaaactgaacactgcagttctgcgctttttaccagtgaaaaaatgtaattattttcccctcttaaggattaatattcttcaaatgtatgcctgttatggatatagtatctttaaaattttttattttaatagctttaggggtacacactttttgcttacaggggtgaattgtgtagtggtgaagactcggcttttaatgtacttgtcacctgagtgatgtacattgtacccaataggtaatttttcatccattaccctccttccgccctcttcccttctgagtctccaacatcccttataccactgtgtatgttcttgtgtacctacagctaagcttccacttataagtgagaacatgcagtatttggttttccattcctgagttacttcccttaggataacagcccccagttccgtccaagttgctgcaaaatacattattcttctttatggctgagtaatagtccatggtacatatataccacattttctttatccacttatcagttgatggacacttaggttaattccattcaatttcattcaatttaagtatatttgtaaggagctaaagctgaaaattaaattttagatctttcaatactcttaaattttatatgtaagtggtttttatattttcacatttgaaataaagtaatttttataaccttgatattgtatgactattcttttagtaatgtaaagcctacagactcctacatttggaaccactagtgtgttgtttcaccccttgttatactatcaggatcctcga

SEQ ID NO: 43 - DNA, 2396 bp, Mus musculus
tttctagttgcttttagccaatgtcggatcaggtttttcaagcgacaaagagatactgagatcctgggcagaggacatcctagctcggtcagatttgggcaggctcaagtgaccagtgtcttaaggcagaagggagtcggggtagggtctggctgaaccctcaaccggggcttttaactcagggtctagtcctggcgccaaatggatgggacctagaaaaggtgacagagtgcgcaggacaccaggaagctggtcccacccctgcgcggctcccgggcgctccctccccaggcctccgaggatcttggattctggccacctccgcaccctttggatgggtgtggatgatttcaaaagtggacgtgaccgcggcggaggggaaagccagcacggaaatgaaagagagcgaggaggggagggcggggaggggagggcgctagggagggactcccgggaggggtgggagggatggagcgctgtgggagggtactgagtcctggcgccagaggcgaagcaggaccggttgcagggggcttgagccagcgcgccggctgccccagctctcccggcagcgggcggtccagccaggtgggatgctgaggctgctgctgctgtggctctgggggccgctcggtgccctggcccagggcgcccccgcggggaccgcgccgaccgacgacgtggtagacttggagttttacaccaagcggccgctccgaagcgtgagtccctcgttcctgtccatcaccatcgacgccagcctggccaccgacccgcgcttcctcaccttcctgggctctccaaggctccgtgctctggctagaggcttatctcctgcatacttgagatttggcggcacaaagactgacttccttatttttgatccggacaaggaaccgacttccgaagaaagaagttactggaaatctcaagtcaaccatgatatttgcaggtctgagccggtctctgctgcggtgttgaggaaactccaggtggaatggcccttccaggagctgttgctgctccgagagcagtaccaaaaggagttcaagaacagcacctactcaagaagctcagtggacatgctctacagttttgccaagtgctcggggttagacctgatctttggtctaaatgcgttactacgaaccccagacttacggtggaacagctccaacgcccagcttctccttgactactgctcttccaagggttataacatctcctgggaactgggcaatgagcccaacagtttctggaagaaagctcacattctcatcgatgggttgcagttaggagaagactttgtggagttgcataaacttctacaaaggtcagctttccaaaatgcaaaactctatggtcctgacatcggtcagcctcgagggaagacagttaaactgctgaggagtttcctgaaggctggcggagaagtgatcgactctcttacatggcatcactattacttgaatggacgcatcgctaccaaagaagattttctgagctctgatgcgctggacacttttattctctctgtgcaaaaaattctgaaggtcactaaagagatcacacctggcaagaaggtctggttgggagagacgagctcagcttacggtggcggtgcacccttgctgtccaacacctttgcagctggctttatgtggctggataaattgggcctgtcagcccagatgggcatagaagtcgtgatgaggcaggtgttcttcggagcaggcaactaccacttagtggatgaaaactttgagcctttacctgattactggctctctcttctgttcaagaaactggtaggtcccagggtgttactgtcaagagtgaaaggcccagacaggagcaaactccgagtgtatctccactgcactaacgtctatcacccacgatatcaggaaggagatctaactctgtatgtcctgaacctccataatgtcaccaagcacttgaaggtaccgcctccgttgttcaggaaaccagtggatacgtaccttctgaagccttcggggccggatggattactttccaaatctgtccaactgaacggtcaaattctgaagatggtggatgagcagaccctgccagctttgacagaaaaacctctccccgcaggaagtgcactaagcctgcctgccttttcctatggtttttttgtcataagaaatgccaaaatcgctgcttgtatatgaaaataaaaggcatacggtacccctgagacaaaagccgaggggggtgttattcataaaacaaaaccctagtttaggaggccacctccttgccgagttccagagcttcgggagggtggggtacacttcagtattacattcagtgtggtgttctctctaagaagaatactgcaggtggtgacagttaatagcactgtg

SEQ ID NO: 44 - protein, 535 aa, Mus musculus
MLRLLLLWLWGPLGALAQGAPAGTAPTDDVVDLEFYTKRPLRSVSPSFLSITIDASLATDPRFLTFLGSPRLRALARGLSPAYLRFGGTKTDFLIFDPDKEPTSEERSYWKSQVNHDICRSEPVSAAVLRKLQVEWPFQELLLLREQYQKEFKNSTYSRSSVDMLYSFAKCSGLDLIFGLNALLRTPDLRWNSSNAQLLLDYCSSKGYNISWELGNEPNSFWKKAHILIDGLQLGEDFVELHKLLQRSAFQNAKLYGPDIGQPRGKTVKLLRSFLKAGGEVIDSLTWHHYYLNGRIATKEDFLSSDALDTFILSVQKILKVTKEITPGKKVWLGETSSAYGGGAPLLSNTFAAGFMWLDKLGLSAQMGIEVVMRQVFFGAGNYHLVDENFEPLPDYWLSLLFKKLVGPRVLLSRVKGPDRSKLRVYLHCTNVYHPRYQEGDLTLYVLNLHNVTKHLKVPPPLFRKPVDTYLLKPSGPDGLLSKSVQLNGQILKMVDEQTLPALTEKPLPAGSALSLPAFSYGFFVIRNAKIAACI

SEQ ID NO: 45 - DNA, 2396 bp, Mus musculus; identical to SEQ ID NO: 43
  CDS (594)..(2198)

SEQ ID NO: 46 - DNA, 385 bp, Rattus norvegicus
cggccgctgctgctgctgtggctctgggggcggctccgtgccctgacccaaggcactccggcggggaccgcgccgaccaaagacgtggtggacttggagttttacaccaagaggctattccaaagcgtgagtccctcgttcctgtccatcaccatcgacgccagtctggccaccgaccctcggttcctcaccttcctgagctctccacggcttcgagccctgtctagaggcttatctcctgcgtacttgagatttggcggcaccaagactgacttccttatttttgatcccaacaacgaacccacctctgaagaaagaagttactggcaatctcaagacaacaatgatatttgcgggtctgaccgggtctccgctgacgtgttga

SEQ ID NO: 47 - DNA, 541 bp, Rattus norvegicus
  misc_feature (507)..(507): Any nucleotide
aaatcaggacatatccttcacttatttgcctcttggtcatattggaggcatttgtattcatttttaataaccctcaaaatagtgcatgcaaagtgctaagcgtcatttgccacatggtgccattaactgtcaccacctgcagtggtctacttagagaacaccgcactggatgttaacactgaagcgcgtgccccgccctcccgaggctctggatccagcgttgaagcttgccccgccctcccgaggctctggatccagcactggagcatgccccgccctcccgaggctctggagcttgctaaggagtccgctccctaccgctggggttttgctttattcttatgaatgacacccctgaccgctttcgtctcaggggtactgtaatgccttttattttcatatacaagctgcgattttggcatttcttatgacaaaaaacccataggaaaaggcgggcacgcttagtgagcttcctgcggggagaggtttttctgttagagctggcanggtctgctcatcgaccatcttcaggcctcgtgcc

SEQ ID NO: 48 - DNA, 17 bp, Artificial sequence
  Single strand DNA oligonucleotide
ataggcagctgacctga

SEQ ID NO: 49 - DNA, 24 bp, Artificial sequence
  Single strand DNA oligonucleotide
tgacttgagattgccagtaacttc

SEQ ID NO: 50 - DNA, 24 bp, Artificial sequence
  Single strand DNA oligonucleotide
ctgtccaactcaatggtctaactc

SEQ ID NO: 51 - DNA, 20 bp, Artificial sequence
  Single strand DNA oligonucleotide
tctagagcctctgctaacca

(19) United States
(12) Patent Application Publication
(10) Pub. No.: US 2003/0217375 A1
(43) Pub. Date: Nov. 20, 2003

(54) Transgenic animals expressing heparanase and uses thereof

(76) Inventors: Eyal Zcharia, Jerusalem (IL); Israel Vlodavsky, Mevaseret Zion (IL); Shula Metzger, Jerusalem (IL); Iris Pecker, Rishon LeZion (IL); Neta Ilan, Rehovot (IL); Tova Chajek-Shaul, Jerusalem (IL); Orit Goldshmidt, Jerusalem (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(21) Appl. No.: 10/371,218

(22) Filed: Feb. 24, 2003

Related U.S. Application Data
(63) Continuation-in-part of application No. 09/988,113, filed on Nov. 19, 2001.
Application No. 09/988,113 is a continuation of application No. 09/776,874, filed on Feb. 6, 2001.
Application No. 09/776,874 is a continuation of application No. 09/258,892, filed on Mar. 1, 1999, now abandoned.
Application No. 09/258,892 is a continuation-in-part of application No. PCT/US98/17954, filed on Aug. 31, 1998.

(51) Int. Cl. A01K 67/027; C12N 9/24

(52) U.S. Cl. 800/7; 800/14; 800/19; 435/200

(57) ABSTRACT

A transgenic non-human animal expressing heparanase from a transgene, methods for its preparation, compositions-of-matter derived therefrom and uses thereof.

This is a continuation-in-part of U.S. patent application Ser. No. 09/988,113, filed Feb. 6, 2001, which is a continuation of U.S. patent application Ser. No. 09/776,874, filed Feb. 6, 2001, which is a continuation of U.S. patent application Ser. No. 09/258,892, filed Mar. 1, 1999, now abandoned, which is a continuation-in-part of PCT/US98/17954, filed Aug. 31, 1998, which claims priority from U.S. patent application Ser. No. 09/109,386, filed Jul. 2, 1998, now abandoned, which is a continuation-in-part of U.S. patent application Ser. No. 08/922,170, filed Sep. 2, 1997, now, U.S. Pat. No. 5,968,822, issued Oct. 19, 1999. This application is also a continuation-in-part of U.S. patent application Ser. No. 09/864,321, filed May 25, 2001.

### FIELD AND BACKGROUND OF THE INVENTION

The present invention relates to transgenic animals expressing heparanase and to the uses thereof as a model for human disease and for the commercial production of heparanase.

Glycosaminoglycans (GAGs):

GAGs are polymers of repeated disaccharide units consisting of uronic acid and a hexosamine. Biosynthesis of GAGs except hyaluronic acid is initiated from a core protein. Proteoglycans may contain several GAG side chains from similar or different families. GAGs are synthesized as homopolymers which may subsequently be modified by N-deacetylation and N-sulfation, followed by C5-epimerization of glucuronic acid to iduronic acid and O-sulfation. The chemical composition of GAGs from various tissues varies to a great extent.

The natural metabolism of GAGs in animals is carried out by hydrolysis. Generally, the GAGs are degraded in a two step procedure. First the proteoglycans are internalized in endosomes, where initial depolymerization of the GAG chain takes place. This step is mainly hydrolytic and yields oligosaccharides. Further degradation is carried out following fusion with lysosome, where desulfation and exolytic depolymerization to monosaccharides take place (42).

The only GAG degrading endolytic enzymes characterized so far in animals are the hyaluronidases. The hyaluronidases are a family of 1-4 endoglucosaminidases that depolymerize hyaluronic acid and chondroitin sulfate. The cDNAs encoding sperm associated PH-20 (Hyal3), and the lysosomal hyaluronidases Hyal 1 and Hyal 2 were cloned and published (27). These enzymes share an overall homology of 40% and have different tissue specificities, cellular localizations and pH optima for activity.

Exolytic hydrolases are better characterized, among which are beta-glucuronidase, alpha-L-iduronidase and beta-N-acetylglucosaminidase. In addition to hydrolysis of the glycosidic bond of the polysaccharide chain, GAG degradation involves desulfation, which is catalyzed by several lysosomal sulfatases such as N-acetylgalactosamine-4-sulfatase, iduronate-2-sulfatase and heparin sulfamidase. Deficiency in any of lysosomal GAG degrading enzymes results in a lysosomal storage disease known as mucopolysaccharidosis.

Glycosyl Hydrolases:

Glycosyl hydrolases are a widespread group of enzymes that hydrolyze the o-glycosidic bond between two or more carbohydrates or between a carbohydrate and a noncarbohydrate moiety. The enzymatic hydrolysis of glycosidic bond occurs by one or two major mechanisms leading to overall retention or inversion of the anomeric configuration. In both mechanisms, catalysis involves a proton donor and a nucleophile. Glycosyl hydrolyses have been classified into 58 families based on amino acid similarities. The glycosyl hydrolyses from families 1, 2, 5, 10, 17, 30, 35, 39 and 42 act on a large variety of substrates, however, they all hydrolyze the glycosidic bond in a general acid catalysis mechanism, with retention of the anomeric configuration. The mechanism involves two glutamic acid residues, which serve as the proton donor and the nucleophile, with an asparagine, which always precedes the proton donor. Analyses of a set of known 3D structures from this group of enzymes revealed that their catalytic domains, despite the low level of sequence identity, adopt a similar (alpha/beta) 8 fold with the proton donor and the nucleophile located at the C-terminal ends of strands beta 4 and beta 7, respectively. Mutations in the functional conserved amino acids of lysosomal glycosyl hydrolases were identified in lysosomal storage diseases.

Lysosomal glycosyl hydrolases including beta-glucuronidase, beta-mannosidase, beta-glucocerebrosidase, beta-galactosidase and alpha-L-iduronidase, are all exo-glycosyl hydrolases, belong to the GH-A clan and share a similar catalytic site. However, many endo-glucanases from various organisms, such as bacterial and fungal xylenases and cellulases share this catalytic domain (1).

Heparan Sulfate Proteoglycans (HSPGs):

HSPGs are ubiquitous macromolecules associated with the cell surface and extracellular matrix (ECM) of a wide range of cells of vertebrate and invertebrate tissues (3-7). The basic HSPG structure consists of a protein core to which several linear heparan sulfate chains are covalently attached. The polysaccharide chains are typically composed of repeating hexuronic and D-glucosamine disaccharide units that are substituted to a varying extent with N- and O-linked sulfate moieties and N-linked acetyl groups (3-7). Studies on the involvement of ECM molecules in cell attachment, growth and differentiation revealed a central role of HSPGs in embryonic morphogenesis, angiogenesis, metastasis, neurite outgrowth and tissue repair (3-7). The heparan sulfate (HS) chains, which are unique in their ability to bind a multitude of proteins, ensure that a wide variety of effector molecules cling to the cell surface (6-8). HSPGs are also prominent components of blood vessels (5). In large vessels they are concentrated mostly in the intima and inner media, whereas in capillaries they are found mainly in the subendothelial basement membrane where they support proliferating and migrating endothelial cells and stabilize the structure of the capillary wall. The ability of HSPGs to interact with ECM macromolecules such as collagen, laminin and fibronectin, and with different attachment sites on plasma membranes suggests a key role for this proteoglycan in the self-assembly and insolubility of ECM components, as well as in cell adhesion and locomotion. Cleavage of HS may therefore result in disassembly of the subendothelial ECM and hence may play a decisive role in extravasation of normal and malignant blood-borne cells (9-11). HS catabolism is observed in inflammation, wound repair, diabetes, and cancer metastasis, suggesting that enzymes, which degrade HS, play important roles in pathologic processes.

Heparanase:

Heparanase is a glycosylated enzyme that is involved in the catabolism of certain glycosaminoglycans. It is an endoglucuronidase that cleaves heparan sulfate at specific intrachain sites (12-15). Interaction of T and B lymphocytes, platelets, granulocytes, macrophages and mast cells with the subendothelial extracellular matrix (ECM) is associated with degradation of heparan sulfate by heparanase activity (16). Placental heparanase acts as an adhesion molecule or as a degradative enzyme depending on the pH of the microenvironment (17).

Heparanase is released from intracellular compartments (e.g., lysosomes, specific granules) in response to various activation signals (e.g., thrombin, calcium ionophores, immune complexes, antigens and mitogens), suggesting its regulated involvement in inflammation and cellular immunity responses (16).

It was also demonstrated that heparanase can be readily released from human neutrophils by 60 minutes incubation at 4° C. in the absence of added stimuli (18).

Gelatinase, another ECM degrading enzyme, which is found in tertiary granules of human neutrophils with heparanase, is secreted from the neutrophils in response to phorbol 12-myristate 13-acetate (PMA) treatment (19-20).

In contrast, various tumor cells appear to express and secrete heparanase in a constitutive manner in correlation with their metastatic potential (21).

Degradation of heparan sulfate by heparanase results in the release of heparin-binding growth factors, enzymes and plasma proteins that are sequestered by heparan sulfate in basement membranes, extracellular matrices and cell surfaces (22-23).

Heparanase activity has been described in a number of cell types including cultured skin fibroblasts, human neutrophils, activated rat T-lymphocytes, normal and neoplastic murine B-lymphocytes, human monocytes and human umbilical vein endothelial cells, SK hepatoma cells, human placenta and human platelets.

Procedures for purification of natural heparanase were reported for SK hepatoma cells and human placenta (U.S. Pat. No. 5,362,641) and for human platelets derived enzymes (53).

Involvement of Heparanase in Tumor Cell Invasion and Metastasis:

Circulating tumor cells arrested in the capillary beds often attach at or near the intercellular junctions between adjacent endothelial cells. Such attachment of the metastatic cells is followed by rupture of the junctions, retraction of the endothelial cell borders and migration through the breach in the endothelium toward the exposed underlying base membrane (BM) (24). Once located between endothelial cells and the BM, the invading cells must degrade the subendothelial glycoproteins and proteoglycans of the BM in order to migrate out of the vascular compartment. Several cellular enzymes (e.g., collagenase IV, plasminogen activator, cathepsin B, elastase, etc.) are thought to be involved in degradation of BM (25). Among these enzymes is heparanase that cleaves HS at specific intrachain sites (16, 11). Expression of a HS degrading heparanase was found to correlate with the metastatic potential of mouse lymphoma (26), fibrosarcoma and melanoma (21) cells. Moreover, elevated levels of heparanase were detected in sera from metastatic tumor bearing animals and melanoma patients (21) and in tumor biopsies of cancer patients (12).

The inhibitory effect of various non-anticoagulant species of heparin on heparanase was examined in view of their potential use in preventing extravasation of blood-borne cells. Treatment of experimental animals with heparanase inhibitors markedly reduced (>90%) the incidence of lung metastases induced by B16 melanoma, Lewis lung carcinoma and mammary adenocarcinoma cells (12, 13, 28). Heparin fractions with high and low affinity to anti-thrombin III exhibited a comparable high anti-metastatic activity, indicating that the heparanase inhibiting activity of heparin, rather than its anticoagulant activity, plays a role in the anti-metastatic properties of the polysaccharide (12).

The direct role of heparanase in cancer metastasis was demonstrated by two experimental systems. The murine T-lymphoma cell line Eb has no detectable heparanase activity. Whether the introduction of the hpa gene into Eb cells would confer a metastatic behavior on these cells was investigated. To this purpose, Eb cells were transfected with a full length human hpa cDNA. Stable transfected cells showed high expression of the heparanase mRNA and enzyme activity. These hpa and mock transfected Eb cells were injected subcutaneously into DBA/2 mice and mice were tested for survival time and liver metastases. All mice (n=20) injected with mock transfected cells survived during the first 4 weeks of the experiment, while 50% mortality was observed in mice inoculated with Eb cells transfected with the hpa cDNA. The liver of mice inoculated with hpa transfected cells was infiltrated with numerous Eb lymphoma cells, as was evident both by macroscopic evaluation of the liver surface and microscopic examination of tissue sections. In contrast, metastatic lesions could not be detected by gross examination of the liver of mice inoculated with mock transfected control Eb cells. Few or no lymphoma cells were found to infiltrate the liver tissue. In a different model of tumor metastasis, transient transfection of the heparanase gene into low metastatic B16-F1 mouse melanoma cells followed by intravenous inoculation, resulted in a 4- to 5-fold increase in lung metastases.

Finally, heparanase externally adhered to B16-F1 melanoma cells increased the level of lung metastases in C57BL mice as compared to control mice (see U.S. patent application Ser. No. 09/260,037, which is incorporated herein by reference).

Possible Involvement of Heparanase in Tumor Angiogenesis:

Fibroblast growth factors are a family of structurally related polypeptides characterized by high affinity to heparin (29). They are highly mitogenic for vascular endothelial cells and are among the most potent inducers of neovascularization (29-30). Basic fibroblast growth factor (bFGF) has been extracted from a subendothelial ECM produced in vitro (31) and from basement membranes of the cornea (32), suggesting that ECM may serve as a reservoir for bFGF. Immunohistochemical staining revealed the localization of bFGF in basement membranes of diverse tissues and blood vessels (23). Despite the ubiquitous presence of bFGF in normal tissues, endothelial cell proliferation in these tissues is usually very low, suggesting that bFGF is somehow sequestered from its site of action. Studies on the interaction of bFGF with ECM revealed that bFGF binds to HSPG in the ECM and can be released in an active form by HS degrading enzymes (33, 32, 34). It was demonstrated that heparanase activity expressed by platelets, mast cells, neutrophils, and lymphoma cells is involved in release of active bFGF from ECM and basement membranes (35), suggesting that heparanase activity may not only function in cell migration and invasion, but may also elicit an indirect neovascular response. These results suggest that the ECM HSPG provides a natural storage depot for bFGF and possibly other heparin-binding growth promoting factors (36, 37). Displacement of bFGF from its storage within basement membranes and ECM may therefore provide a novel mechanism for induction of neovascularization in normal and pathological situations.

Recent studies indicate that heparin and HS are involved in binding of bFGF to high affinity cell surface receptors and in bFGF cell signaling (38, 39). Moreover, the size of HS required for optimal effect was similar to that of HS fragments released by heparanase (40). Similar results were obtained with vascular endothelial cells growth factor (VEGF) (41), suggesting the operation of a dual receptor mechanism involving HS in cell interaction with heparin-binding growth factors. It is therefore proposed that restriction of endothelial cell growth factors in ECM prevents their systemic action on the vascular endothelium, thus maintaining a very low rate of endothelial cells turnover and vessel growth. On the other hand, release of bFGF from storage in ECM as a complex with HS fragment, may elicit localized endothelial cell proliferation and neovascularization in processes such as wound healing, inflammation and tumor development (36, 37).

The Involvement of Heparanase in Other Physiological Processes and its Potential Therapeutic Applications:

Apart from its involvement in tumor cell metastasis, inflammation and autoimmunity, mammalian heparanase may be applied to modulate bioavailability of heparin-binding growth factors; cellular responses to heparin-binding growth factors (e.g., bFGF, VEGF) and cytokines (IL-8) (44, 41); cell interaction with plasma lipoproteins (49); cellular susceptibility to certain viral and some bacterial and protozoa infections (45-47); and disintegration of amyloid plaques (48).

Viral infection: The presence of heparan sulfate on cell surfaces have been shown to be the principal requirement for the binding of Herpes Simplex (45) and Dengue (46) viruses to cells and for subsequent infection of the cells. Removal of the cell surface heparan sulfate by heparanase may therefore abolish virus infection. In fact, treatment of cells with bacterial heparitinase (degrading heparan sulfate) or heparinase (degrading heparan) reduced the binding of two related animal herpes viruses to cells and rendered the cells at least partially resistant to virus infection (45). There are some indications that the cell surface heparan sulfate is also involved in HIV infection (47).

Neurodegenerative diseases: Heparan sulfate proteoglycans were identified in the prion protein amyloid plaques of Genstmann-Straussler Syndrome, Creutzfeldt-Jakob disease and Scrape (48). Heparanase may disintegrate these amyloid plaques, which are also thought to play a role in the pathogenesis of Alzheimer's disease.

Restenosis and atherosclerosis: Proliferation of arterial smooth muscle cells (SMCs) in response to endothelial injury and accumulation of cholesterol rich lipoproteins are basic events in the pathogenesis of atherosclerosis and restenosis (50). Apart from its involvement in SMC proliferation as a low affinity receptor for heparin-binding growth factors, HS is also involved in lipoprotein binding, retention and uptake (51). It was demonstrated that HSPG and lipoprotein lipase participate in a novel catabolic pathway that may allow substantial cellular and interstitial accumulation of cholesterol rich lipoproteins (49). The latter pathway is expected to be highly atherogenic by promoting accumulation of apoB and apoE rich lipoproteins (e.g., LDL, VLDL, chylomicrons), independent of feed back inhibition by the cellular cholesterol content. Removal of SMC HS by heparanase is therefore expected to inhibit both SMC proliferation and lipid accumulation and thus may halt the progression of restenosis and atherosclerosis.

Pulmonary diseases: The data obtained from the literature suggests a possible role for GAGs degrading enzymes, such as, but not limited to, heparanases, connective tissue activating peptide, heparinases, hyluronidases, sulfatases and chondroitinases, in reducing the viscosity of sinuses and airway secretions with associated implications on curtailing the rate of infection and inflammation. The sputum from CF patients contains at least 3% GAGs, thus contributing to its volume and viscous properties. It was shown that heparanase reduces the viscosity of sputum of Cystic fibrosis (CF) patients (see, U.S. Pat. No. 6,153,187). Recombinant heparanase has been shown to reduce viscosity of sputum of CF patients (see, (see, U.S. Pat. No. 6,153,187).

Heparanase and/or heparanase inhibitors may thus prove useful for treating conditions such as wound healing, angiogenesis, restenosis, atherosclerosis, inflammation, neurodegenerative diseases and viral infections. Mammalian heparanase can be used to neutralize plasma heparin, as a potential replacement of protamine.

Transgenic Non-Human Models of Disease: The advantages, and validity of studying disease processes in non-human models has been long recognized, and such research is, for example, a requisite stage in development of all drugs and therapies for use in humans. Among the preferred species commonly used for such studies, the mouse is clearly the mammal most extensively classified and is often the model of choice (for an extensive review of the field see Bockamp et al, Physiol Genomics 2002;11:115-132).

Even before the widespread application of transgenic technology, many large breeders of laboratory animals invested significant effort and expense in the establishment, using traditional breeding techniques, of mouse strains bearing phenotypes useful for the study of specific diseases and/or treatments. Jackson Laboratories, for example (www.jax.org) offer over 300 stock strains of inbred, hybrid, wild-derived inbred and recombinant inbred mice with well-defined phenotypic characteristics.

Using genomic engineering technology, however, specific alterations in genotype, and their phenotypic effects can now be studied with greater precision at a fraction of the cost and time required for breeding stock strains. For example, Jackson Laboratories today offer thousands of stock strains of transgenic mouse models for investigation in the fields of, inter alia, Cancer research, Diabetes and Obesity, Cardiovascular Disease, Immunology and Neurobiology.

Transgenic models of human disease are often produced by introduction into mice of disease-associated transgenes bearing previously identified alterations of coding and regulatory sequences, for the comparison of their phenotypic effects with known characteristics of the human disease. For example, the Oncomouse™ (DuPont Nemours, Inc.) strains, bearing a variety of oncogene mutations, have become indispensible tools for Cancer research.

Transgenic mouse models can also be engineered to express proteins known to be associated with human disease in conditions having unclear etiology, providing researchers with tools to investigate disease processes, complex interactions with multiple pathogenic factors, combinations of risk factors and susceptibility to disease. Examples include mouse models of Alzheimer's disease expressing amyloid protein (U.S. Pat. No. 6,509,515 to Hsiao et al) and tau filaments (Tatebayashi Y et al. PNAS USA 2002;99:13896-901); mouse models of Diabetes Mellitus expressing human islet amyloid polypeptide (Janson et al PNAS USA 1996;93:7283-88); mouse models of colorectal cancer expressing human carcinoembryonic antigen (CEA) (Wilkinson R W et al PNAS USA 2001;98:10256-60); mouse models of Duchenne's Muscular Dystrophy overexpressing human caveolin-3 (Galbiati F et al. PNAS USA 2000;97:9684-94) and mouse models of skin disease and tumorigenesis expressing human collagenase (Darmiento, J et al Mol Cell Biol 1995;15:5732-39). These transgenic mouse models also provide important tools for evaluation of specific effects of therapies, screening of pharmaceuticals and development of diagnostic methodologies. The demonstrated involvement of heparanase in immune response, inflammation, malignancy, metastasis, angiogenesis, tumorigenesis, viral infection, atherogenesis, pulmonary disease and other conditions, as detailed hereinabove, creates a strong need for a transgenic model of human heparanse over- or under-expression.

There is, thus, a widely recognized need for, and it would be highly advantageous to have, transgenic animals producing heparanase so as to efficiently produce commercial quantities of this enzyme. Such transgenic animals would also find uses as models for human disease associated with impaired heparanase expression, such as, for example, metastasis.

### SUMMARY OF THE INVENTION

According to one aspect of the present invention there is provided a transgenic non-human animal whose genome comprises an exogenous polynucleotide sequence integrated into the genome, the exogenous polynucleotide sequence including a promoter active in tissues of the non-human, and a region encoding a human heparanase, wherein the promoter and the region encoding human heparanase are operably linked in the exogenous polynucleotide such that human heparanase is expressed in at least a portion of the cells of the non-human animal.

According to further features in the described preferred embodiments the transgenic non-human animal being homozygous or heterozygous for the exogenous polynucleotide sequence.

According to still further features in the described preferred embodiments the transgenic non-human animal having a single locus or at least two loci each harboring the exogenous polynucleotide sequence.

According to yet further features in the described preferred embodiments the human heparanase is genetically modified to be cleavable into an active form via a protease.

According to still further features in the described preferred embodiments the heparanase is processed by an endogenous protease of the non-human animal into an active form.

According to yet further features in the described preferred embodiments the region of the exogenous polynucleotide sequence encodes an active form of heparanase.

According to still further features in the described preferred embodiments the transgenic non-human animal is a mammal or an avian.

According to further features in the described preferred embodiments the exogenous polynucleotide sequence includes a tissue specific promoter for directing expression of the heparanase in a tissue specific manner. Accordingly, the promoter is a constitutive promoter for directing expression of the heparanase in constitutive manner or an inducible promoter for directing expression of the heparanase in an inducible manner.

According to further features in the described preferred embodiments the promoter is selected from the group consisting of beta-lactoglobulin promoter, Rb promoter, preproendothelin-1 promoter, beta-actin promoter, TetO promoter, metallothionein promoter, whey acidic protein (WAP) promoter, casein promoter and lactalbumin promoter.

According to still further features in the described preferred embodiments the promoter is selected from the group consisting of chicken lyzozyme promoter, cytomegalovirus promoter and chicken immunoglobulin promoter.

According to yet further features in the described preferred embodiments the heparanase is expressed in, and secreted by, cells of mammary glands of the transgenic non-human mammal.

According to still further features in the described preferred embodiments the heparanase is expressed in, and secreted by, egg producing cells of the transgenic femal avian.

According to a further aspect of the present invention there are provided sex cells, semen and embryos derived from the transgenic non-human animal of the invention.

According to a further aspect of the present invention there is provided a composition of matter comprising milk derived from a non-human transgenic mammal, the milk having detectable human heparanase activity.

According to a still further aspect of the present invention there is provided a composition of matter comprising egg yolk and/or white from a transgenic avian, the egg yolk and/or white having detectable human heparanase activity.

According to a further aspect of the present invention there is provided a method of producing recombinant human heparanase, the method comprising the steps of (a) obtaining a transgenic non-human mammal having mammary glands, whose genome comprises an exogenous polynucleotide sequence integrated into the genome, the exogenous polynucleotide sequence including a promoter active in tissues of the non-human mammal, and a region encoding a human heparanase, wherein the promoter and the region encoding human heparanase are operably linked in the exogenous polynucleotide such that the recombinant human heparanase is secreted into milk being produced by the mammary glands, (b) milking the non-human mammal so as to obtain milk containing the recombinant human heparanase, and (c) purifying the recombinant human heparanase from the milk.

According to further features in the described preferred embodiments the promoter active in tissues of the non-human mammal is a milk protein gene promoter.

According to still further features in the described preferred embodiments the milk protein gene promoter is selected from the group consisting of beta-lactoglobulin promoter, Rb promoter, preproendothelin-1 promoter, whey acidic protein (WAP) promoter, casein promoter and lactalbumin promoter.

According to a further aspect of the present invention there is provided a method of producing recombinant human heparanase, the method comprising the steps of (a) obtaining a transgenic female avian having egg producing cells whose genome comprises an exogenous polynucleotide sequence integrated into the genome, the exogenous polynucleotide sequence including a promoter active in tissues of the transgenic female avian, and a region encoding a human heparanase, wherein the promoter and the region encoding human heparanase are operably linked in the exogenous polynucleotide such that the recombinant human heparanase is secreted into eggs being produced by the egg producing cells, (b) collecting eggs laid by the transgenic female avian so as to obtain eggs containing the recombinant human heparanase and (c) purifying the recombinant human heparanase from the eggs.

According to still further features in the described preferred embodiments the promoter active in tissues of the transgenic female avian is an egg protein gene promoter.

According to still further features in the described preferred embodiments the egg protein gene promoter is selected from the group consisting of chicken lyzozyme promoter and chicken immunoglobulin promoter.

The present invention successfully addresses the shortcomings of the presently known configurations by providing transgenic animals expressing heparanase which can be used as animal models and/or for commercial production of recombinant heparanase.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 presents nucleotide sequence and deduced amino acid sequence of hpa cDNA. A single nucleotide difference at position 799 (A to T) between the EST (Expressed Sequence Tag) and the PCR amplified cDNA (reverse transcribed RNA) and the resulting amino acid substitution (Tyr to Phe) are indicated above and below the substituted unit, respectively. Cysteine residues and the poly adenylation consensus sequence are underlined. The asterisk denotes the stop codon TGA.

FIG. 2 demonstrates degradation of soluble sulfate labeled HSPG substrate by lysates of High Five cells infected with pFhpa2 virus. Lysates of High Five cells that were infected with pFhpa2 virus (•) or control pF2 virus (□) were incubated (18 h, 37° C.) with sulfate labeled ECM-derived soluble HSPG (peak I). The incubation medium was then subjected to gel filtration on Sepharose 6B. Low molecular weight HS degradation fragments (peak II) were produced only during incubation with the pFhpa2 infected cells, but there was no degradation of the HSPG substrate (⋄) by lysates of pF2 infected cells.

FIGS. 3a-b demonstrate degradation of soluble sulfate labeled HSPG substrate by the culture medium of pFhpa2 and pFhpa4 infected cells. Culture media of High Five cells infected with pFhpa2 (3a) or pFhpa4 (3b) viruses (•), or with control viruses (□) were incubated (18 h, 37° C.) with sulfate labeled ECM-derived soluble HSPG (peak I, ⋄). The incubation media were then subjected to gel filtration on Sepharose 6B. Low molecular weight HS degradation fragments (peak II) were produced only during incubation with the hpa gene containing viruses. There was no degradation of the HSPG substrate by the culture medium of cells infected with control viruses.

FIG. 4 presents size fractionation of heparanase activity expressed by pFhpa2 infected cells. Culture medium of pFhpa2 infected High Five cells was applied onto a 50 kDa cut-off membrane. Heparanase activity (conversion of the peak I substrate, (⋄) into peak II HS degradation fragments) was found in the high (>50 kDa) (•), but not low (<50 kDa) (∘) molecular weight compartment.

FIGS. 5a-b demonstrate the effect of heparin on heparanase activity expressed by pFhpa2 and pFhpa4 infected High Five cells. Culture media of pFhpa2 (5a) and pFhpa4 (5b) infected High Five cells were incubated (18 h, 37° C.) with sulfate labeled ECM-derived soluble HSPG (peak I, ⋄) in the absence (•) or presence (Δ) of 10 μg/ml heparin. Production of low molecular weight HS degradation fragments was completely abolished in the presence of heparin, a potent inhibitor of heparanase activity (6, 7).

FIGS. 6a-b demonstrate degradation of sulfate labeled intact ECM by virus infected High Five and Sf21 cells. High Five (6a) and Sf21 (6b) cells were plated on sulfate labeled ECM and infected (48 h, 28° C.) with pFhpa4 (•) or control pF1 (□) viruses. Control non-infected Sf21 cells (R) were plated on the labeled ECM as well. The pH of the cultured medium was adjusted to 6.0-6.2 followed by 24 h incubation at 37° C. Sulfate labeled material released into the incubation medium was analyzed by gel filtration on Sepharose 6B. HS degradation fragments were produced only by cells infected with the hpa containing virus.

FIGS. 7a-b demonstrate degradation of sulfate labeled intact ECM by virus infected cells. High Five (7a) and Sf21 (7b) cells were plated on sulfate labeled ECM and infected (48 h, 28° C.) with pFhpa4 (•) or control pF1 (□) viruses. Control non-infected Sf21 cells (R) were plate on labeled ECM as well. The pH of the cultured medium was adjusted to 6.0-6.2, followed by 48 h incubation at 28° C. Sulfate labeled degradation fragments released into the incubation medium was analyzed by gel filtration on Sepharose 6B. HS degradation fragments were produced only by cells infected with the hpa containing virus.

FIGS. 8a-b demonstrate degradation of sulfate labeled intact ECM by the culture medium of pFhpa4 infected cells. Culture media of High Five (8a) and Sf21 (8b) cells that were infected with pFhpa4 (•) or control pF1 (□) viruses were incubated (48 h, 37° C., pH 6.0) with intact sulfate labeled ECM. The ECM was also incubated with the culture medium of control non-infected Sf21 cells (R). Sulfate labeled material released into the reaction mixture was subjected to gel filtration analysis. Heparanase activity was detected only in the culture medium of pFhpa4 infected cells.

FIGS. 9a-b demonstrate the effect of heparin on heparanase activity in the culture medium of pFhpa4 infected cells. Sulfate labeled ECM was incubated (24 h, 37° C., pH 6.0) with culture medium of pFhpa4 infected High Five (9a) and Sf21 (9b) cells in the absence (•) or presence (V) of 10 μg/ml heparin. Sulfate labeled material released into the incubation medium was subjected to gel filtration on Sepharose 6B. Heparanase activity (production of peak II HS degradation fragments) was completely inhibited in the presence of heparin.

FIGS. 10a-b demonstrate purification of recombinant heparanase on heparin-Sepharose. Culture medium of Sf21 cells infected with pFhpa4 virus was subjected to heparin-Sepharose chromatography. Elution of fractions was performed with 0.35-2 M NaCl gradient (⋄). Heparanase activity in the eluted fractions is demonstrated in FIG. 10a (•). Fractions 15-28 were subjected to 15% SDS-polyacrylamide gel electrophoresis followed by silver nitrate staining. A correlation is demonstrated between a major protein band (MW ˜63,000) in fractions 19-24 and heparanase activity.

FIGS. 11a-b demonstrate purification of recombinant heparanase on a Superdex 75 gel filtration column. Active fractions eluted from heparin-Sepharose (FIG. 10a) were pooled, concentrated and applied onto Superdex 75 FPLC column. Fractions were collected and aliquots of each fraction were tested for heparanase activity (c, FIG. 11a) and analyzed by SDS-polyacrylamide gel electrophoresis followed by silver nitrate staining (FIG. 11b). A correlation is seen between the appearance of a major protein band (MW ˜63,000) in fractions 4-7 and heparanase activity.

FIGS. 12a-e demonstrate expression of the hpa gene by RT-PCR with total RNA from human embryonal tissues (12a), human extra-embryonal tissues (12b) and cell lines from different origins (12c-e). RT-PCR products using hpa specific primers (I), primers for GAPDH housekeeping gene (II), and control reactions without reverse transcriptase demonstrating absence of genomic DNA or other contamination in RNA samples (III). M-DNA molecular weight marker VI (Boehringer Mannheim). For 12a: lane 1—neutrophil cells (adult), lane 2—muscle, lane 3—thymus, lane 4—heart, lane 5—adrenal. For 12b: lane 1—kidney, lane 2—placenta (8 weeks), lane 3—placenta (11 weeks), lanes 4-7—mole (complete hydatidiform mole), lane 8—cytotrophoblast cells (freshly isolated), lane 9—cytotrophoblast cells (1.5 h in vitro), lane 10—cytotrophoblast cells (6 h in vitro), lane 11—cytotrophoblast cells (18 h in vitro), lane 12—cytotrophoblast cells (48 h in vitro). For 12c: lane 1—JAR bladder cell line, lane 2—NCITT testicular tumor cell line, lane 3—SW-480 human hepatoma cell line, lane 4—HTR (cytotrophoblasts transformed by SV40), lane 5—HPTLP-I hepatocellular carcinoma cell line, lane 6—EJ-28 bladder carcinoma cell line. For 12d: lane 1—SK-hep-1 human hepatoma cell line, lane 2—DAMI human megakaryocytic cell line, lane 3—DAMI cell line+PMA, lane 4—CHRF cell line+PMA, lane 5—CHRF cell line. For 12e: lane 1—ABAE bovine aortic endothelial cells, lane 2—1063 human ovarian cell line, lane 3—human breast carcinoma MDA435 cell line, lane 4—human breast carcinoma MDA231 cell line.

FIG. 13 presents a comparison between nucleotide sequences of the human hpa and a mouse EST cDNA fragment (SEQ ID NO:12) which is 80% homologous to the 3′ end (starting at nucleotide 1066 of SEQ ID NO:9) of the human hpa. The aligned termination codons are underlined.

FIG. 14 demonstrates the chromosomal localization of the hpa gene. PCR products of DNA derived from somatic cell hybrids and of genomic DNA of hamster, mouse and human of were separated on 0.7% agarose gel following amplification with hpa specific primers. Lane 1—Lambda DNA digested with BstEII, lane 2—no DNA control, lanes 3-29, PCR amplification products. Lanes 3-5—human, mouse and hamster genomic DNA, respectively. Lanes 6-29, human monochromosomal somatic cell hybrids representing chromosomes 1-22 and X and Y, respectively. Lane 30—Lambda DNA digested with BstEII. An amplification product of approximately 2.8 Kb is observed only in lanes 5 and 9, representing human genomic DNA and DNA derived from cell hybrid carrying human chromosome 4, respectively. These results demonstrate that the hpa gene is localized in human chromosome 4.

FIG. 15 demonstrates the genomic exon-intron structure of the human hpa locus (top) and the relative positions of the lambda clones used as sequencing templates to sequence the locus (below). The vertical rectangles represent exons (E) and the horizontal lines therebetween represent introns (I), upstream (U) and downstream (D) regions. Continuous lines represent DNA fragments, which were used for sequence analysis. The discontinuous line in lambda 6 represent a region, which overlaps with lambda 8 and hence was not analyzed. The plasmid contains a PCR product, which bridges the gap between L3 and L6.

FIG. 16 presents the nucleotide sequence of the genomic region of the hpa gene. Exon sequences appear in upper case and intron sequences in lower case. The deduced amino acid sequence of the exons is printed below the nucleotide sequence. Two predicted transcription start sites are shown in bold.

FIG. 17 presents an alignment of the amino acid sequences of human heparanase, mouse and partial sequences of rat homologues. The human and the mouse sequences were determined by sequence analysis of the isolated cDNAs. The rat sequence is derived from two different EST clones, which represent two different regions (5′ and 3′) of the rat hpa cDNA. The human sequence and the amino acids in the mouse and rat homologues, which are identical to the human sequence, appear in bold.

FIG. 18 presents a heparanase Zoo blot. Ten micrograms of genomic DNA from various sources were digested with EcoRI and separated on 0.7% agarose—TBE gel. Following electrophoresis, the was gel treated with HCl and than with NaOH and the DNA fragments were downward transferred to a nylon membrane (Hybond N+, Amersham) with 0.4 N NaOH. The membrane was hybridized with a 1.6 Kb DNA probe that contained the entire hpa cDNA. Lane order: H—Human; M—Mouse; Rt—Rat; P—Pig; Cw—Cow; Hr—Horse; S—Sheep; Rb—Rabbit; D—Dog; Ch—Chicken; F—Fish. Size markers (Lambda BsteII) are shown on the left

FIG. 19 demonstrates the secondary structure prediction for heparanase performed using the PHD server—Profile network Prediction Heidelberg. H—helix, E—extended (beta strand), The glutamic acid predicted as the proton donor is marked by asterisk and the possible nucleophiles are underlined.

FIGS. 20A-Div demonstrate the expression of the heparanase protein in various tissues of homozygous transgenic mice overexpressing the human hpa gene. 20A—Western blot analysis; 20Bi-iii—Heparanase activity (wild=wild type control mice; transg.=transgenic mice); 20Ci-iv—Immunohistochemistry of colon and heart tissues (20Ci and 20Ciii—transgenic mice, 20Cii and 20Civ—control mice). Western analysis and immunohistochemistry were performed using the anti heparanase monoclonal antibody HP-130.

FIGS. 21A-D show morphological appearance of mammary glands (whole mount) from control (21A and 21C) vs. transgenic (21B and 21D) mice overexpressing the hpa gene in all tissues.

FIG. 22 demonstrates binding of bFGF to embryonic fibroblasts. Fibroblasts isolated from 15 days embryos of heparanase transgenic (Tg/Hep) and control mice were incubated with various concentrations of ¹²⁵I-b-FGF. Following incubation cells were washed and the bound b-FGF was quantitated.

FIG. 23 demonstrates heparanase activity in milk of transgenic mice. Milk samples from two independent lines of heparanase transgenic mice, G1 and G3, and from control mice were incubated with 35S labeled ECM for 48 hours. Following incubation degradation products were size fractionated. Heparanase activity is detected in the milk of G3 and G1 transgenic mice and not in control mice.

### DESCRIPTION OF THE PREFERRED EMBODIMENTS

The present invention is of transgenic animals expressing heparanase, which can be used as a model for human disease and for the commercial production of heparanase. The present invention is further of compositions of matter produced by the transgenic animals and of methods of purifying heparanase therefrom. Specifically, the present invention can be used to produce commercial quantities of heparanase and provide non-human mammalian models of metastatic and other diseases.

The principles and operation of the present invention may be better understood with reference to the drawings, examples and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Cloning and Expression of the Human Heparanase Gene (hpa):

The human hpa cDNA, which encodes human heparanase, was cloned from human placenta. It contains an open reading frame, which encodes a polypeptide of 543 amino acids with a calculated molecular weight of 61,192 daltons (2). The cloning procedures of the hpa cDNA and genomic DNA from several species are described in length in U.S. Pat. No. 5,986,822, U.S. patent application Ser. Nos. 09/109,386 and 09/258,892 and PCT Application No. US98/17954, all of which are incorporated herein by reference. An identical cDNA encoding human heparanase was isolated later on from hepatoma cell line SK-hep1 (54). From platelets (55, 57, PCT/US99/01489, PCT/AU98/00898) and from SV40 transformed fibroblasts (56, PCT/EP99/00777).

The genomic locus, which encodes heparanase, spans about 40 kb. It is composed of 12 exons separated by 11 introns and is localized on human chromosome 4.

The ability of the hpa gene product to catalyze degradation of heparan sulfate (HS) in vitro was examined by expressing the entire open reading frame of hpa in High five and Sf21 insect cells, and the mammalian human 293 embryonic kidney cell line expression systems. Extracts of infected or transfected cells were assayed for heparanase catalytic activity. For this purpose, cell lysates were incubated with sulfate labeled, ECM-derived HSPG (peak I), followed by gel filtration analysis (Sepharose 6B) of the reaction mixture. While the substrate alone consisted of high molecular weight material, incubation of the HSPG substrate with lysates of cells infected or transfected with hpa containing vectors resulted in a complete conversion of the high molecular weight substrate into low molecular weight labeled heparan sulfate degradation fragments (see, for example, U.S. patant application Ser. No. 09/071,618, which is incorporated herein by reference.

In other experiments, it was demonstrated that the heparanase enzyme expressed by cells infected with a pFhpa virus is capable of degrading HS complexed to other macromolecular constituents (e.g., fibronectin, laminin, collagen) present in a naturally produced intact ECM (see U.S. patent application Ser. No. 09/109,386, which is incorporated herein by reference), in a manner similar to that reported for highly metastatic tumor cells or activated cells of the immune system (7, 8).

In human primary fibroblasts transfected with the heparanase cDNA the enzyme was localized to the lysosomes.

Preferential Expression of the hpa Gene in Human Breast and Hepatocellular Carcinomas:

Semi-quantitative RT-PCR was employed to evaluate the expression of the hpa gene by human breast carcinoma cell lines exhibiting different degrees of metastasis. A marked increase in hpa gene expression is observed, which correlates to metastatic capacity of non-metastatic MCF-7 breast carcinoma, moderately metastatic MDA 231 and highly metastatic MDA 435 breast carcinoma cell lines. Significantly, the differential pattern of the hpa gene expression correlated with the pattern of heparanase activity.

Expression of the hpa gene in human breast carcinoma was demonstrated by in situ hybridization to archival paraffin embedded human breast tissue. Hybridization of the heparanase antisense riboprobe to invasive duct carcinoma tissue sections resulted in a massive positive staining localized specifically to the carcinoma cells. The hpa gene was also expressed in areas adjacent to the carcinoma showing fibrocystic changes. Normal breast tissue derived from reduction mammoplasty failed to express the hpa transcript. High expression of the hpa gene was also observed in tissue sections derived from human hepatocellular carcinoma specimens but not in normal adult liver tissue. Furthermore, tissue specimens derived from adenocarcinoma of the ovary, squamous cell carcinoma of the cervix and colon adenocarcinoma exhibited strong staining with the hpa RNA probe, as compared to a very low staining of the hpa mRNA in the respective non-malignant control tissues (2).

A preferential expression of heparanase in human tumors versus the corresponding normal tissues was also noted by immunohistochemical staining of paraffin embedded sections with monoclonal anti-heparanase antibodies. Positive cytoplasmic staining was found in neoplastic cells of the colon carcinoma and in dysplastic epithelial cells of a tubulovillous adenoma found in the same specimen while there was little or no staining of the normal looking colon epithelium located away from the carcinoma. Of particular significance was an intense immunostaining of colon adenocarcinoma cells that had metastasized into lymph node, lung and liver, as compared to the surrounding normal tissues (58).

Latent and Active Forms of the Heparanase Protein:

The apparent molecular size of the recombinant enzyme produced in the baculovirus expression system was about 65 kDa. This heparanase polypeptide contains 6 potential N-glycosylation sites. Following deglycosylation by treatment with peptide N-glycosidase, the protein appeared as a 57 kDa band. This molecular weight corresponds to the deduced molecular mass (61,192 daltons) of the 543 amino acid polypeptide encoded by the full length hpa cDNA after cleavage of the predicted 3 kDa signal peptide. No further reduction in the apparent size of the N-deglycosylated protein was observed following concurrent O-glycosidase and neuraminidase treatment. Deglycosylation had no detectable effect on enzymatic activity.

Unlike the baculovirus enzyme, expression of the full length heparanase polypeptide in mammalian cells (e.g., 293 kidney cells, CHO) yielded a major protein of about 50 kDa and a minor of about 65 kDa in cell lysates. Comparison of the enzymatic activity of the two forms, using a semi-quantitative gel filtration assay, revealed that the 50 kDa enzyme is at least 100-200 fold more active than the 65 kDa form. A similar difference was observed when the specific activity of the recombinant 65 kDa baculovirus enzyme was compared to that of the 50 kDa heparanase preparations purified from human platelets, SK-hep-1 cells, or placenta. These results suggest that the 50 kDa protein is a mature processed form of a latent heparanase precursor. Amino terminal sequencing of the platelet heparanase indicated that cleavage occurs between amino acids Gln¹⁵⁷ and Lys¹⁵⁸. As indicated by the hydropathic plot of heparanase, this site is located within a hydrophillic peak, which is likely to be exposed and hence accessible to proteases.

According to Fairbank et al. (57) the precursor is cleaved at three sites to form a heterodimer of a 50 kDa polypeptide (the mature form) that is associated with a 8 kDa peptide.

Although mammalian heparanase can be expressed in vitro in a variety of cell lines of human and non-human origin, there are significant drawbacks to the use of mammalian tissue culture systems for the production of human heparanase in clinically useful quantities such as the expense of growth media, potential contamination with host cell proteins and the limited production capacity of mammalian tissue culture systems.

Thus, there is an important need for an efficient and relatively inexpensive means of producing large quantities of infectious particle-free, human heparanase protein suitable for clinical use and research. The transgenic animal system described below that produces human heparanase recombinantly satisfies this need.

According to one aspect of the present invention there is provided a transgenic non-human animal whose genome comprises an exogenous polynucleotide sequence integrated into the genome, the exogenous polynucleotide sequence including a promoter active in tissues of the non-human, and a region encoding a human heparanase. The promoter and region encoding human heparanase are operably linked such that human heparanase is expressed in at least a portion of the cells of the non-human animal. Depending on the methods of gene transfer, and the integration of the transgene into the host cells, the transgenic non-human animal may be homozygous or heterozygous for the exogenous polynucleotide sequence.

As used herein the term “animal” refers to all multicellular organisms other than human.

As used herein, the term “transgenic” does not encompass classical crossbreeding or in vitro fertilization, but rather denotes animals in which one or more cells receive a recombinant DNA molecule. Although it is highly preferred that this molecule be integrated within the animal's chromosomes, the invention also encompasses the use of extrachromosomally replicating DNA sequences, such as might be engineered into yeast artificial chromosomes.

As used herein the term “transgene” refers to a genetic construct including a polynucleotide encoding a heparanase protein. Preferably, the construct further including an additional polynucleotide harboring at least one cis-acting element which regulates the expression of heparanase from the first polynucleotide. The cis-acting clement(s) are typically located upstream to the coding sequence encoding heparanase. When prepared, such a construct may include additional polynucleotides designed for propagating the construct in bacteria, preferably such additional polynucleotides are removed from the construct prior to the use thereof for generating the transgenic animal.

The phrase “expressing heparanase from a transgene” refers to transcription of heparanase messenger RNA (mRNA) followed by translation thereof into a heparanase. Post translational modifications, including glycosylation, proteolytic cleavage and the like may follow translation.

Heparanase catalytic activity is known to include animal endoglycosidase hydrolyzing activity which is specific for heparin or heparan sulfate proteoglycan substrates, as opposed to the activity of bacterial enzymes (heparinase I, II and III) which degrade heparin or heparan sulfate by means of β-elimination.

Genes encoding mammalian heparanases and the expression and purification thereof are described at length in U.S. Pat. Nos. 5,986,822 and 6,177,545; U.S. patent application Ser. Nos. 09/071,618; 09/109,386; 09/258,892; and PCT applications US/17954, US99/09255 and US99/09256, all of which are incorporated herein by reference. In a preferred embodiment, the gene encoding human heparanase is a polynucleotide encoding a polypeptide having heparanase catalytic activity, the polynucleotide being at least 70%, preferably 80%, more preferably 90% and most preferably 100% homologous to nucleotide coordinates 100 to 1731 of the human hpa heparanase coding sequence (GenBank Accession No. AF144325, to Vlodavsky et al), as determined using default parameters of a DNA sequence analysis software package developed by the Genetic Computer Group (GCG) at the University of Wisconsin. In a more preferred embodiment, the gene encoding human heparanase is a polynucleotide sequence encoding a polypeptide having heparanase catalytic activity, wherein the polypeptide shares at least 70%, preferably 80% more preferably 90% and most preferably 100% homology with human heparanase (GenBank Accession No. AAD41342 to Vlodavsky et al), as determined using default parameters of a DNA sequence analysis software package developed by the Genetic Computer Group (GCG) at the University of Wisconsin.

Further details and references are provided in the Background section above. It will be appreciated by one ordinarily skilled in the art, and it is demonstrated in the above patent documents, that using the human heparanase gene sequence one can readily clone, express and purify recombinant heparanase of any other mammal. This sequence of events, i.e., cloning a gene of one species based on the sequence of the same gene from another species, has proven successful in hundreds of previous cases, especially since the polymerase chain reaction (PCR) may be practiced therefore.

Thus, the term “heparanase” includes polypeptides encoded by a mammalian heparanase gene or a portion thereof, e.g., the portion encoding the mature processed heparanase. The term also includes all of the heparanase species described and discussed in U.S. Pat. No. 6,348,344; and in PCT/US99/09256, both are incorporated herein by reference. These species of heparanase are cleavable into active forms via specific proteases.

The ability to incorporate specific genes into the genome of mammalian embryos has provided a useful in vivo system for the analysis of gene control and expression. The high efficiency transformation of cultured mammalian cells has been accomplished by direct microinjection of specific DNA sequences into the cell nucleus (Capecchi, M., Cell 1980 22:479-488). Gordon, J. W. et al. (Gordon, J. W. et al. Proc. Natl. Acad. Sci. USA 1978 77:7380-7384) demonstrated that DNA could be microinjected into mouse embryos, and found in the resultant offspring. The basic procedure used to produce transgenic mice requires the recovery of fertilized eggs from the oviducts of newly mated female mice. DNA, which contains the gene desired to be transferred into the mouse, is microinjected into the male pronucleus of each fertilized egg. Microinjected eggs are then implanted into the oviducts of one-day pseudopregnant foster mothers and carried to term (Wagner, T. E. et al., Proc. Natl. Acad. Sci. USA 1981 78:6376-6380). Such microinjected genes frequently integrate into chromosomes, are retained throughout development and are transmitted to offspring as Mendelian traits (Wagner, et al, above, and Grosschedl, R. et al. Cell October 1984; 38(3):647-58). Microinjected foreign genes have shown a tendency to be expressed in transgenic mice. Similarly, other mammalian and non-mammalian species (e.g., avian species) are transgenized using similar techniques.

Thus, a variety of transgenic animal species are presently used to produce recombinant proteins.

For mammals, the general approach is to target the expression of the desired protein to the mammary gland using regulatory elements derived from a milk protein gene and then collect and purify the product from milk of animals for the production of the recombinant enzyme. Transgenic cows (see, U.S. Pat. Nos. 6,080,912; 6,013,857), ewes (see, U.S. Pat. Nos. 5,756,687; 6,087,554), goats (see, U.S. Pat. No. 5,843,705) and pigs (U.S. Pat. Nos. 6,030,833; 5,942,435) can be readily engineered to produce recombinant proteins in the milk. Protocols for generating transgenic mammals are provided in, for example, U.S. Pat. Nos. 6,118,045; 6,018,097; 6,015,938; 5,994,616; 5,965,789; 5,965,788; 5,959,171; 5,891,698; 5,880,327; 5,861,313; 5,859,307; 5,850,000; 5,849,997; 5,849,992; 5,831,141; 5,827,690; 5,824,287; 5,759,536; 5,756,687; 5,750,172; 5,716,817; 5,714,345; 5,705,732; 5,700,671; 5,654,182; 5,648,243; 5,639,440; 5,635,355; and 5,602,300, which are incorporated herein by reference.

The following proteins have been successfully expressed in milk: lysosomal proteins; collagen, EC-SOD; bacteriostatic proteins, insulin and many more. While reducing the present invention to practice, recombinant human heparanase protein having native catalytic activity was detected in milk of transgenic female mice expressing the human heparanase gene hpa. Assuming an achievable expression level of 50 mg/L in the milk of a transgenic animal of the invention and a 50% loss of the protein during purification, it can been estimated that about 1 cow (producing 6,000 L of milk yearly), 10 goats, sheep or pigs (producing 500 L of milk yearly), or 5,333 rabbits (producing 0.9 L of milk yearly) could easily supply up to 150 grams of purified human heparanase.

Thus, according to the present invention there is provided a method of producing recombinant human heparanase by obtaining a transgenic non-human mammal having mammary glands, whose genome comprises an exogenous polynucleotide sequence including a promoter active in tissues of the non-human mammal and a region encoding a human heparanase integrated into the genome, the promoter region encoding human heparanase being operably linked in the exogenous polynucleotide such that recombinant human heparanase is secreted into milk produced by the mammary glands, milking the non-human mammal so as to obtain milk containing the recombinant human heparanase, and purifying the recombinant human heparanase from the milk.

Further, according to yet another aspect of the present invention there is provided a composition of matter comprising milk derived from a non-human transgenic mammal, the milk having detectable human heparanase activity. Methods of detecting human heparanase activity include, for example, labeled heparin degradation as described in the Materials and Methods section hereinbelow.

Obtaining milk from a transgenic animal according to the present invention is accomplished by conventional means. See, e.g., McBurney et al., J. Lab. Clin. Med. 64: 485 (1964); Velander et al., Proc Natl. Acad. Sci. USA 89: 12003 (1992), the respective contents of which are incorporated by reference. Heparanase or protein products thereof can be isolated and purified from milk or urine by conventional means without deleteriously affecting activity. A preferred method consists of a combination of anion exchange and immunochromatographies, cryoprecipitations, zinc ion-induced precipitation of either whole milk or milk whey (defatted milk) proteins. For these techniques, see Bringe et al., J. Dairy Res. 56: 543 (1989), the contents of which are incorporated herein by reference.

Importantly, milk is known to contain a number of proteases that have the potential to degrade foreign proteins. These include in the main an alkaline protease with tryptic and chymotryptic activities, a serine protease, a chymotrypsin-like enzyme, an aminopeptidase and an acid protease. As described hereinabove, native heparanase is cleaved by proteolytic enzymes into it's active form. Thus, in one preferred embodiment the transgenic, human heparanase is genetically modified to be cleavable into an active form via a protease. In a most preferred embodiment, the heparanase is processed by an endogenous protease of the animal into an active form.

Alternatively, it may be desirable to protect newly secreted heparanase against proteolytic degradation. Such precautions include rapid processing of the milk after collection and addition to the milk of well known inhibitors of proteolysis, such as are listed in SIGMA CHEMICAL CO. CATALOG (1993 edition) at page 850, the contents of which are incorporated herein by reference. Thus, in a yet further embodiment, the heparanase transgene encodes a processed and active form of heparanase.

In addition, recombinant heparanase may be produced in eggs of transgenic hens. The general approach in this case is to target the expression of the desired protein to the egg-producing cells using regulatory elements derived from an egg protein gene, and then use the egg content as a source of heparanase (e.g., collect and purify the product from eggs of animals for the production of the recombinant enzyme).

Methods for generating transgenic avians, and for production of recombinant proteins secreted into their eggs are provided, for example in U.S. Pat. Nos. 6,080,912; 6,018,097, 5,162,255, 5,854,038. Rapp et al (U.S. patent application Publication No. 20020108132 to Rapp et al.) describe a variety of methods for introduction and expression of trangenes in avian hosts, such as sperm-mediated transfection employing liposomes, direct microinjection of the chick embryos and nuclear transfer. Constructs for secretion of foreign proteins in chicken eggs using chicken lyzozyme gene regulatory sequences (Lampard G R, and Verrinder Gibbins A M, Biochem Cell Biol 2002;80:777-88) and cytomegalovirus promoter (Harvey, A J et al, Nat Biotechnol 2002;20:396-9) have been used successfully for stable expression and direction of biologically active recombinant proteins to the egg white of transgenic chickens. Additionally, chick immunoglobulins are secreted into yolks of developing eggs in large amounts, and their promoters and regulatory sequences can also be useful for expression and transport of foreign proteins in transgenic chicken eggs (see, for example, Morrison S L et al 2002;38:619-625). Using the constructs described hereinabove, human heparanase can be expressed in avian eggs and purified from yolk or egg white.

Thus, according to yet another aspect of the present invention there is provided a method of producing heparanase by obtaining a transgenic female avian having egg producing cells whose genome comprises an exogenous polynucleotide sequence including a promoter active in tissues of the transgenic female avian, and a region encoding a human heparanase integrated into the genome, the promoter and region encoding human heparanase being operably linked such that the recombinant human heparanase is secreted into eggs being produced by egg producing cells, collecting eggs laid by the transgenic female avian so as to obtain eggs containing the human recombinant heparanase, and purifying the recombinant human heparanase from the eggs.

Thus, according to one aspect of the present invention, there is provided a of matter comprising egg yolk and/or white from transgenic avian, the egg yolk and/or white having detectable human heparanase activity.

Methods of purifying heparanase are described in, for example, U.S. Pat. No. 6,348,344 and U.S. patent application Ser. No. 09/071,618, which are incorporated herein by reference.

As is well known in the art, a transgenic animal may include a single locus or several loci harboring the transgene. Southern blot analysis using specific restriction endonucleases can be used to monitor the number of copies of a transgene, so as quantitative PCR. In a specific animal, each such loci may be homozygous or heterozygote. Careful breeding with wild type animals can be used to obtain homozygote or heterozygote animals. In addition, a transgene can be passed from a first genetic background of a first mating strain of a species to another genetic background of a second mating strain of that species by carefully implemented, and well known, breeding protocols. Typically, 3-5 generations are required to do so, depending on the level of heterogeneity between the matting strains.

The expression of the heparanase transgene may be tissue specific, non-specific (all or most tissues), inducible or constitutive. To this end any one of a great repertoire of tissue specific, non-specific, inducible or constitutive promoters can be used. Tissue specific promoters include, but are not limited to, beta-lactoglobulin promoter (Genebank Accession No. X52581), mammary glands (Clark 1998) Rb promoter (Genebank Accession No. M86180), nervous system (Jiang et al. 2000), preproendothelin-1 promoter (Genebank Accession No. U07982), and cardiovascular system (Zaidi et al. 1999). Non tissue-specific constitutive promoters include, but are not limited to, beta-actin promoter and cytomegalovirus promoter. Inducible promoters include, but are not limited to,TetO (tet operator) promoter which is induced by doxycycline and metallothionein promoter (Genebank Accession No. X00504). Metallothionein expression is normally low in most tissues. High expression can be induced by several inflammatory cytokines, protein kinase C activators, and stress agents including heavy metals (Mirault M E et al. Ann N Y Acad Sci Nov. 17, 1994; 738:104-15).

In addition to the abovementioned promoters, using information derived from EST libraries, one can identify tissue specific or non-specific mRNAs and readily clone the promoters responsible for their expression, which reside upstream to the coding sequence in the respective genome. Highly preferred are promoters that are specifically active in mammary gland cells and that involve milk proteins. Among such promoters, highly preferred are the short and long WAP, short and long alpha, beta and kappa casein, alpha-lactalbumin and beta-lactoglobulin (“BLG”) promoters.

Promoters may be selected on the basis of the protein compositions of various milks. For example, the WAP and BLG promoters are particularly useful with transgenic rodents, pigs and sheep. The rodent WAP short and long promoters have been used to express the rat WAP gene, the human tPA gene and the CD4 gene, while the sheep BLG promoter has been used to express the sheep BLG gene, the human alpha-1-antitrypsin gene and the human Factor IX gene. For a review see Clark et al., TIBTECH 5: 20 (1987), the respective content of which is incorporated herein by reference. Preferred among the promoters for carrying out the present invention are the rodent casein and WAP promoters, and the casein, alpha-lactalbumin and BLG promoters from porcine, bovine, equine and ovine (pigs, sheep, goats, cows, horses), rabbits, rodents and domestic pets (dogs and cats). The genes for these promoters have been isolated and their characterizations published. For reviews see Clark et al. (1987), above, and Henninghausen, Protein Expression and Purification4 1: 3 (1990), the respective contents of which are incorporated herein by reference.

DNA sequence information is available for many mammary gland specific genes, in at least one, and often in several organisms. See, e.g., Richards et al., J Biol. Chem. 256, 526-532 (1981) (alpha-lactalbumin rat); Campbell et al., Nucleic Acids Res. 12, 8685-8697 (1984) (rat WAP); Jones et al., J Biol. Chem. 260, 7042-7050 (1985) (rat beta-casein); Yu-Lee & Rosen, J Biol. Chem. 258, 10794-10804 (1983) (rat gamma-casein); Hall, Biochem. J 242, 735-742 (1987) (alpha-lactalbumin human); Stewart, Nucleic Acids Res. 12, 389 (1984) (bovine alpha s1 and kappa casein cDNAs); Gorodetsky et al., Gene 66, 87-96 (1988) (bovine beta casein); Alexander et al., Eur. J Biochem. 178, 395-401 (1988) (bovine kappa casein); Brignon et al., FEBS Lett. 188,48-55 (1977) (bovine alpha S2 casein); Jamieson et al., Gene 61, 85-90 (1987), Ivanov et al., Biol. Chem. Hoppe-Seyler 369, 425-429 (1988), Alexander et al., Nucleic Acids Res. 17, 6739 (1989) (bovine beta lactoglobulin); Vilotte et al., Biochimie 69, 609-620 (1987) (bovine alpha-lactalbumin). The structure and function of the various milk protein genes are reviewed by Mercier & Vilotte, J Dairy Sci. 76, 3079-3098 (1993) (incorporated by reference in its entirety for all purposes). If additional flanking sequence are useful in optimizing expression, such sequences can be cloned using the existing sequences as probes.

Also important to the present invention are regulatory sequences that direct secretion of proteins into milk and/or other body fluids of the transgenic animal. In this regard, both homologous and heterologous regulatory sequences are useful in the invention. Mammary-gland specific regulatory sequences from different organisms can be obtained by screening libraries from such organisms using known cognate nucleotide sequences, or antibodies to cognate proteins as probes. Generally, regulatory sequences known to direct the secretion of milk proteins, such as either signal peptides from milk or the nascent target polypeptide, can be used, although signal sequences can also be used in accordance with this invention that direct the secretion of expressed proteins into other body fluids, particularly blood and urine. Most preferred for the transgenic mouse are the regulatory sequences for the WAP protein.

Tissue specific or constitutive expression can be used according to the present invention not only to produce commercial quantities of heparanase, as described above and exemplified in the examples section that follows, but also to generate animal models for a variety of human diseases and for other applications as is further delineated hereinafter. Methods for the generation and use of transgenic mouse models of human disease are described in detail in, for example, U.S. Pat. Nos. 6,509,515; 6,512,161; 6,515,197; 6,521,815, and in references described hereinabove.

Any one or more of several methods can be used to monitor the expression of a transgene. These include tissue specific Northern blot; tissue specific RT-PCR; in situ hybridization; immunohistochemistry; and protein activity assays. These methods are well known in the art and are described in detail in, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989).

Transgenic mice expressing human heparanase of the present invention can be crossed with other mice strains with defined susceptibility for disease (e.g., mammary cancer, Guy et al. Proc. Natl. Acad. Sci. USA 1992, 89:10578-82, prostate cancer, Tomoyuki Shirai et al. Mutation Research 2000, 412:219-226). Since heparanase expression has been implicated in progression of breast cancer, for example, transgenic mice expressing human heparanse could be crossed with breast cancer-related mouse models, such as Igf1 and TGFA transgenic strains (available from Jackson Labs, Maine USA), under control of an inducible promoter, for investigation of interaction between the transgene products. Efficacy of anti-cancer drugs and therapies can also be tested in such a model with greater accuracy than in existing in vitro or in vivo models. Similarly, induction of inflammation and autoimmune disorders in heparanase overexpressing mice will shed light on heparanase involvement in such conditions. The effect of heparanase expression on development of which involve heparan sulfate and HS bound growth factors can also be evaluated and may suggest possible uses for therapy using gene therapy or the recombinant enzyme. Such conditions, which can be induced in the transgenic animals include tissue repair (e.g., wound healing, bone repair and nerve regeneration) where heparanase is suggested to increase the availability of HS bound growth factors and facilitate cell proliferation and migration, as well as pathological processes, which develop as a result of insufficient blood supply (e.g., cerebral, cardiac and diabetic ulcer ischemia), where heparanase is suggested to induce neovascularization. Transgenic mice can also serve as a model for studying the effect of heparanase on bone metabolism, including osteoporosis, either age related or in response to ovariohysterectomy, glucocorticoid therapy and heparin therapy and on amyloidosis, such as Alzheimer disease or renal.

Constitutive overexpression of heparanase may provide essential information regarding life long effects such as chronic toxicity as reflected by life span and aging, and the effect of heparanase on fertility and reproduction considering the suggested role of heparanase in embryo implantation (63).

As described in detail hereinabove, heparanase activity is crucial for the integrity of the ECM, and has been implicated in tumorigenesis, inflammation, malignancy, viral infection, tumor angiogenesis, atherogenesis and metastasis. Thus, for example, transgenic mice overexpressing heparanase provides a powerful tool for studying the role of heparanase, metabolism of heparan sulfate and HS bound proteins in normal and pathological processes. The transgene expression pattern may reflect a specific mode of protein administration. In animals which express the transgene constitutively in all tissues, heparanase is provided chronically and systemically.

The present invention offers several advantages over existing models for metastasis. Transgenic mice expressing high levels of human heparanase can be exposed to known carcinogens and cancer risk factors, and potential for metastatic development of cancerous cells observed in these animals. Metastatic changes provide a particular advantage in screening protocols for agents that can be used in treatment for cancerous disease such as colorectal cancer and melanoma. Furthermore, manipulation of expression of transgene expression is well known in the art (see abovementioned US patents). Organ-specific regulatory sequences, specifically promoters, can be used to target overexpression of the human heparanase transgene to tissues of interest. Similarly, integration of the transgene into the Y chromosome can provide sex-specific expression (see, for example, Neilsen et al Canc Res 1992;52:3733-38).

In addition transgenic animals provide a source for primary cells overexpressing heparanase, such as embryonic cells, bone marrow cells, bone marrow stromal cells, spermatogonia, keratinocytes and sex cells (spermatocytes and oocytes). Such cells can be isolated using protocols for cell isolation and/or enrichment which are well known in the art. Based on the observation described in the Background section above that heparanase increases cell extravasation, such cells can be transplanted for immunotherapy, cell and gene therapy. Similarly, transgenic organs can be used for xenotransplantation, skin and embryo implantation, whereas sex cells can be used for in vitro fertilization (oocytes) and artificial insemination (spermatocytes).

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Cloning and Expressing the Heparanase Gene

### Materials and Experimental Methods

Purification and characterization of heparanase from a human hepatoma cell line and human placenta: A human hepatoma cell line (Sk-hep-1) was chosen as a source for purification of a human tumor-derived heparanase. Purification was essentially as described in U.S. Pat. No. 5,362,641 to Fuks, which is incorporated by reference as if fully set forth herein. Briefly, 500 liter, 5×10¹¹ cells were grown in suspension and the heparanase enzyme was purified about 240,000 fold by applying the following steps: (i) cation exchange (CM-Sephadex) chromatography performed at pH 6.0, 0.3-1.4 M NaCl gradient; (ii) cation exchange (CM-Sephadex) chromatography performed at pH 7.4 in the presence of 0.1% CHAPS, 0.3-1.1 M NaCl gradient; (iii) heparin-Sepharose chromatography performed at pH 7.4 in the presence of 0.1% CHAPS, 0.35-1.1 M NaCl gradient; (iv) ConA-Sepharose chromatography performed at pH 6.0 in buffer containing 0.1% CHAPS and 1 M NaCl, elution with 0.25 M α-methyl mannoside; and (v) HPLC cation exchange (Mono-S) chromatography performed at pH 7.4 in the presence of 0.1% CHAPS, 0.25-1 M NaCl gradient.

Active fractions were pooled, precipitated with TCA and the precipitate subjected to SDS polyacrylamide gel electrophoresis and/or tryptic digestion and reverse phase HPLC. Tryptic peptides of the purified protein were separated by reverse phase HPLC (C8 column) and homogeneous peaks were subjected to amino acid sequence analysis.

The purified enzyme was applied to reverse phase HPLC and subjected to N-terminal amino acid sequencing using the amino acid sequencer (Applied Biosystems).

Cells: Cultures of bovine corneal endothelial cells (BCECs) were established from steer eyes as previously described (31, 72). Stock cultures were maintained in DMEM (1 g glucose/liter) supplemented with 10% newborn calf serum and 5% FCS. bFGF (1 ng/ml) was added every other day during the phase of active cell growth (73, 74).

Preparation of dishes coated with ECM: BCECs (second to fifth passage) were plated into 4-well plates at an initial density of 2×10⁵ cells/ml, and cultured in sulfate-free Fisher medium plus 5% dextran T-40 for 12 days. Na₂³⁵SO₄ (25 μCi/ml) was added on day 1 and 5 after seeding and the cultures were incubated with the label without medium change. The subendothelial ECM was exposed by dissolving (5 min., room temperature) the cell layer with PBS containing 0.5% Triton X-100 and 20 mM NH₄OH, followed by four washes with PBS. The ECM remained intact, free of cellular debris and firmly attached to the entire area of the tissue culture dish (31, 34).

To prepare soluble sulfate labeled proteoglycans (peak I material), the ECM was digested with trypsin (25 μg/ml, 6 h, 37° C.), the digest was concentrated by reverse dialysis and the concentrated material was applied onto a Sepharose 6B gel filtration column. The resulting high molecular weight material (Kav<0.2, peak I) was collected. More than 80% of the labeled material was shown to be composed of heparan sulfate proteoglycans (26, 75).

Heparanase activity: Cells (1×10⁶/35-mm dish), cell lysates or conditioned media were incubated on top of ³⁵S-labeled ECM (18 h, 37° C.) in the presence of 20 mM phosphate buffer (pH 6.2). Cell lysates and conditioned media were also incubated with sulfate labeled peak I material (10-20 μl). The incubation medium was collected, centrifuged (18,000×g, 4° C., 3 min.), and sulfate labeled material analyzed by gel filtration on a Sepharose CL-6B column (0.9×30 cm). Fractions (0.2 ml) were eluted with PBS at a flow rate of 5 ml/h and counted for radioactivity using Bio-fluor scintillation fluid. The excluded volume (Vₒ) was marked by blue dextran and the total included volume (Vₜ) by phenol red. The latter was shown to comigrate with free sulfate (76, 26, 35). Degradation fragments of HS side chains were eluted from Sepharose 6B at 0.5<Kav<0.8 (peak II) (76, 26,35). A nearly intact HSPG released from ECM by trypsin—and, to a lower extent, during incubation with PBS alone—was eluted next to Vₒ (Kav<0.2, peak I). Recoveries of labeled material applied on the columns ranged from 85 to 95% in different experiments (26). Each experiment was performed at least three times and the variation of elution positions (Kav values) did not exceed +/−15%.

Cloning of hpa cDNA: cDNA clones 257548 and 260138 were obtained from the I.M.A.G.E Consortium (2130 Memorial Parkway SW, Hunstville, Ala. 35801). The cDNAs were originally cloned in EcoRI and NotI cloning sites in the plasmid vector pT3T7D-Pac. Although these clones are reported to be somewhat different, DNA sequencing demonstrated that these clones are identical to one another. Marathon RACE (rapid amplification of cDNA ends) human placenta (poly-A) cDNA composite was a gift of Prof. Yossi Shiloh of Tel Aviv University. This composite is vector free, as it includes reverse transcribed cDNA fragments to which double, partially single stranded adapters are attached on both sides. The construction of the specific composite employed is described in reference 77.

Amplification of hp3 PCR fragment was performed according to the protocol provided by Clontech laboratories. The template used for amplification was a sample taken from the above composite. The primers used for amplification were:

First step: 5′-primer: AP1: 5′-CCATCCTAATACGACTCACT ATAGGGC-3′, SEQ ID NO:1; 3′-primer: HPL229: 5′-GTAGTGATGCCA TGTAACTGAATC-3′, SEQ ID NO:2.

Second step: nested 5′-primer: AP2: 5′-ACTCACTATAGGGCTCG AGCGGC-3′, SEQ ID NO:3; nested 3′-primer: HPL171: 5′-GCATCTTAGCCGTCTTTCTTCG-3′, SEQ ID NO:4. The HPL229 and HPL171 were selected according to the sequence of the EST clones. They include nucleotides 933-956 and 876-897 of SEQ ID NO:9, respectively.

PCR program was 94° C.-4 min., followed by 30 cycles of 94° C.-40 sec., 62° C.-1 min., 72° C.-2.5 min. Amplification was performed with Expand High Fidelity (Boehringer Mannheim). The resulting ca. 900 bp hp3 PCR product was digested with BfrI and PvuII. Clone 257548 (phpa1) was digested with EcoRI, followed by end filling and was then further digested with BfrI. Thereafter the PvuII-BfrI fragment of the hp3 PCR product was cloned into the blunt end—BfrI end of clone phpal which resulted in having the entire cDNA cloned in pT3T7-pac vector, designated phpa2.

RT-PCR: RNA was prepared using TRI-Reagent (Molecular research center Inc.) according to the manufacturer instructions. 1.25 μg were taken for reverse transcription reaction using MuMLV Reverse transcriptase (Gibco BRL) and Oligo (dT)₁₅ primer, SEQ ID NO:5, (Promega). Amplification of the resultant first strand cDNA was performed with Taq polymerase (Promega). The following primers were used:

HPU-355: 5′-TTCGATCCCAAGAAGGAATCAAC-3′, SEQ ID NO:6, nucleotides 372-394 in SEQ ID NOs:9 or 11.

HPL-229: 5′-GTAGTGATGCCATGTAACTGAATC-3′, SEQ ID NO:7, nucleotides 933-956 in SEQ ID NOs:9 or 11.

PCR program: 94° C.-4 min., followed by 30 cycles of 94° C.-40 sec., 62° C.-1 min., 72° C.-1 min.

Alternatively, total RNA was prepared from cell cultures using Tri-reagent (Molecular Research Center, Inc.) according to the manufacturer recommendation. Poly A+ RNA was isolated from total RNA using mRNA separator (Clontech). Reverse transcription was performed with total RNA using Superscript II (GibcoBRL). PCR was performed with Expand high fidelity (Bochringer Mannheim). Primers used for amplification were as follows:

SEQ ID NO:24

Hpu-685, 5′-GAGCAGCCAGGTGAGCCCAAGAT-3′,

SEQ ID NO:25

Hpu-355, 5′-TTCGATCCCAAGAAGGAATCAAC-3′,

SEQ ID NO:26

Hpu 565, 5′-AGCTCTGTAGATGTGCTATACAC-3′,

SEQ ID NO:27

Hpl 967, 5′-TCAGATGCAAGCAGCAACTTTGGC-3′,

SEQ ID NO:28

Hpl 171, 5′-GCATCTTAGCCGTCTTTCTTCG-3′,

SEQ ID NO:29

Hpl 229, 5′-GTAGTGATGCCATGTAACTGAATC-3′,

PCR reaction was performed as follows: 94° C. 3 minutes, followed by 32 cycles of 94° C. 40 seconds, 64° C. 1 minute, 72° C. 3 minutes, and one cycle 72° C., 7 minutes.

Expression of recombinant heparanase in insect cells: Cells, High Five and Sf21 insect cell lines were maintained as monolayer cultures in SF900II-SFM medium (GibcoBRL).

Recombinant Baculovirus: Recombinant virus containing the hpa gene was constructed using the Bac to Bac system (GibcoBRL). The transfer vector pFastBac was digested with SalI and NotI and ligated with a 1.7 kb fragment of phpa2 digested with XhoI and NotI. The resulting plasmid was designated pFasthpa2. An identical plasmid designated pFasthpa4 was prepared as a duplicate and both independently served for further experimentations. Recombinant bacmid was generated according to the instructions of the manufacturer with pFasthpa2, pFasthpa4 and with pFastBac. The latter served as a negative control. Recombinant bacmid DNAs were transfected into Sf21 insect cells. Five days after transfection recombinant viruses were harvested and used to infect High Five insect cells, 3×10⁶ cells in T-25 flasks. Cells were harvested 2-3 days after infection. 4×10⁶ cells were centrifuged and resuspended in a reaction buffer containing 20 mM phosphate citrate buffer, 50 mM NaCl. Cells underwent three cycles of freeze and thaw and lysates were stored at −80° C. Conditioned medium was stored at 4° C.

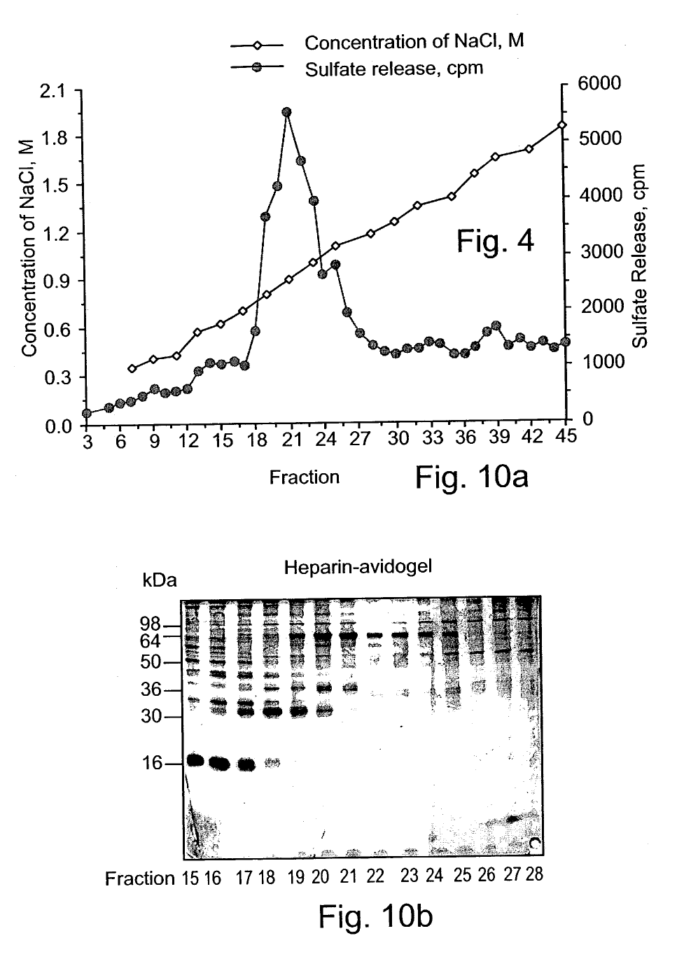

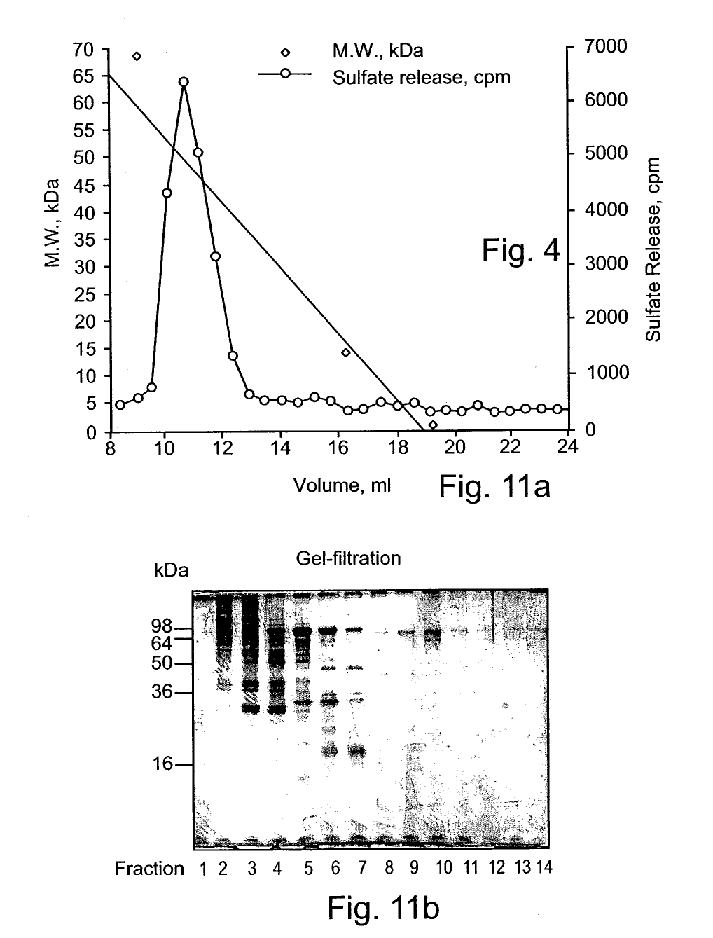

Partial purification of recombinant heparanase: Partial purification of recombinant heparanase was performed by heparin-Sepharose column chromatography followed by Superdex 75 column gel filtration. Culture medium (150 ml) of Sf21 cells infected with pFhpa4 virus was subjected to heparin-Sepharose chromatography. Elution of 1 ml fractions was performed with 0.35-2 M NaCl gradient in presence of 0.1% CHAPS and 1 mM DTT in 10 mM sodium acetate buffer, pH 5.0. A 25 μl sample of each fraction was tested for heparanase activity. Heparanase activity was eluted at the range of 0.65-1.1 M NaCl (fractions 18-26, FIG. 10a). 5 μl of each fraction was subjected to 15% SDS-polyacrylamide gel electrophoresis followed by silver nitrate staining. Active fractions eluted from heparin-Sepharose (FIG. 10a) were pooled and concentrated (×6) on YM3 cut-off membrane. 0.5 ml of the concentrated material was applied onto 30 ml Superdex 75 FPLC column equilibrated with 10 mM sodium acetate buffer, pH 5.0, containing 0.8 M NaCl, 1 mM DTT and 0.1% CHAPS. Fractions (0.56 ml) were collected at a flow rate of 0.75 ml/min. Aliquots of each fraction were tested for heparanase activity and were subjected to SDS-polyacrylamide gel electrophoresis followed by silver nitrate staining (FIG. 11b).

PCR amplification of genomic DNA: 94° C. 3 minutes, followed by 32 cycles of 94° C. 45 seconds, 64° C. 1 minute, 68° C. 5 minutes, and one cycle at 72° C., 7 minutes. Primers used for amplification of genomic DNA included:

GHpu-L3 5′-AGGCACCCTAGAGATGTTCCAG-3′, SEQ ID NO:30

GHpl-L6 5′-GAAGATTTCTGTTTCCATGACGTG-3′, SEQ ID NO:31.

Screening of genomic libraries: A human genomic library in Lambda phage EMBLE3 SP6/T7 (Clontech, Paulo Alto, Calif.) was screened. 5×10⁵ plaques were plated at 5×10⁴ pfu/plate on NZCYM agar/top agarose plates. Phages were absorbed on nylon membranes in duplicates (Qiagen). Hybridization was performed at 65° C. in 5×SSC, 5× Denhart's, 10% dextran sulfate, 100 μg/ml Salmon sperm, ³²p labeled probe (10⁶ cpm/ml). A 1.6 kb fragment, containing the entire hpa cDNA was labeled by random priming (Boehringer Mannheim). Following hybridization membranes were washed once with 2×SSC, 0.1% SDS at 65° C. for 20 minutes, and twice with 0.2×SSC, 0.1% SDS at 65° C. for 15 minutes. Hybridizing plaques were picked, and plated at 100 pfu/plate. Hybridization was performed as above and single isolated positive plaques were picked.

Phage DNA was extracted using a Lambda DNA extraction kit (Qiagen). DNA was digested with XhoI and EcoRI, separated on 0.7% agarose gel and transferred to nylon membrane Hybond N+ (Amersham). Hybridization and washes were performed as above.

cDNA Sequence analysis: Sequence determinations were performed with vector specific and gene specific primers, using an automated DNA sequencer (Applied Biosystems, model 373A). Each nucleotide was read from at least two independent primers.

Genomic sequence analysis: Large-scale sequencing was performed by Commonwealth Biotechnology Incorporation.

Isolation of mouse hpa: Mouse hpa cDNA was amplified from either Marathon ready cDNA library of mouse embryo or from mRNA isolated from mouse melanoma cell line BL6, using the Marathon RACE kit from Clontech. Both procedures were performed according to the manufacturer's recommendation.

Primers used for PCR amplification of mouse hpa:

SEQ ID NO:32

Mhpl773 5′-CCACACTGAATGTAATACTGAAGTG-3′,

SEQ ID NO:33

MHp1736 5′-CGAAGCTCTGGAACTCGGCAAG-3′,

SEQ ID NO:34

MHpI83 5′-GCCAGCTGCAAAGGTGTTGGAC-3′,

SEQ ID NO:35

Mhpll52 5′-AACACCTGCCTCATCACGACTTC-3′,

SEQ ID NO:36

Mhpll 14 5′-GCCAGGCTGGCGTCGATGGTGA-3′,

SEQ ID NO:37

MHpI1O3 5′-GTCGATGGTGATGGACAGGAAC-3′,

SEQ ID NO:38 -

Apl 5 ′-GTAA TA CGA CTCA CTA TA GGGC-3′,
(Genome walker)

SEQ ID NO:39 -

Ap2 5′-ACTATAGGGCACGCGTGGT-3′,
(Genome walker)

SEQ ID NO:40 -

Apl 5′-CCATCCTAATACGACTCACTATAGGGC-3′,
(Marathon RACE)

SEQ ID NO:41 -

Ap2 5′-ACTCACTATAGGGCTCGAGCGGC-3′,
(Marathon RACE)

Southern analysis of genomic DNA: Genomic DNA was extracted from animal or from human blood using Blood and cell culture DNA maxi kit (Qiagene). DNA was digested with EcoRI, separated by gel electrophoresis and transferred to a nylon membrane Hybond N+ (Amersham). Hybridization was performed at 68° C. in 6×SSC, 1% SDS, 5× Denharts, 10% dextran sulfate, 100 μg/ml salmon sperm DNA, and ³²p labeled probe. A 1.6 kb fragment, containing the entire hpa cDNA was used as a probe. Following hybridization, the membrane was washed with 3×SSC, 0.1% SDS, at 68° C. and exposed to X-ray film for 3 days. Membranes were then washed with 1×SSC, 0.1% SDS, at 68° C. and were reexposed for 5 days.

Construction of hpa promoter-GFP expression vector: Lambda DNA of phage L3, was digested with SacI and BglII, resulting in a 1712 bp fragment which contained the hpa promoter (877-2688 of SEQ ID NO:42). The pEGFP-1 plasmid (Clontech) was digested with BglII and SacI and ligated with the 1712 bp fragment of the hpa promoter sequence. The resulting plasmid was designated phpEGL. A second hpa promoter-GFP plasmid was constructed containing a shorter fragment of the hpa promoter region: phpEGL was digested with HindIII, and the resulting 1095 bp fragment (nucleotides 1593-2688 of SEQ ID NO:42) was ligated with HindIII digested pEGFP-1. The resulting plasmid was designated phpEGS.

Computer analysis of sequences: Homology searches were performed using several computer servers, and various databases. Blast 2.0 service, at the NCBI server was used to screen the protein database swplus and DNA databases such as GenBank, EMBL, and the EST databases. Blast 2.0 search was performed using the basic search option of the NCBI server. Sequence analysis and alignments were done using the DNA sequence analysis software package developed by the Genetic Computer Group (GCG) at the university of Wisconsin. Alignments of two sequences were performed using Bestfit (gap creation penalty—12, gap extension penalty—4). Protein homology search was performed with the Smith-Waterman algorithm, using the Bioaccelerator platform developed by Compugene. The protein database swplus was searched using the following parameters: gapop: 10.0, gapext: 0.5, matrix: blosum62. Blocks homology was performed using the Blocks WWW server developed at Fred Hutchinson Cancer Research Center in Seattle, Wash., USA. Secondary structure prediction was performed using the PHD server—Profile network Prediction Heidelberg. Fold recognition (threading) was performed using the UCLA-DOE structure prediction server. The method used for prediction was gonnet+predss. Alignment of three sequences was performed using the pileup application (gap creation penalty—5, gap extension penalty—1). Promoter analysis was performed using TSSW and TSSG programs (BCM Search Launcher Human Genome Center, Baylor College of Medicine, Houston Tex.).

### Example 1

### Cloning of Human hpa cDNA

Purified fraction of heparanase isolated from human hepatoma cells (SK-hep-1) was subjected to tryptic digestion and microsequencing. EST (Expressed Sequence Tag) databases were screened for homology to the back translated DNA sequences corresponding to the obtained peptides. Two EST sequences (accession Nos. N41349 and N45367) contained a DNA sequence encoding the peptide YGPDVGQPR (SEQ ID NO:8). These two sequences were derived from clones 257548 and 260138 (I.M.A.G.E Consortium) prepared from 8 to 9 weeks placenta cDNA library (Soares). Both clones which were found to be identical contained an insert of 1020 bp which included an open reading frame (ORF) of 973 bp followed by a 3′ untranslated region of 27 bp and a Poly A tail. No translation start site (AUG) was identified at the 5′ end of these clones.

Cloning of the missing 5′ end was performed by PCR amplification of DNA from a placenta Marathon RACE cDNA composite. A 900 bp fragment (designated hp3), partially overlapping with the identified 3′ encoding EST clones was obtained.

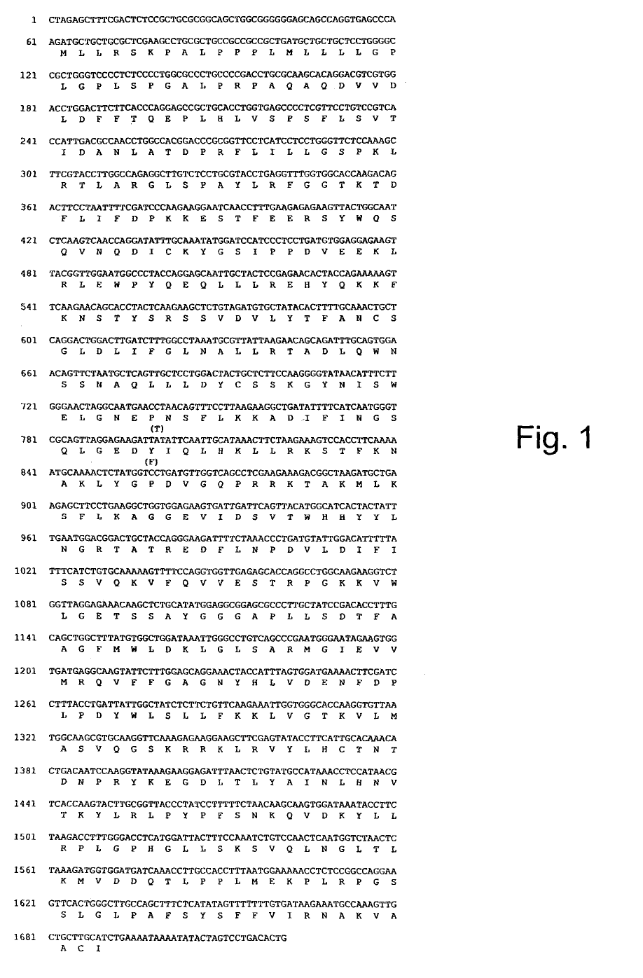

The joined cDNA fragment, 1721 bp long (SEQ ID NO:9), contained an open reading frame which encodes, as shown in FIG. 1 and SEQ ID NO:11, a polypeptide of 543 amino acids (SEQ ID NO:10) with a calculated molecular weight of 61,192 daltons. The 3′ end of the partial cDNA inserts contained in clones 257548 and 260138 started at nucleotide G⁷²¹ of SEQ ID NO:9 and FIG. 1.

As further shown in FIG. 1, there was a single sequence discrepancy between the EST clones and the PCR amplified sequence, which led to an amino acid substitution from Tyr²⁴⁶ in the EST to Phe²⁴⁶ in the amplified cDNA. The nucleotide sequence of the PCR amplified cDNA fragment was verified from two independent amplification products. The new gene was designated hpa.

As stated above, the 3′ end of the partial cDNA inserts contained in EST clones 257548 and 260138 started at nucleotide 721 of hpa (SEQ ID NO:9). The ability of the hpa cDNA to form stable secondary structures, such as stem and loop structures involving nucleotide stretches in the vicinity of position 721 was investigated using computer modeling. It was found that stable stem and loop structures are likely to be formed involving nucleotides 698-724 (SEQ ID NO:9). In addition, a high GC content, up to 70%, characterizes the 5′ end region of the hpa gene, as compared to about only 40% in the 3′ region. These findings may explain the immature termination and therefore lack of 5′ ends in the EST clones.

To examine the ability of the hpa gene product to catalyze degradation of heparan sulfate in an in vitro assay the entire open reading frame was expressed in insect cells, using the Baculovirus expression system. Extracts of cells, infected with virus containing the hpa gene, demonstrated a high level of heparan sulfate degradation activity, while cells infected with a similar construct containing no hpa gene had no such activity, nor did non-infected cells. These results are further demonstrated in the following Examples.

### Example 2

### Degradation of Soluble ECM-Derived HSPG

Monolayer cultures of High Five cells were infected (72 h, 28° C.) with recombinant Bacoluvirus containing the pFasthpa plasmid or with control virus containing an insert free plasmid. The cells were harvested and lysed in heparanase reaction buffer by three cycles of freezing and thawing. The cell lysates were then incubated (18 h, 37° C.) with sulfate labeled, ECM-derived HSPG (peak I), followed by gel filtration analysis (Sepharose 6B) of the reaction mixture.

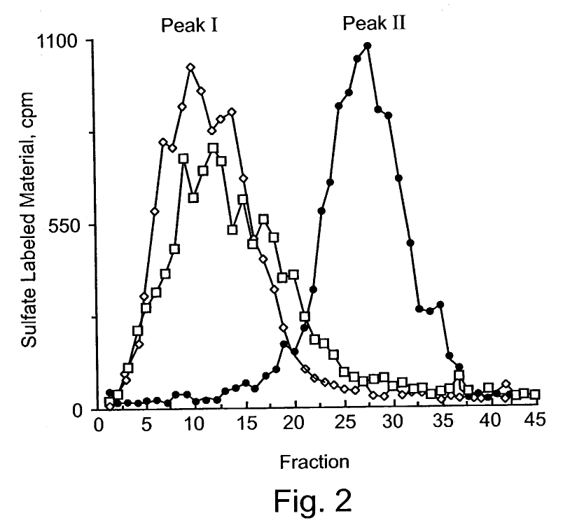

As shown in FIG. 2, the substrate alone included almost entirely high molecular weight (Mr) material eluted next to Vₒ (peak I, fractions 5-20, Kav<0.35). A similar elution pattern was obtained when the HSPG substrate was incubated with lysates of cells that were infected with control virus. In contrast, incubation of the HSPG substrate with lysates of cells infected with the hpa containing virus resulted in a complete conversion of the high Mr substrate into low Mr labeled degradation fragments (peak II, fractions 22-35, 0.5<Kav<0.75).

Fragments eluted in peak II were shown to be degradation products of heparan sulfate, as they were (i) 5- to 6-fold smaller than intact heparan sulfate side chains (Kav approx. 0.33) released from ECM by treatment with either alkaline borohydride or papain; and (ii) resistant to further digestion with papain or chondroitinase ABC, and susceptible to deamination by nitrous acid (10, 26).

Similar results (not shown) were obtained with Sf21 cells. Again, heparanase activity was detected in cells infected with the hpa containing virus (pFhpa), but not with control virus (pF). This result was obtained with two independently generated recombinant viruses. Lysates of control not infected High Five cells failed to degrade the HSPG substrate.

In subsequent experiments, the labeled HSPG substrate was incubated with medium conditioned by infected High Five or Sf21 cells.

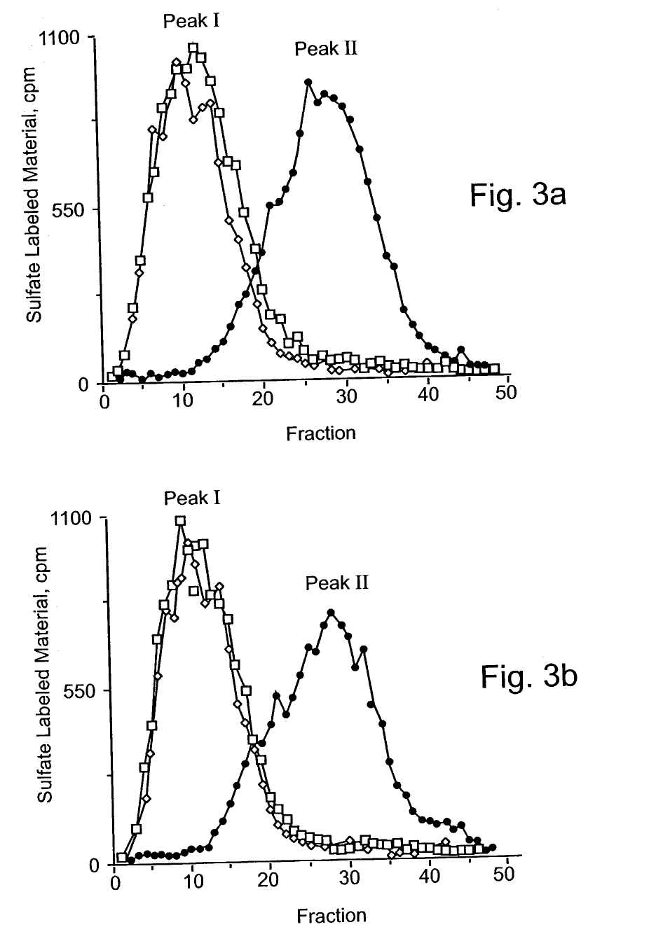

As shown in FIGS. 3a-b, heparanase activity, reflected by the conversion of the high Mr peak I substrate into the low Mr peak II which represents HS degradation fragments, was found in the culture medium of cells infected with the pFhpa2 or pFhpa4 viruses, but not with the control pF1 or pF2 viruses. No heparanase activity was detected in the culture medium of control non-infected High Five or Sf21 cells.

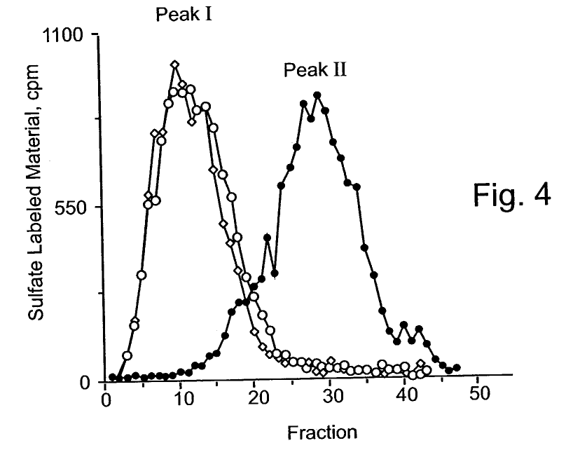

The medium of cells infected with the pFhpa4 virus was passed through a 50 kDa cut off membrane to obtain a crude estimation of the molecular weight of the recombinant heparanase enzyme. As demonstrated in FIG. 4, all the enzymatic activity was retained in the upper compartment and there was no activity in the flow through (<50 kDa) material. This result is consistent with the expected molecular weight of the hpa gene product.

In order to further characterize the hpa product the inhibitory effect of heparin, a potent inhibitor of heparanase mediated HS degradation (77) was examined.

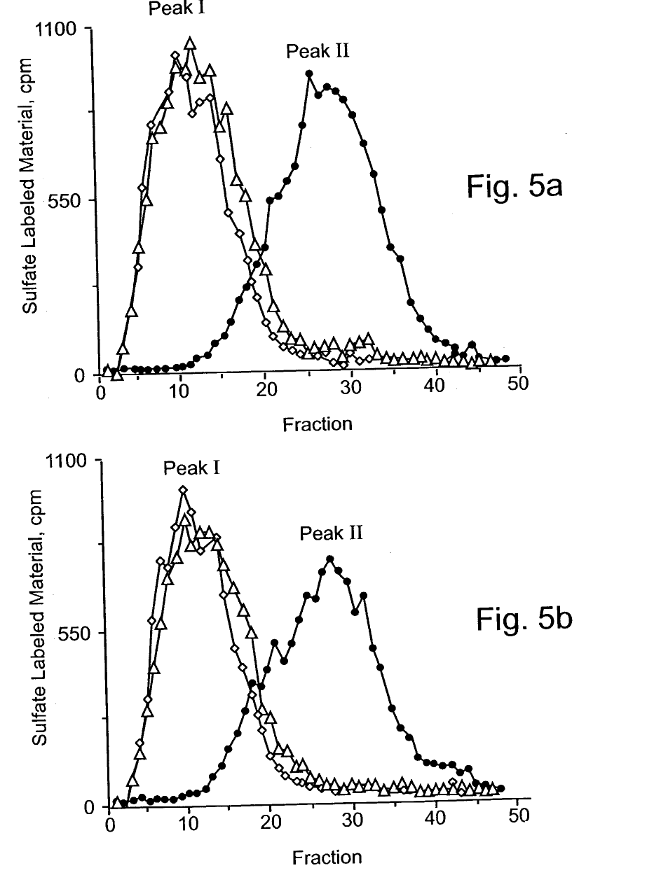

As demonstrated in FIGS. 5a-b, conversion of the peak I substrate into peak II HS degradation fragments was completely abolished in the presence of heparin.

Altogether, these results indicate that the heparanase enzyme is expressed in an active form by insect cells infected with Baculovirus containing the newly identified human hpa gene.

### Example 3

### Degradation of HSPG in Intact ECM

Next, the ability of intact infected insect cells to degrade HS in intact, naturally produced ECM was investigated. For this purpose, High Five or Sf21 cells were seeded on metabolically sulfate labeled ECM followed by infection (48 h, 28° C.) with either the pFhpa4 or control pF2 viruses. The pH of the medium was then adjusted to pH 6.2-6.4 and the cells further incubated with the labeled ECM for another 48 h at 28° C. or 24 h at 37° C. Sulfate labeled material released into the incubation medium was analyzed by gel filtration on Sepharose 6B.

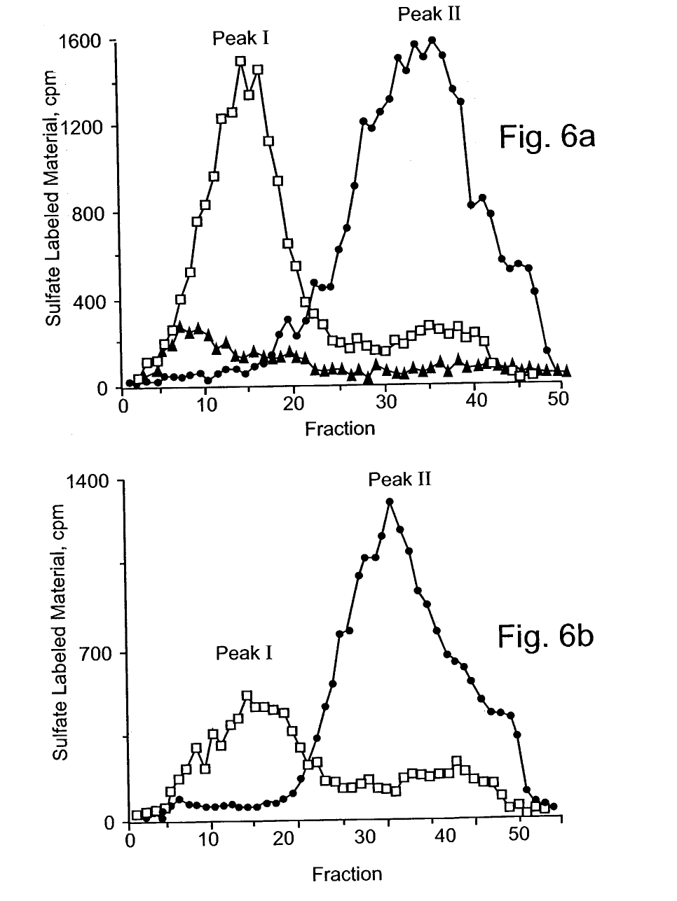

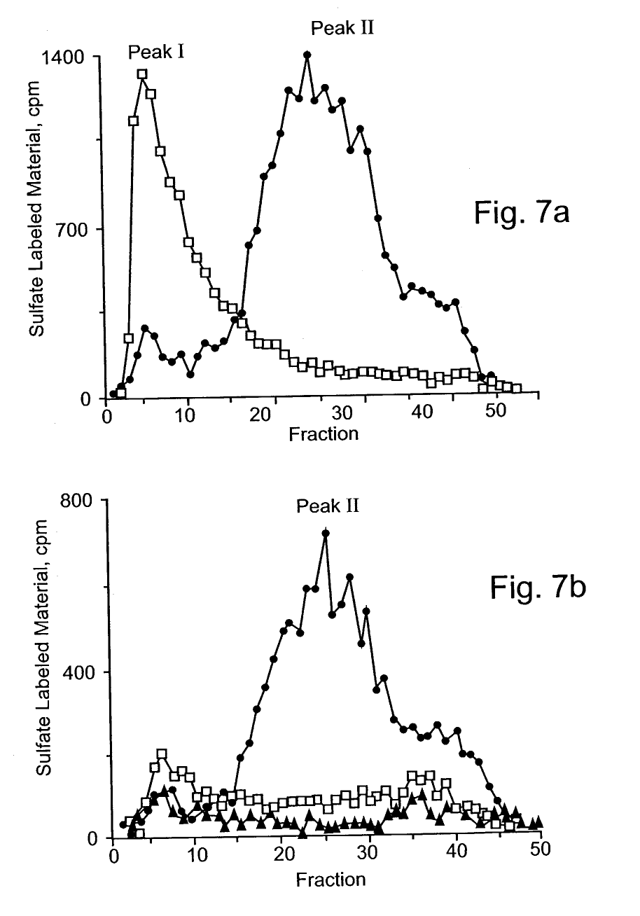

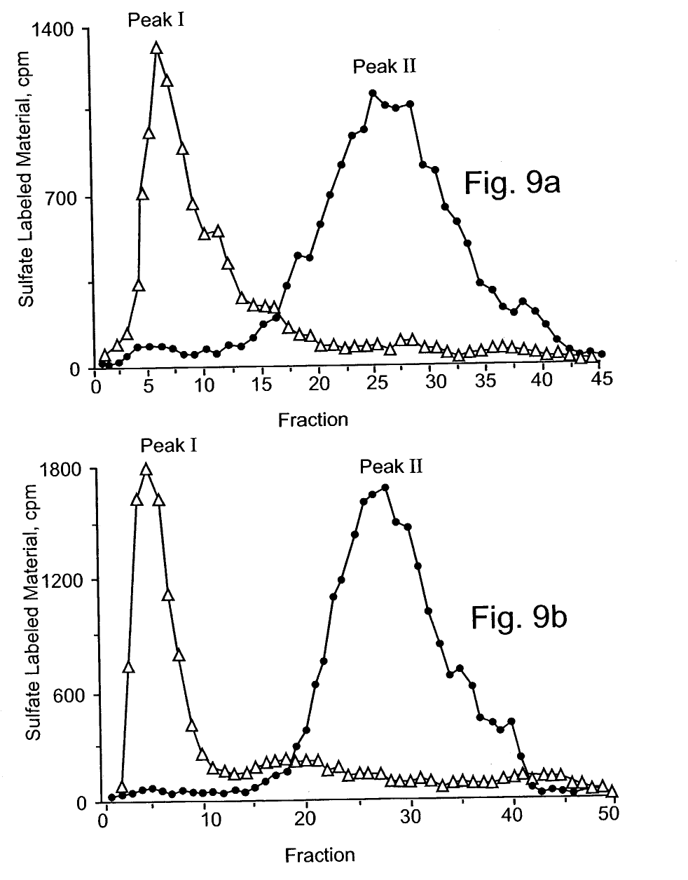

As shown in FIGS. 6a-b and 7a-b, incubation of the ECM with cells infected with the control pF2 virus resulted in a constant release of labeled material that consisted almost entirely (>90%) of high Mr fragments (peak I) eluted with or next to Vₒ. It was previously shown that a proteolytic activity residing in the ECM itself and/or expressed by cells is responsible for release of the high Mr material (10). This nearly intact HSPG provides a soluble substrate for subsequent degradation by heparanase, as also indicated by the relatively large amount of peak I material accumulating when the heparanase enzyme is inhibited by heparin (10, 76, 78, FIG. 9). On the other hand, incubation of the labeled ECM with cells infected with the pFhpa4 virus resulted in release of 60-70% of the ECM-associated radioactivity in the form of low Mr sulfate-labeled fragments (peak II, 0.5<Kav<0.75), regardless of whether the infected cells were incubated with the ECM at 28° C. or 37° C. Control intact non-infected Sf21 or High Five cells failed to degrade the ECM HS side chains.

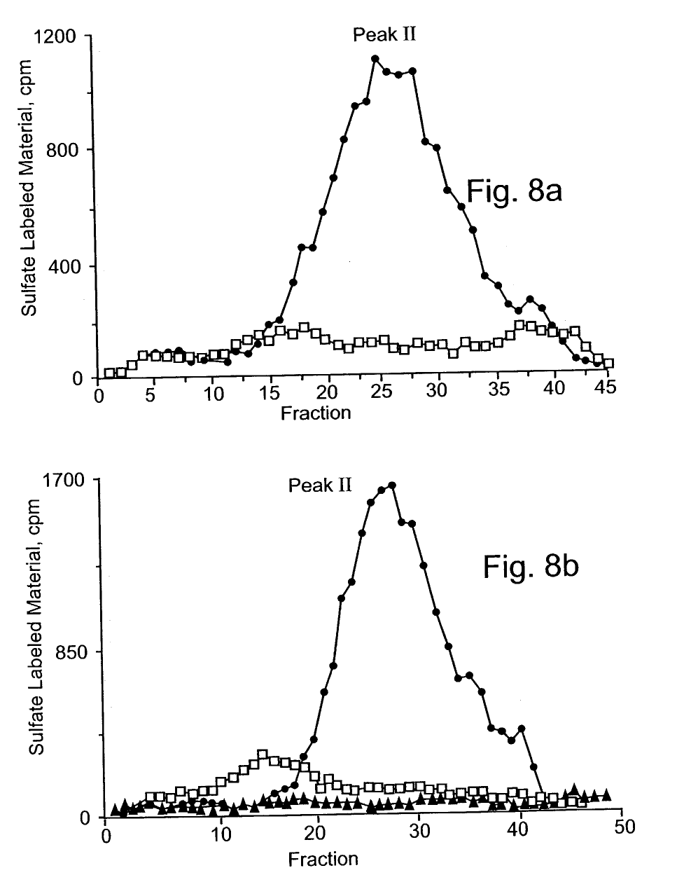

In subsequent experiments, as demonstrated in FIGS. 8a-b, High Five and Sf21 cells were infected (96 h, 28° C.) with pFhpa4 or control pF1 viruses and the culture medium incubated with sulfate-labeled ECM. Low Mr HS degradation fragments were released from the ECM only upon incubation with medium conditioned by pFhpa4 infected cells. As shown in FIG. 9, production of these fragments was abolished in the presence of heparin. No heparanase activity was detected in the culture medium of control, non-infected cells. These results indicate that the heparanase enzyme expressed by cells infected with the pFhpa4 virus is capable of degrading HS when complexed to other macromolecular constituents (i.e. fibronectin, laminin, collagen) of a naturally produced intact ECM, in a manner similar to that reported for highly metastatic tumor cells or activated cells of the immune system (10, 76).

### Example 4

### Purification of Recombinant Human Heparanase

The recombinant heparanase was partially purified from medium of pFhpa4 infected Sf21 cells by Heparin-Sepharose chromatography (FIG. 10a) followed by gel filtration of the pooled active fractions over an FPLC Superdex 75 column (FIG. 11a). A ˜63 kDa protein was observed, whose quantity, as was detected by silver stained SDS-polyacrylamide gel ectrophoresis, correlated with heparanase activity in the relevant column fractions (FIGS. 10b and 11b, respectively). This protein was not detected in the culture medium of cells infected with the control pF1 virus and was subjected to a similar fractionation on heparin-Sepharose (not shown).

### Example 5

### Expression of the Human hpa cDNA in Various Cell Types, Organs and Tissues

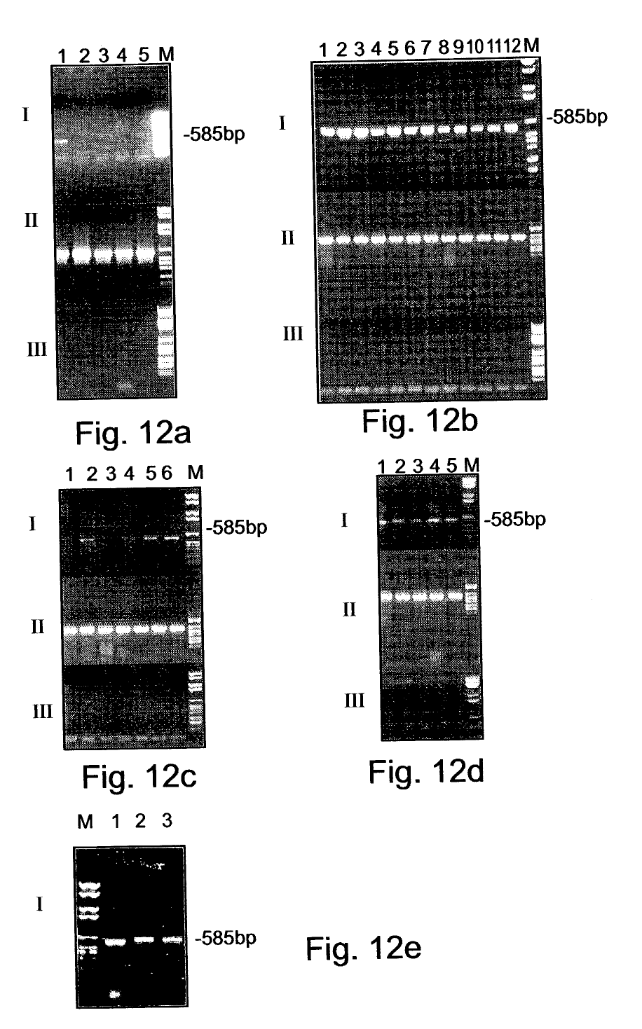

Referring now to FIGS. 12a-e, RT-PCR was applied to evaluate the expression of the hpa gene by various cell types and tissues. For this purpose, total RNA was reverse transcribed and amplified. The expected 585 bp long cDNA was clearly demonstrated in human kidney, placenta (8 and 11 weeks) and mole tissues, as well as in freshly isolated and short termed (1.5-48 h) cultured human placental cytotrophoblastic cells (FIG. 12a), all known to express a high heparanase activity (79). The hpa transcript was also expressed by normal human neutrophils (FIG. 12b). In contrast, there was no detectable expression of the hpa mRNA in embryonic human muscle tissue, thymus, heart and adrenal (FIG. 12b). The hpa gene was expressed by several, but not all, human bladder carcinoma cell lines (FIG. 12c), SK hepatoma (SK-hep-1), ovarian carcinoma (OV 1063), breast carcinoma (435, 231), melanoma and megakaryocytic (DAMI, CHRF) human cell lines (FIGS. 12d-e).

The above described expression pattern of the hpa transcript was determined to be in a very good correlation with heparanase activity levels determined in various tissues and cell types (not shown).

### Example 6

### Isolation of an Extended 5′ End of hpa cDNA from Human SK-hep1 Cell Line

The 5′ end of hpa cDNA was isolated from human SK-hep1 cell line by PCR amplification using the Marathon RACE (rapid amplification of cDNA ends) kit (Clontech). Total RNA was prepared from SK-hep1 cells using the TRI-Reagent (Molecular research center Inc.) according to the manufacturer instructions. Poly A+ RNA was isolated using the mRNA separator kit (Clonetech).

The Marahton RACE SK-hep1 cDNA composite was constructed according to the manufacturer recommendations. First round of amplification was preformed using an adaptor specific primer AP1: 5′-CCATCCTAATACG ACTCACTATAGGGC-3′, SEQ ID NO:1, and a hpa specific antisense primer hpl-629: 5′-CCCCAGGAGCAGCAGCATCAG-3′, SEQ ID NO:17, corresponding to nucleotides 119-99 of SEQ ID NO:9. The resulting PCR product was subjected to a second round of amplification using an adaptor specific nested primer AP2: 5′-ACTCACTATAGGGCTCGAGCGGC-3′, SEQ ID NO:3, and a hpa specific antisense nested primer hpl-666 5′-AGGCTTCGAGCGCAGCAGCAT-3′, SEQ ID NO:18, corresponding to nucleotides 83-63 of SEQ ID NO:9. The PCR program was as follows: a hot start of 94° C. for 1 minute, followed by 30 cycles of 90° C.-30 seconds, 68° C.-4 minutes. The resulting 300 bp DNA fragment was extracted from an agarose gel and cloned into the vector pGEM-T Easy (Promega). The resulting recombinant plasmid was designated pHPSK1.

The nucleotide sequence of the pHPSK1 insert was determined and it was found to contain 62 nucleotides of the 5′ end of the placenta hpa cDNA (SEQ ID NO:9) and additional 178 nucleotides upstream, the first 178 nucleotides of SEQ ID NOs:13 and 15.

A single nucleotide discrepancy was identified between the SK-hep1 cDNA and the placenta cDNA. The “T” derivative at position 9 of the placenta cDNA (SEQ ID NO:9), is replaced by a “C” derivative at the corresponding position 187 of the SK-hep1 cDNA (SEQ ID NO:13).

The discrepancy is likely to be due to a mutation at the 5′ end of the placenta cDNA clone as confirmed by sequence analysis of several additional cDNA clones isolated from placenta, which like the SK-hep1 cDNA contained C at position 9 of SEQ ID NO:9.

The 5′ extended sequence of the SK-hep1 hpa cDNA was assembled with the sequence of the hpa cDNA isolated from human placenta (SEQ ID NO:9). The assembled sequence contained an open reading frame which encodes, as shown in SEQ ID NOs:14 and 15, a polypeptide of 592 amino acids with a calculated molecular weight of 66,407 daltons. The open reading frame is flanked by 93 bp 5′ untranslated region (UTR).

### Example 7

### Isolation of the Upstream Genomic Region of the hpa Gene

The upstream region of the hpa gene was isolated using the Genome Walker kit (Clontech) according to the manufacturer recommendations. The kit includes five human genomic DNA samples each digested with a different restriction endonuclease creating blunt ends: EcoRV, ScaI, DraI, PvuII and SspI.

The blunt ended DNA fragments are ligated to partially single stranded adaptors. The Genomic DNA samples were subjected to PCR amplification using the adaptor specific primer and a gene specific primer. Amplification was performed with Expand High Fidelity (Boehringer Mannheim).

A first round of amplification was performed using the ap1 primer: 5′-G TAATACGACTCACTATAGGGC-3′, SEQ ID NO:19, and the hpa specific antisense primer hpl-666: 5′-AGGCTTCGAGCGCAGCAGCAT-3′, SEQ ID NO:18, corresponding to nucleotides 83-63 of SEQ ID NO:9. The PCR program was as follows: a hot start of 94° C.-3 minutes, followed by 36 cycles of 94° C.-40 seconds, 67° C.-4 minutes.

The PCR products of the first amplification were diluted 1:50. One μl of the diluted sample was used as a template for a second amplification using a nested adaptor specific primer ap2: 5′-ACTATAGGGCACGCGTGGT-3′, SEQ ID NO:20, and a hpa specific antisense primer hpl-690, 5′-CTTGGGCTCACC TGGCTGCTC-3′, SEQ ID NO:21, corresponding to nucleotides 62-42 of SEQ ID NO:9. The resulting amplification products were analyzed using agarose gel electrophoresis. Five different PCR products were obtained from the five amplification reactions. A DNA fragment of approximately 750 bp which was obtained from the SspI digested DNA sample was gel extracted. The purified fragment was ligated into the plasmid vector pGEM-T Easy (Promega). The resulting recombinant plasmid was designated pGHP6905 and the nucleotide sequence of the hpa insert was determined.

A partial sequence of 594 nucleotides is shown in SEQ ID NO:16. The last nucleotide in SEQ ID NO:13 corresponds to nucleotide 93 in SEQ ID:13. The DNA sequence in SEQ ID NO:16 contains the 5′ region of the hpa cDNA and 501 nucleotides of the genomic upstream region which are predicted to contain the promoter region of the hpa gene.

### Example 8

### Expression of the 592 Amino Acids HPA Polypeptide in a Human 293 Cell Line

The 592 amino acids open reading frame (SEQ ID NOs:13 and 15) was constructed by ligation of the 110 bp corresponding to the 5′ end of the SK-hep1 hpa cDNA with the placenta cDNA. More specifically the Marathon RACE-PCR amplification product of the placenta hpa DNA was digested with SacI and an approximately 1 kb fragment was ligated into a SacI-digested pGHP6905 plasmid. The resulting plasmid was digested with EarI and AatII. The EarI sticky ends were blunted and an approximately 280 bp EarI/blunt-AatII fragment was isolated. This fragment was ligated with pFasthpa digested with EcoRI which was blunt ended using Klenow fragment and further digested with AatII. The resulting plasmid contained a 1827 bp insert which includes an open reading frame of 1776 bp, 31 bp of 3′ UTR and 21 bp of 5′ UTR. This plasmid was designated pFastLhpa.

A mammalian expression vector was constructed to drive the expression of the 592 amino acids heparanase polypeptide in human cells. The hpa cDNA was excised prom pFastLhpa with BssHII and NotI. The resulting 1850 bp BssHII-NotI fragment was ligated to a mammalian expression vector pSI (Promega) digested with MluI and NotI. The resulting recombinant plasmid, pSIhpaMet2 was transfected into a human 293 embryonic kidney cell line.

Transient expression of the 592 amino-acids heparanase was examined by western blot analysis and the enzymatic activity was tested using the gel shift assay. Both these procedures are described in length in U.S. Pat. No. 6,177,545, which is incorporated by reference as if fully set forth herein. Cells were harvested 3 days following transfection. Harvested cells were re-suspended in lysis buffer containing 150 mM NaCl, 50 mM Tris pH 7.5, 1% Triton X-100, 1 mM PMSF and protease inhibitor cocktail (Bochringer Mannheim). 40 μg protein extract samples were used for separation on a SDS-PAGE. Proteins were transferred onto a PVDF Hybond-P membrane (Amersham). The membrane was incubated with an affinity purified polyclonal anti heparanase antibody, as described in U.S. Pat. No. 6,177,545. A major band of approximately 50 kDa was observed in the transfected cells as well as a minor band of approximately 65 kDa. A similar pattern was observed in extracts of cells transfected with the pShpa as demonstrated in U.S. Pat. No. 6,177,545. These two bands probably represent two forms of the recombinant heparanase protein produced by the transfected cells. The 65 kDa protein probably represents a heparanase precursor, while the 50 kDa protein is suggested herein to be the processed or mature form.

The catalytic activity of the recombinant protein expressed in the pShpaMet2 transfected cells was tested by gel shift assay. Cell extracts of transfected and of mock transfected cells were incubated overnight with heparin (6 μg in each reaction) at 37° C., in the presence of 20 mM phosphate citrate buffer pH 5.4, 1 mM CaCl₂, 1 mM DTT and 50 mM NaCl. Reaction mixtures were then separated on a 10% polyacrylamide gel. The catalytic activity of the recombinant heparanase was clearly demonstrated by a faster migration of the heparin molecules incubated with the transfected cell extract as compared to the control. Faster migration indicates the disappearance of high molecular weight heparin molecules and the generation of low molecular weight degradation products.

### Example 9

### Chromosomal Localization of the hpa Gene

Chromosomal mapping of the hpa gene was performed utilizing a panel of monochromosomal human/CHO and human/mouse somatic cell hybrids, obtained from the UK HGMP Resource Center (Cambridge, England).

40 ng of each of the somatic cell hybrid DNA samples were subjected to PCR amplification using the hpa primers: hpu565 5′-AGCTCTGTAGATGTGC TATACAC-3′, SEQ ID NO:22, corresponding to nucleotides 564-586 of SEQ ID NO:9 and an antisense primer hpl171 5′-GCATCTTAGCCGTCTTTCTTCG-3′, SEQ ID NO:23, corresponding to nucleotides 897-876 of SEQ ID NO:9.

The PCR program was as follows: a hot start of 94° C.-3 minutes, followed by 7 cycles of 94° C.-45 seconds, 66° C.-1 minute, 68° C.-5 minutes, followed by 30 cycles of 94° C.-45 seconds, 62° C.-1 minute, 68° C.-5 minutes, and a 10 minutes final extension at 72° C.

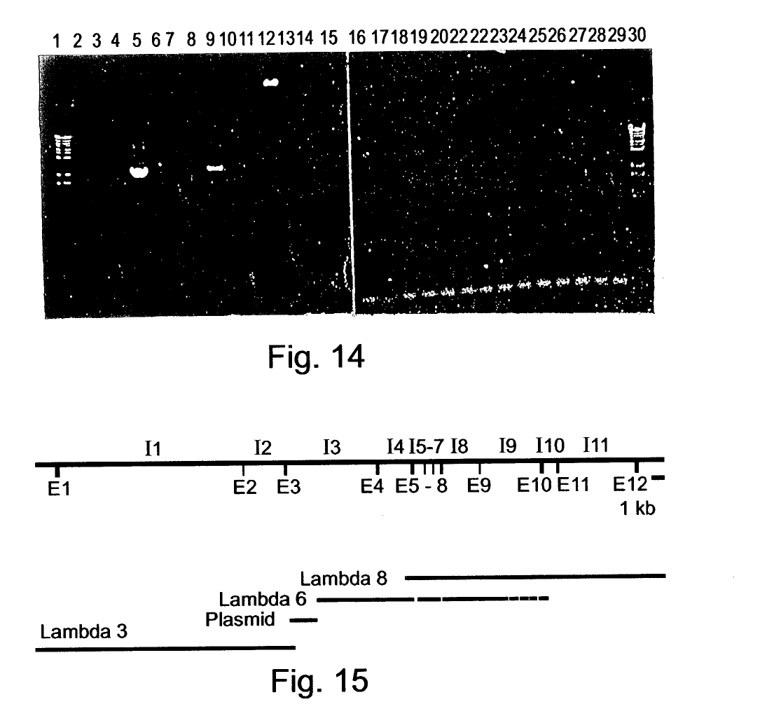

The reactions were performed with Expand long PCR (Boehringer Mannheim). The resulting amplification products were analyzed using agarose gel electrophoresis. As demonstrated in FIG. 14, a single band of approximately 2.8 Kb was obtained from chromosome 4, as well as from the control human genomic DNA. A 2.8 kb amplification product is expected based on amplification of the genomic hpa clone (data not shown). No amplification products were obtained neither in the control DNA samples of hamster and mouse nor in somatic hybrids of other human chromosome.

### Example 10

### Human Genomic Clone Encoding Heparanase

Five plaques were isolated following screening of a human genomic library and were designated L3-1, L5-1, L8-1, L10-1 and L6-1. The phage DNAs were analyzed by Southern hybridization and by PCR with hpa specific and vector specific primers. Southern analysis was performed with three fragments of hpa cDNA: a PvuIl-BamHI fragment (nucleotides 32-450, SEQ ID NO:9), a BamHI-NdeI fragment (nucleotides 451-1102, SEQ ID NO:9) and an NdeI-XhoI fragment (nucleotides 1103-1721, SEQ ID NO:9).

Following Southern analysis, phages L3, L6, L8 were selected for further analysis. A scheme of the genomic region and the relative position of the three phage clones is depicted in FIG. 15. A 2 kb DNA fragment containing the gap between phages L6 and L3 was PCR amplified from human genomic DNA with two gene specific primers GHpuL3 and GHpIL6. The PCR product was cloned into the plasmid vector pGEM-T-easy (Promega).

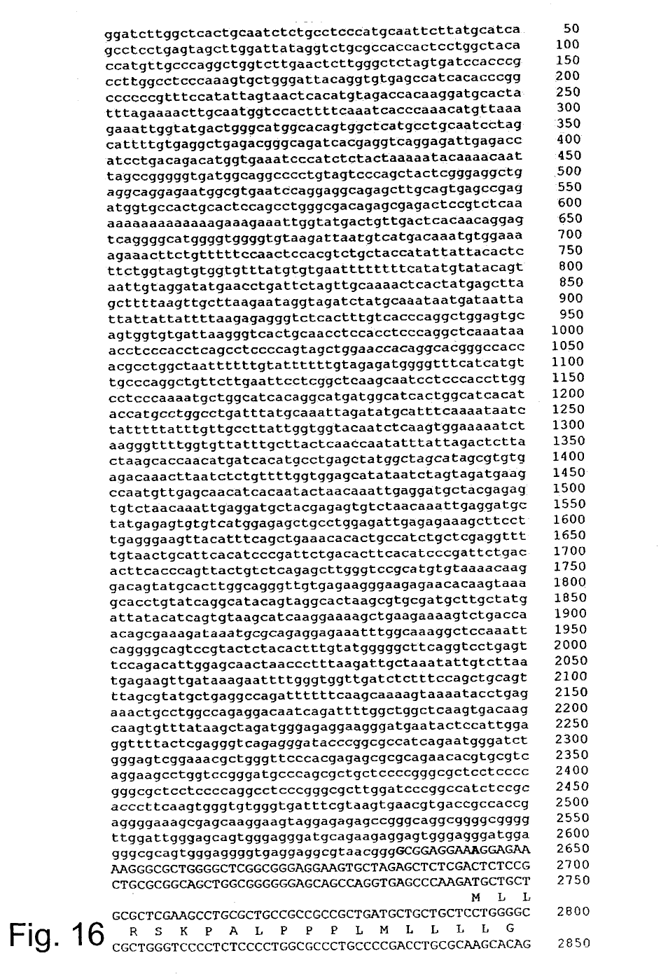

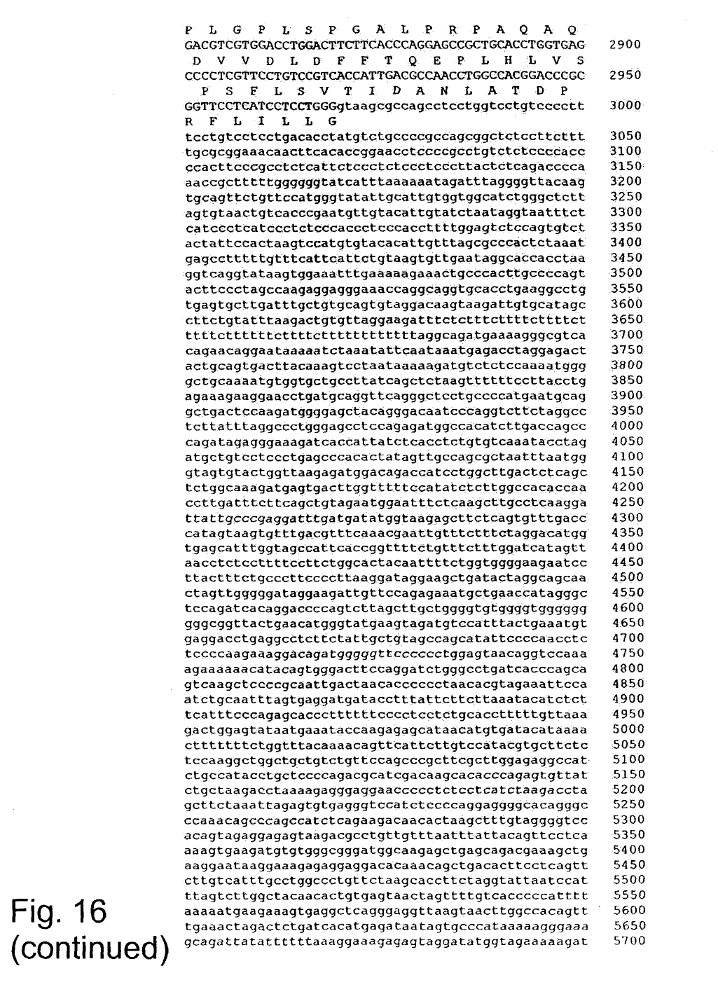

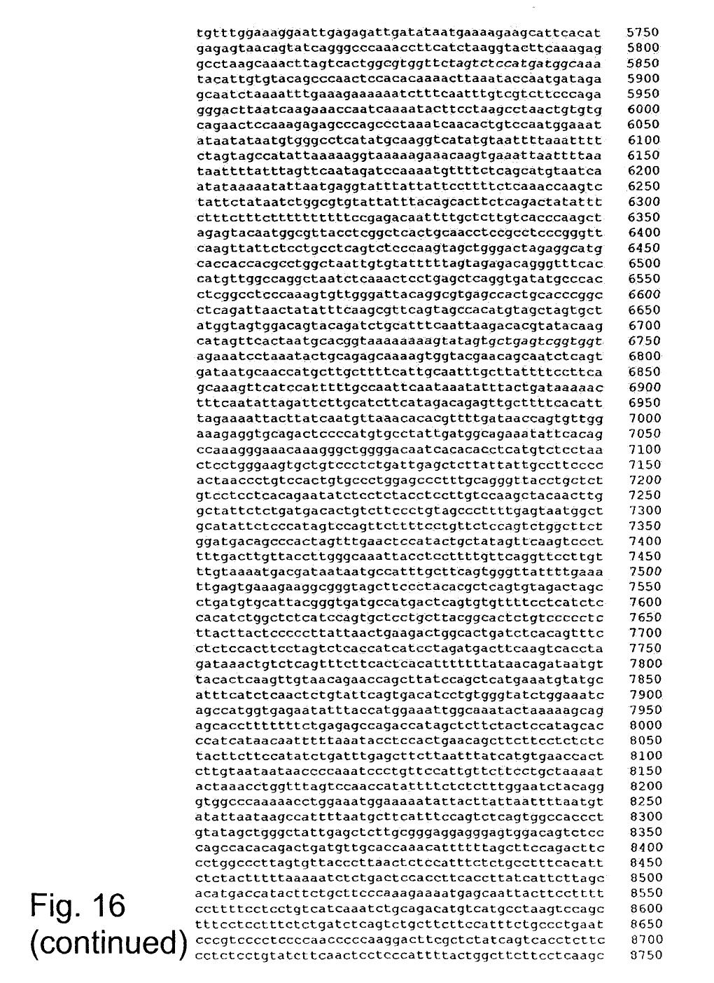

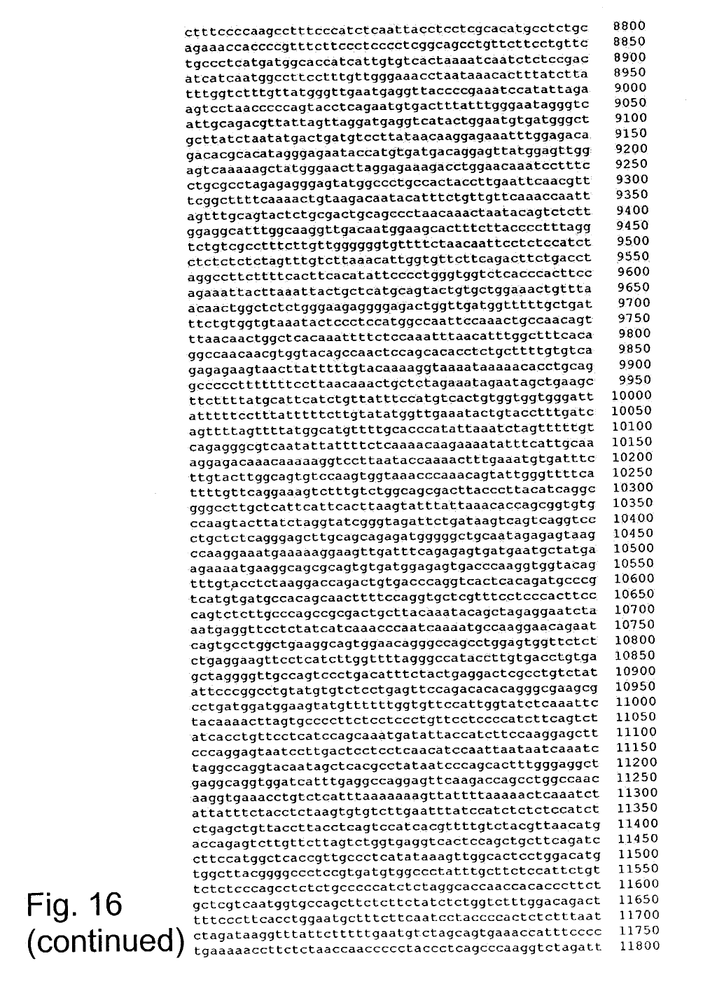

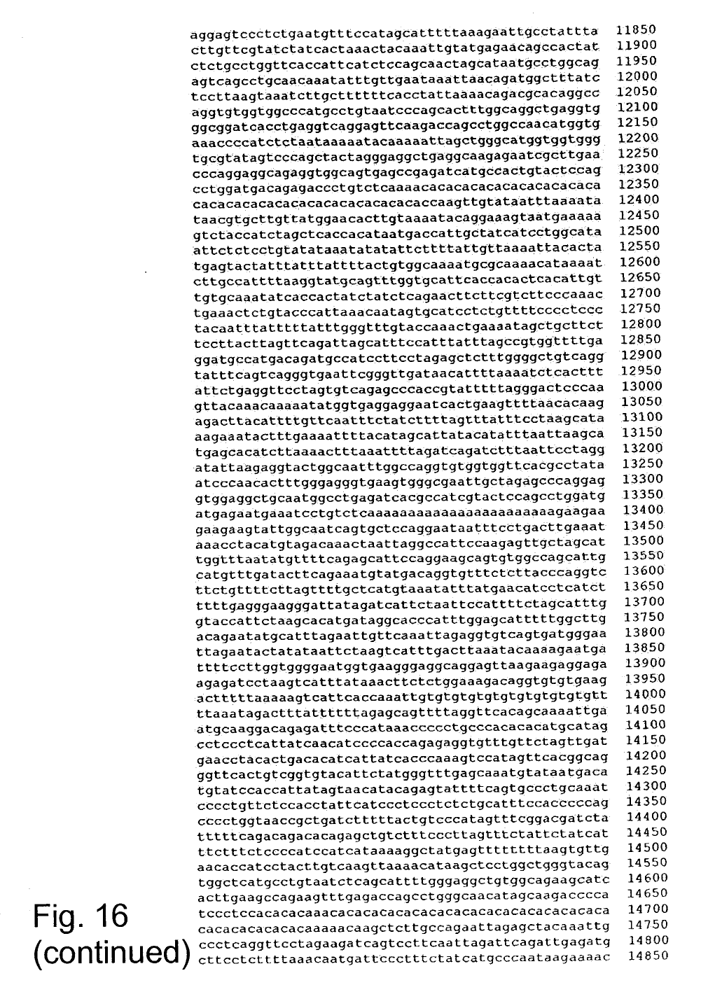

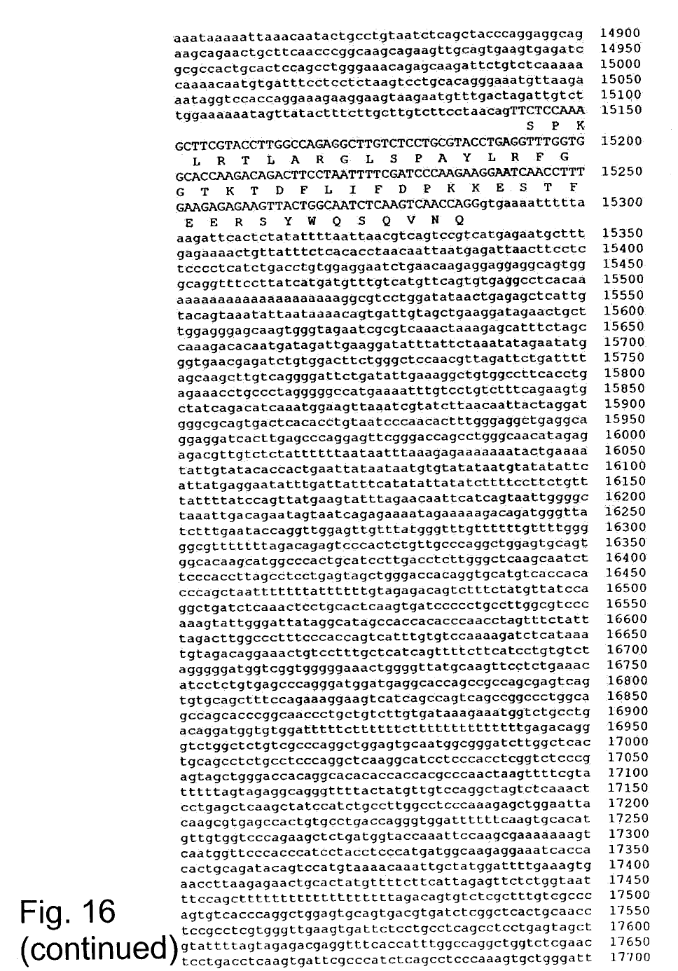

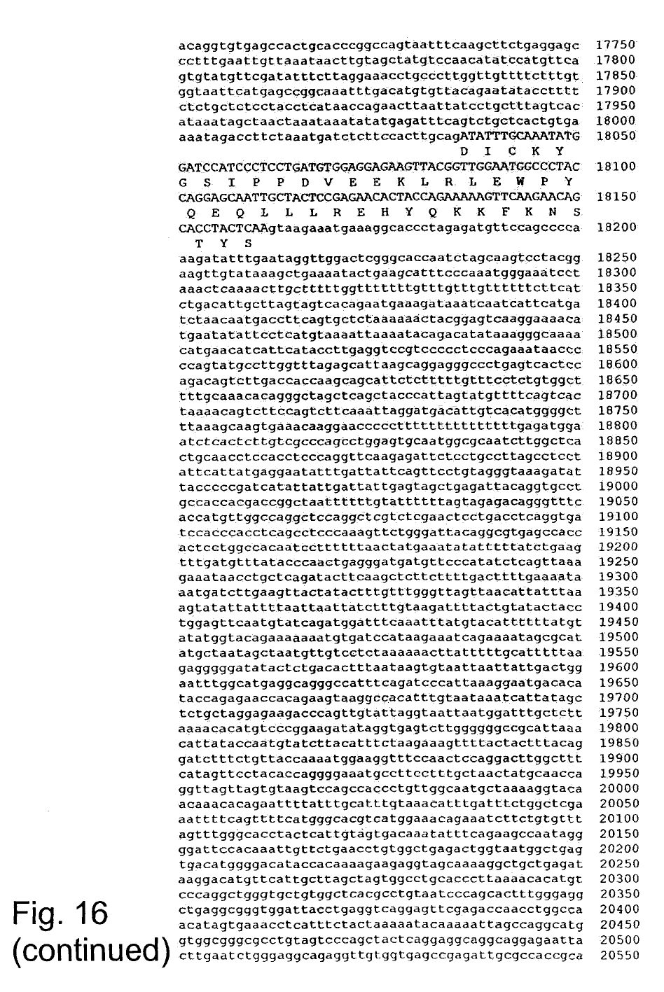

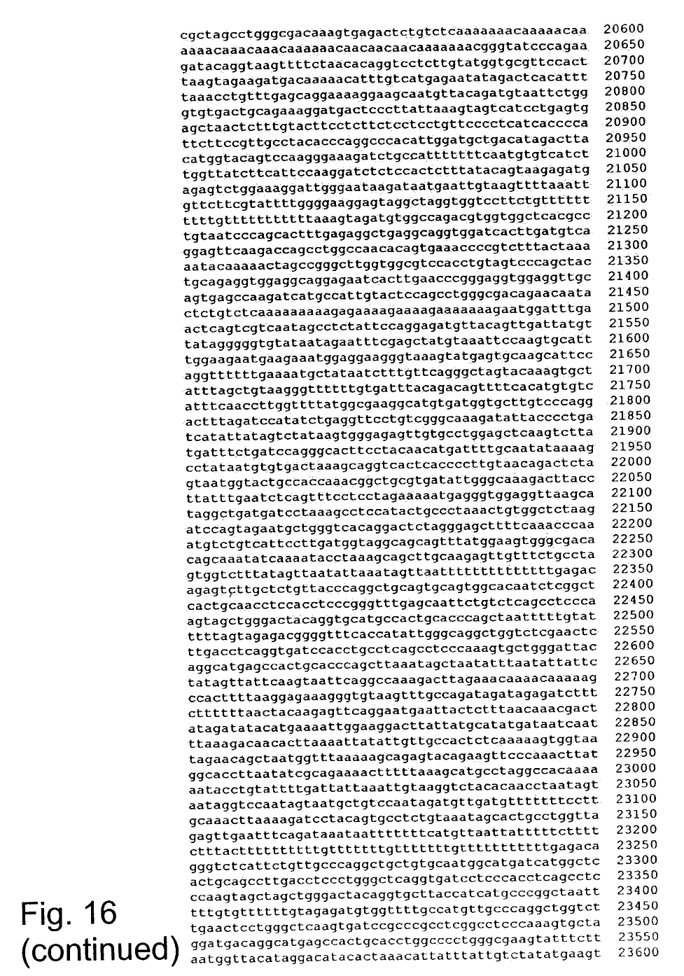

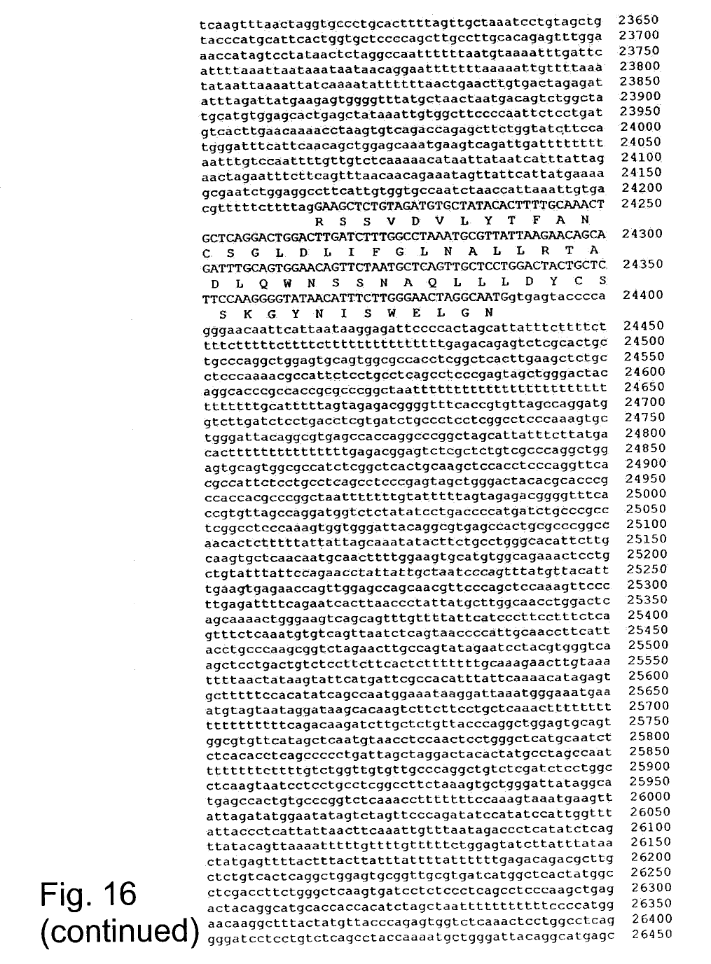

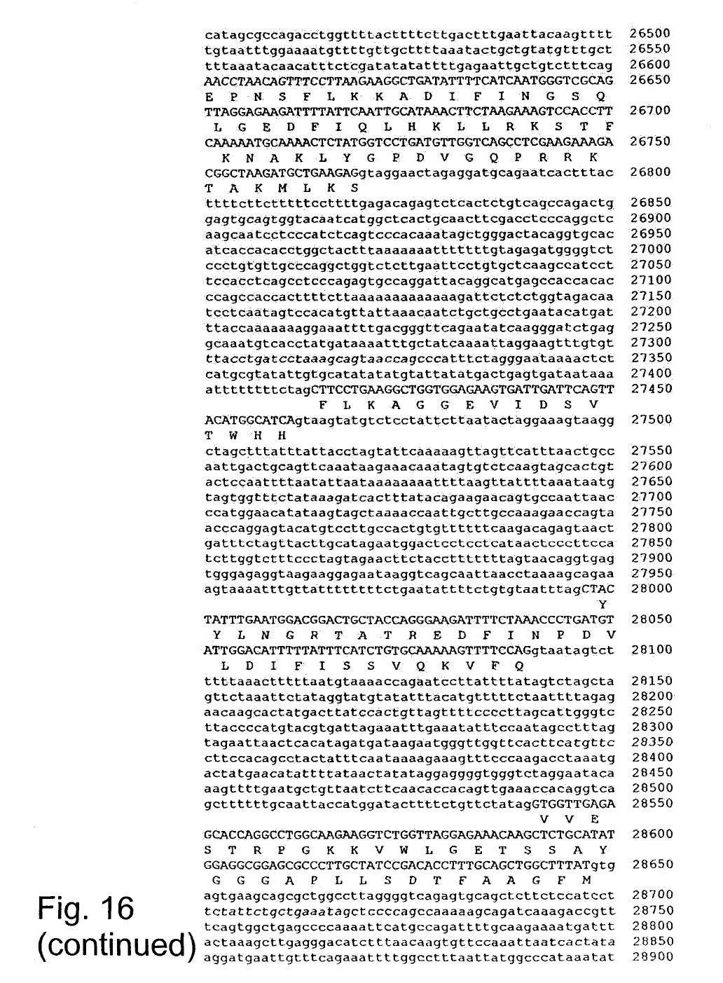

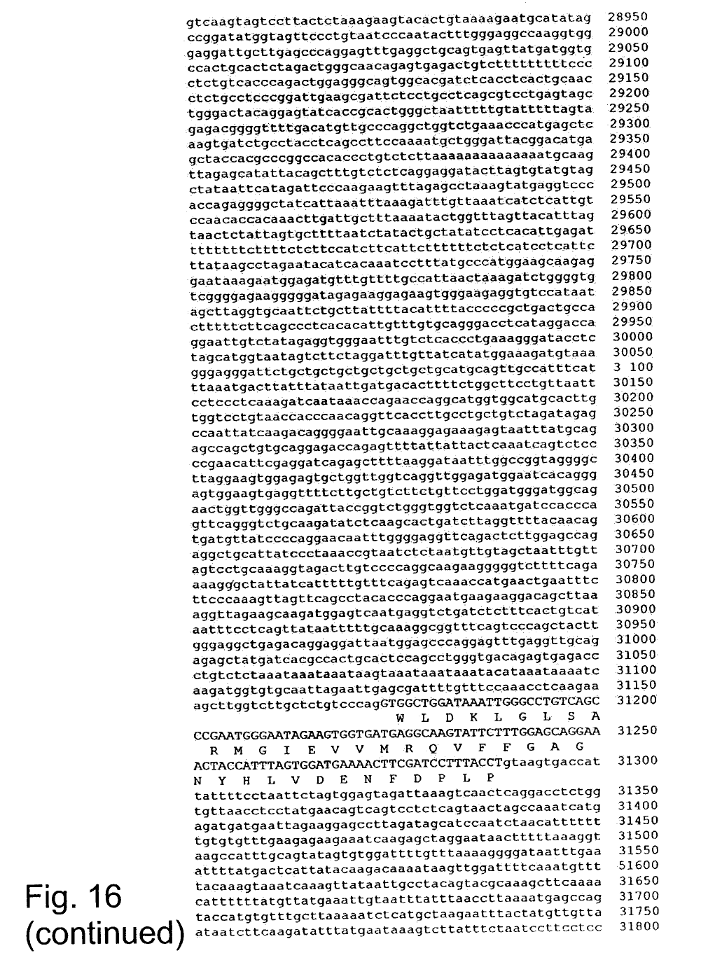

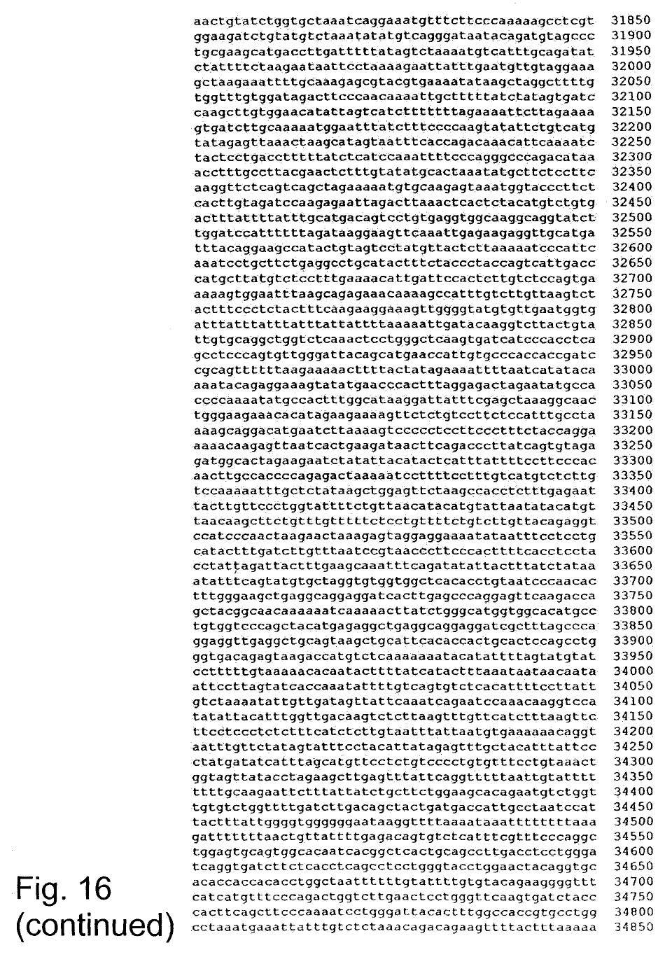

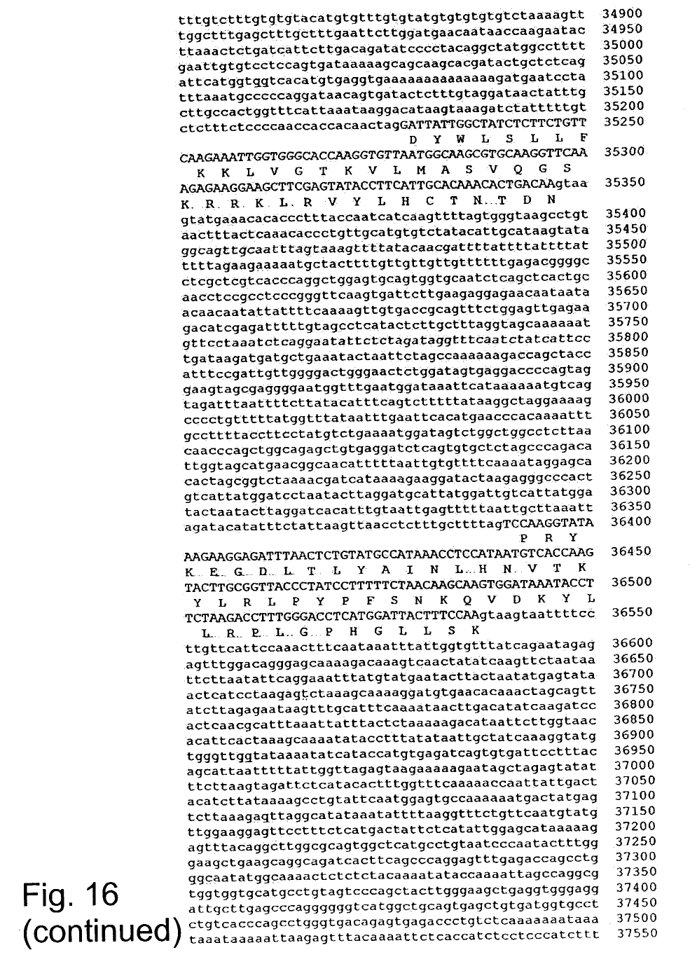

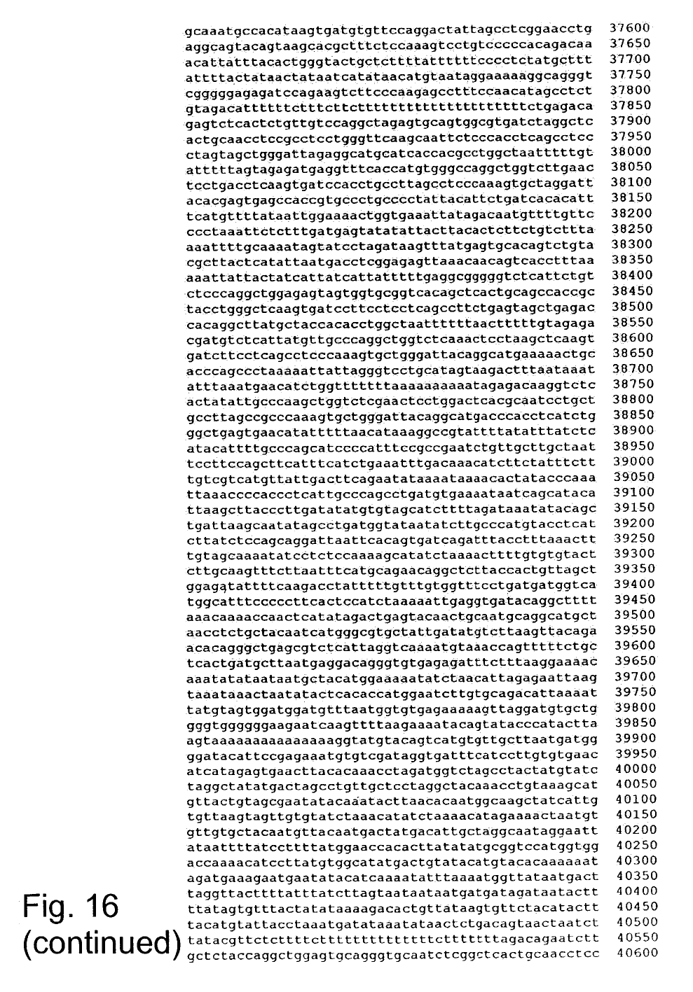

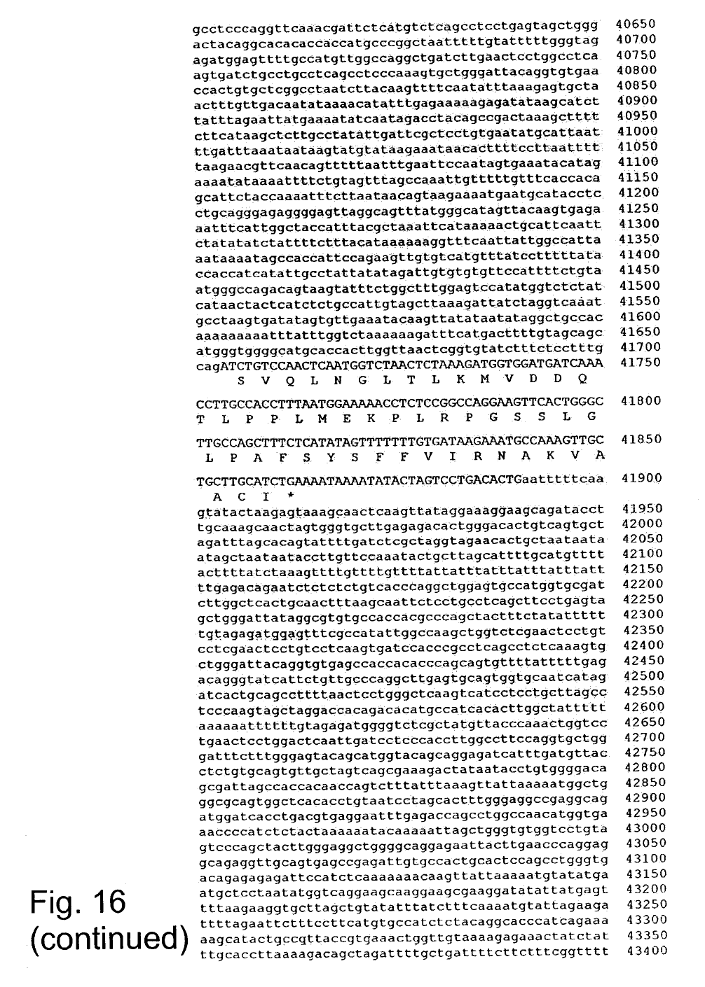

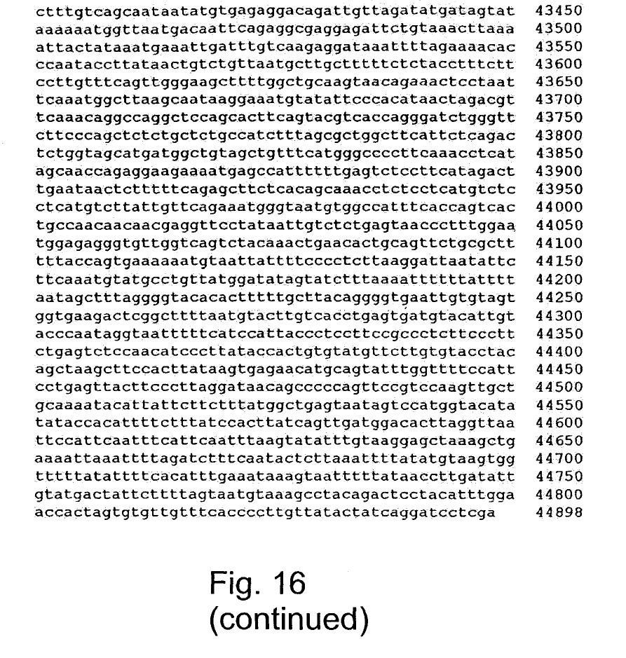

Large scale DNA sequencing of the three Lambda clones and the amplified fragment was performed with Lambda purified DNA by primer walking. A nucleotide sequence of 44,898 bp was analyzed (FIG. 16, SEQ ID NO:42). Comparison of the genomic sequence with that of hpa cDNA revealed 12 exons separated by 11 introns (FIGS. 15 an 16). The genomic organization of the hpa gene is depicted in FIG. 15 (top). The sequence include the coding region from the first ATG to the stop codon which spans 39,113 nucleotides, 2742 nucleotides upstream of the first ATG and 3043 nucleotides downstream of the stop codon. Splice site consensus sequences were identified at exon/intron junctions.

### Example 11

### Alternative Splicing

Several minor RT-PCR products were obtained from various cell types, following amplification with hpa specific primers. Each one found to contain a deletion of one or two exons. Some of these PCR products contain ORFs, which encode potential shorter proteins.

Table 1 below summarizes the alternative spliced products isolated from various cell lines.

Fragments of similar sizes were obtained following amplification with two cell lines, placenta and platelets.

| Cell type | Nucleotides deleted | Exons deleted | ORF |
|---|---|---|---|
| Platelets | 1047-1267 | 8, 9 | + |
| Platelets | 1154-1267 | 9 | − |
| Platelets | 289-435, 562-735 | 2, 4 | − |
| Sk-hep1, platelets, Zr75 | 562-735 | 4 | + |
| Sk-hep1 (hepatoma) | 561-904 | 4, 5 | − |
| Zr75 (breast carcinoma) | 96-203 | 1 (partial) | + |

### Example 12

### Mouse and Rat hpa

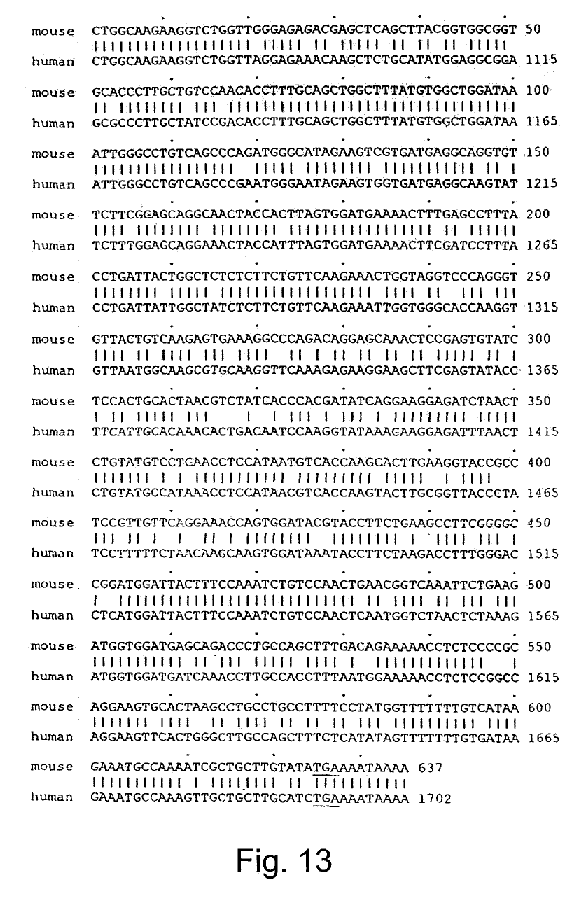

EST databases were screened for sequences homologous to the hpa gene. Three mouse EST's were identified (accession No. Aa177901, from mouse spleen, Aa067997 from mouse skin, Aa47943 from mouse embryo), assembled into a 824 bp cDNA fragment which contains a partial open reading frame (lacking a 5′ end) of 629 bp and a 3′ untranslated region of 195 bp (SEQ ID NO:12). As shown in FIG. 13, the coding region is 80% similar to the 3′ end of the hpa cDNA sequence. These EST's are probably cDNA fragments of the mouse hpa homolog that encodes for the mouse heparanase.

Searching for consensus protein domains revealed an amino terminal homology between the heparanase and several precursor proteins such as Procollagen Alpha 1 precursor, Tyrosine-protein kinase-RYK, Fibulin-1, Insulin-like growth factor binding protein and several others. The amino terminus is highly hydrophobic and contains a potential trans-membrane domain. The homology to known signal peptide sequences suggests that it could function as a signal peptide for protein localization.

The amino acid sequence of human heparanase was used to search for homologous sequences in the DNA and protein databases. Several human EST's were identified, as well as mouse sequences highly homologous to human heparanase. The following mouse EST's were identified AA177901, AA674378, AA67997, AA047943, AA690179, AI122034, all sharing an identical sequence and correspond to amino acids 336-543 of the human heparanase sequence. The entire mouse heparanase cDNA was cloned, based on the nucleotide sequence of the mouse EST's. PCR primers were designed and a Marathon RACE was performed using a Marathon cDNA library from 15 days mouse embryo (Clontech) and from BL6 mouse melanoma cell line. The mouse hpa homologous cDNA was isolated following several amplification steps. A 1.1 kb fragment was amplified from mouse embryo Marathon cDNA library. The first cycle of amplification was performed with primers mhpl773 and Ap1 and the second cycle with primers mhpl736 and AP2. A 1.1 kb fragment was then amplified from BL6 Marathon cDNA library. The first cycle of amplification was performed with the primers mhpl152 and Ap1, and the second with mhpl83 and AP2. The combined sequence was homologous to nucleotides 157-1702 of the human hpa cDNA, which encode amino acids 33-543. The 5′ end of the mouse hpa gene was isolated from a mouse genomic DNA library using the Genome Walker kit (Clontech). An 0.9 kb fragment was amplified from a DraI digested Genome walker DNA library. The first cycle of amplification was performed with primers mhpl114 and Ap1 and the second with primers mhpl103 and AP2. The assembled sequence (SEQ ID NOs:43, 45) is 2396 nucleotides long. It contains an open reading frame of 1605 nucleotides, which encode a polypeptide of 535 amino acids (SEQ ID NOs:44, 45), 196 nucleotides of 3′ untranslated region (UTR), and anupstream sequence which includes the promoter region and the 5′-UTR of the mouse hpa cDNA.. According to two promoter predicting programs TSSW and TSSG, the transcription start site is localized to nucleotide 431 of SEQ ID NOs:43, 45, 163 nucleotides upstream of the first ATG codon. The 431 upstream genomic sequence contains the promoter region. A TATA box is predicted at position 394 of SEQ ID NOs:43, 45. The mouse and the human hpa genes share an average homology of 78% between the nucleotide sequences and 81% similarity between the deduced amino acid sequences.

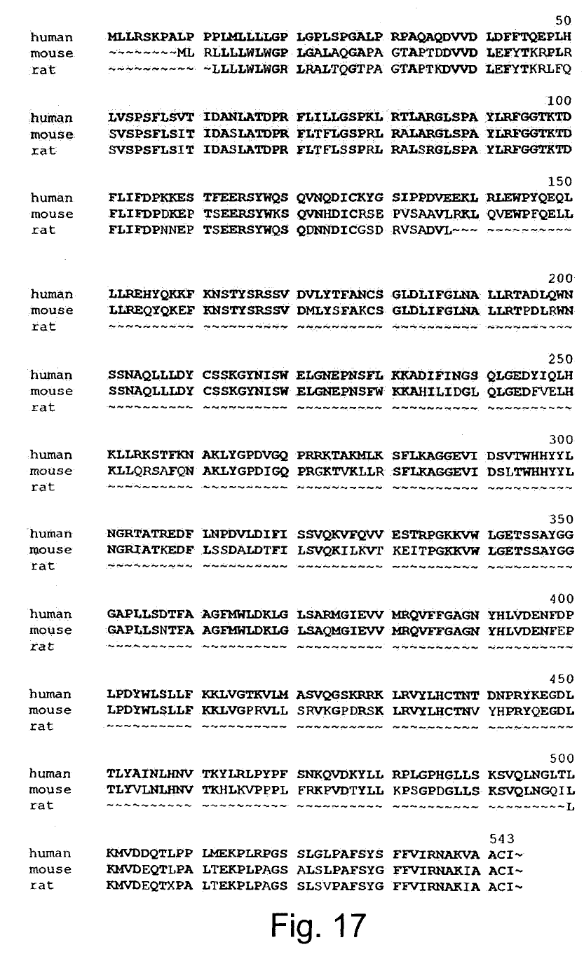

Search for hpa homologous sequences, using the Blast 2.0 server revealed two EST's from rat: AI060284 (385 nucleotides, SEQ ID NO:46) which is homologous to the amino terminus (68% similarity to amino acids 12-136) of human heparanase and AI237828 (541 nucleotides, SEQ ID NO:47) which is homologous to the carboxyl terminus (81% similarity to amino acids 500-543) of human heparanase, and contains a 3′-UTR. A comparison between the human heparanase and the mouse and rat homologous sequences is demonstrated in FIG. 17.

### Example 13

### Prediction of Heparanase Active Site

Homology search of heparanase amino acid sequence against the DNA and the protein databases revealed no significant homologies. The protein secondary structure as predicted by the PHD program consists of alternating alpha helices and beta sheets. The fold recognition server of UCLA predicted alpha/beta barrel structure, with under-threshold confidence.

Five of 15 proteins, which were predicted to have most similar folds, were glycosyl hydrolases from various organisms: 1xyza—xylanase from Clostridium Thermocellum, 1pbga—6-phospho-beta-δ-galactosidase from Lactococcus Lactis, 1amy—alpha-amylase from Barley, 1ecea—endocellulase from Acidothermus Cellulolyticus and 1qbc—hexosaminidase alpha chain, glycosyl hydrolase.

Protein homology search using the bioaccelerator pulled out several proteins, including glycosyl hydrolyses such as beta-fructofuranosidase from Vicia faba (broad bean) and from potato, lactase phlorizin hydrolase from human, xylanases from Clostridium thermocellum and from Streptomyces halstedii and cellulase from Clostridium thermocellum. Blocks 9.3 database pulled out the active site of glycosyl hydrolases family five, which includes cellulases from various bacteria and fungi. Similar active site motif is shared by several lysosomal acid hydrolases (63) and other glycosyl hydrolases. The common mechanism shared by these enzymes involves two glutamic acid residues, a proton donor and a nucleophile.

Despite the lack of an overall homology between the heparanase and other glycosyl hydolases, the amino acid couple Asp-Glu (NE), which is characteristic of the proton donor of glycosyl hydrolyses of the GH-A clan, was found at positions 224-225 of the human heparanase protein sequence. As in other clan members, this NE couple is located at the end of a β sheet.

Considering the relative location of the proton donor and the predicted secondary structure, the glutamic acid that functions as nucleophile is most likely located at position 343, or at position 396. Identification of the active site and the amino acids directly involved in hydrolysis opens the way for expression of the defined catalytic domain. In addition, it will provide the tools for rational design of enzyme activity either by modification of the microenviroment or catalytic site itself.

### Example 14

### Expression of hpa Antisense in Mammalian Cell Lines

A mammalian expression vector Hpa2Kepcdna3 was constructed in order to express hpa antisense in mammalian cells. hpa cDNA (1.7 kb EcoRI fragment) was cloned into the plasmid pCDNA3 in 3′>5′ (antisense) orientation. The construct was used to transfect MBT2-T50 and T24P cell lines. 2×10⁵ cells in 35 mm plates were transfected using the Fugene protocol (Boehringer Mannheim). 48 hours after transfection cells were trypsinized and seeded in six well plates. 24 hours later G418 was added to initiate selection. The number of colonies per 35 mm plate following 3 weeks:

| | Antisense | No insert |
|---|---|---|
| T24P | 15 | 60 |
| MBT-T50 | 1 | 6 |

The lower number of colonies obtained after transfection with hpa antisense, as compared with the control plasmid suggests that the introduction of hpa antisense interfere with cell growth. This experiment demonstrates the use of complementary antisense hpa DNA sequence to control heparanase expression in cells. This approach may be used to inhibit expression of heparanase in vivo, in, for example, cancer cells and in other pathological processes in which heparanase is involved.

### Example 15

### Zoo Blot

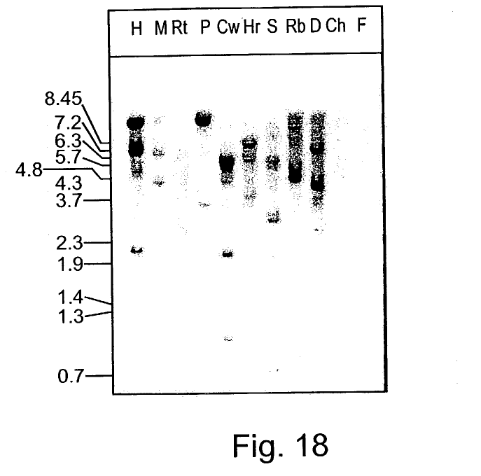

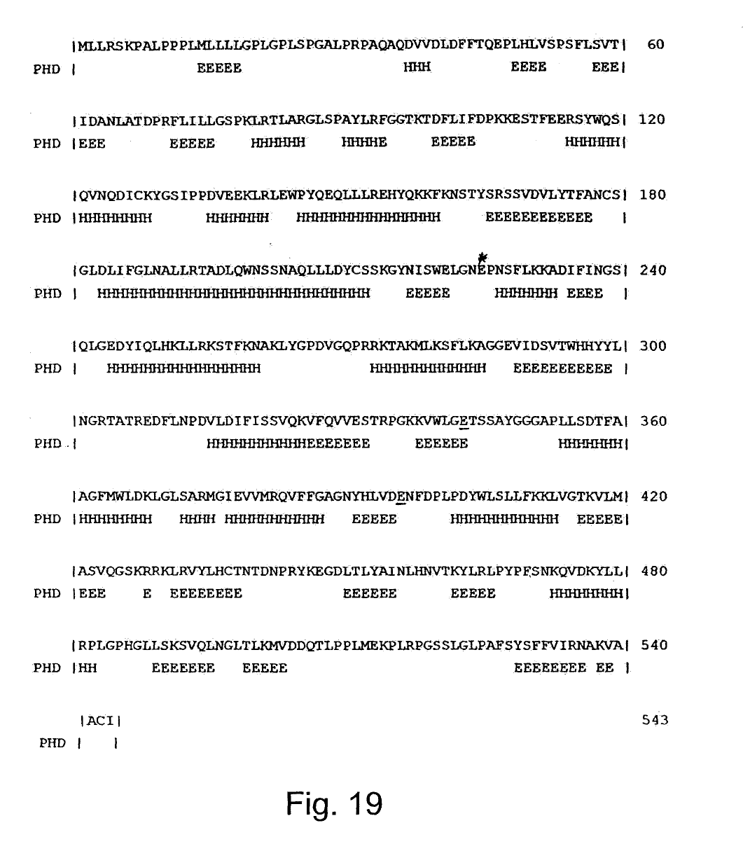

Hpa cDNA was used as a probe to detect homologous sequences in human DNA and in DNA of various animals. The autoradiogram of the Southern analysis is presented in FIG. 18. Several bands were detected in human DNA, which correlated with the accepted pattern according to the genomic hpa sequence. Several intense bands were detected in all mammals, while faint bands were detected in chicken. This correlates with the phylogenetic relation between human and the tested animals. The intense bands indicate that hpa is conserved among mammals as well as in more genetically distant organisms. The multiple bands patterns suggest that in all animals, like in human, the hpa locus occupy large genomic region. Alternatively, the various bands could represent homologous sequences and suggest the existence of a gene family, which can be isolated based on their homology to the human hpa reported herein. This conservation was actually found, between the isolated human hpa cDNA and the mouse homologue.

### Example 16

### Characterization of the hpa Promoter

The DNA sequence upstream of the hpa first ATG was subjected to computational analysis in order to localize the predicted transcription start site and to identify potential transcription factors binding sites. Recognition of human PolII promoter region and start of transcription were predicted using the TSSW and TSSG programs. Both programs identified a promoter region upstream of the coding region. TSSW pointed at nucleotide 2644 and TSSG at 2635 of SEQ ID NO:42. These two predicted transcription start sites are located 4 and 13 nucleotides upstream of the longest hpa cDNA isolated by RACE.

A hpa promoter-GFP reporter vector was constructed in order to investigate the regulation of hpa transcription. Two constructs were made, containing 1.8 kb and 1.1 kb of the hpa promoter region. The reporter vector was transfected into T50-mouse bladder carcinoma cells. Cells transfected with both constructs exhibited green fluorescence, which indicated the promoter activity of the genomic sequence upstream of the hpa-coding region. This reporter vector, enables the monitoring of hpa promoter activity, at various conditions and in different cell types and to characterize the factors involved regulation of hpa expression.

### Example 17

### Human Heparanase Expressing Transgenic Mice

Materials, Methods and Experimetal Results

Immunohistochemistry:

Micrometer sections were deparaffinized and rehydrated. Tissue was then denatured for 3 minutes in a microwave oven in citrate buffer (0.01 M, pH 6.0). Blocking steps included successive incubations in 0.2% glycine, 3% H₂O₂ in methanol and 5% goat serum. Sections were incubated with a monoclonal anti-human heparanase antibody HP-130 (see U.S. Pat. No. 6,177,545) diluted in PBS, or with DMEM supplemented with 10% horse serum as control, diluted as above, followed by incubation with HRP conjugated goat anti mouse IgG+IgM antibody (Jackson). Color was developed using Zymed AEC substrate kit (Zymed) for 10 minutes, followed by counter stain with Mayer's hematoxylin.

Preparation of Dishes Coated with ECM:

Bovine corneal EC were cultured as described in U.S. Pat. No. 5,986,822 except that 5% dextran T-40 was included in the growth medium and the cells were maintained without addition of bFGF for 12 days. The subendothelial ECM was exposed by dissolving the cell layer with PBS containing 0.5% Triton X-100 and 20 mM NH40H, followed by four washed in PBS. The ECM remained intact, free of cellular debris and firmly attached to the entire area of the tissue culture dish. For preparation of sulfate-labeled ECM, corneal endothelial cells were cultured in the presence of Na₂[³⁵S]O₄ (Amersham) added (25 μCi/ml) one day and 5 days after seeding and the cultures were incubated with the label without medium change. Ten to twelve days after seeding, the cell monolayer was dissolved and the ECM exposed.

Heparanase Activity:

Degradation of sulfate labeled ECM by heparanase was determined as described in U.S. Pat. No. 5,986,822. Briefly, ECM was incubated (24 hours, 37° C., pH 6.2) with recombinant heparanase or hpa-transfected cells and sulfate labeled material released into the incubation medium was analyzed by gel filtration on a Sepharose 6B column. Intact HSPGs were eluted just after the void volume (Kav<0.2, peak I) and HS degradation fragments eluted with 0.5<Kav<0.8 (peak II).

Generation of Heparanase Transgenic Mice:

Human hpa cDNA was cloned from a human placenta cDNA library (see U.S. Pat. No. 5,968,822) using back-translated DNA sequences corresponding to peptides from human hepatoma haparanase. After filling in missing 5′ ends in the placenta EST clones a cDNA fragment, 1721 bp long (GeneBank Accession No. AF144325), contained an open reading frame which encodes a polypeptide of 543 amino acids (GenBank Accession No. AAD41342) with a calculated molecular weight of 61,192 daltons was obtained. High-level constitutive expression of heparanase was driven by chicken beta-actin promoter. The plasmid pCAGGS (64) was modified to contain a unique EcoRI site at position 1719. An XbaI-EcoRI 1.7 kb fragment, which contained the entire open reading frame of heparanase was cloned into the compatible sites of the vector.

Before injection, the plasmid pCAGGS-hpa was digested with SalI and PstI in order to isolate the expression cassette and eliminate bacterial DNA sequences. The resulting fragment contained the CMV-IE enhancer, chicken β-actin promoter and hpa cDNA followed by a rabbit b-globin poly adenylation site.

The DNA fragment containing the hpa expression cassette was injected into fertilized eggs, derived from C57BL×BalbC breed. The isolation of fertilized eggs, injection of DNA and transplantation of blastocytes were conducted by the Department of cell biochemistry—the transgenic unit at the Hadassah Medical School, Jerusalem according to a protocol adapted from Hogan et al. Manipulating the Mouse Embryo A Laboratory Manual, Cold Spring Harbor Laboratory Press, 1994.

Mice developed from the injected blastocytes were tested for the presence of the human hpa transgene in their genome. Genomic DNA was extracted from tail tips of the mice and the human hpa transgene sequence was amplified using human hpa specific PCR primers. To this end, tail fragments were incubated overnight at 55° C. in a lysis buffer (8 M urea, 0.2 M Tris-HCl, 0.4 M NaCl, 20 mM EDTA, 1% N-Laurylsarcosine, 10 μg/ml proteinase K). The dissolved tissue underwent phenol extraction and ethanol precipitation, to obtain a highly purified genomic DNA.

The integration of the human heparanase cDNA in the mouse genome was verified by PCR using two sets of primers. The first couple was designed to amplify the 5′ region of the transgene. It included a β-actin promoter specific primer (designated 5′-pCAGGs) 5′-ATAGGCAGCTGACCTGA-3′ (SEQ ID NO:48) and human hpa specific primer: (designated Hpl-300) 5′-TGACTTGAGATTGCCAGTAACTTC-3′ (SEQ ID NO:49). The second primers set was designed to amplify the 3′ region of the transgene. It included a human hpa specific primer (designated Hpu-830) 5′-CTGTCCAACTCAATGGTCTAACTC-3′ (SEQ ID NO:50), and a primer specific to the plasmid derived 3′-untranslated region (designated 3′ pCAGGS) 5′-TCTAGAGCCTCTGCTAACCA-3′ (SEQ ID NO:51); PCR conditions were as follows: 2 minutes at 95° C. followed by 33 cycles of 15 seconds at 95° C., 1 minute at 58° C. and 1 minute at 72° C.

Four G₀ founder mice were obtained, harboring the human hpa cDNA in their genome as revealed by a PCR reaction specific for the human hpa cDNA. Founders were mated with C57B1 mice to create F1 mice and those were mated among themselves to create F2 mice. Homozygous F2 mice from each G₀ line were identified by Southern blot analysis and a quantitative PCR assay. Homozygosity was verified by mating with C57B1 mice, where all the pups were positive heterozygous. All founder transgenic mice were back crossed with C57BL mice in order to establish C57B1 transgenic mice with a pure genetic background.

Expression of Human Heparanase in Transgenic Mice:

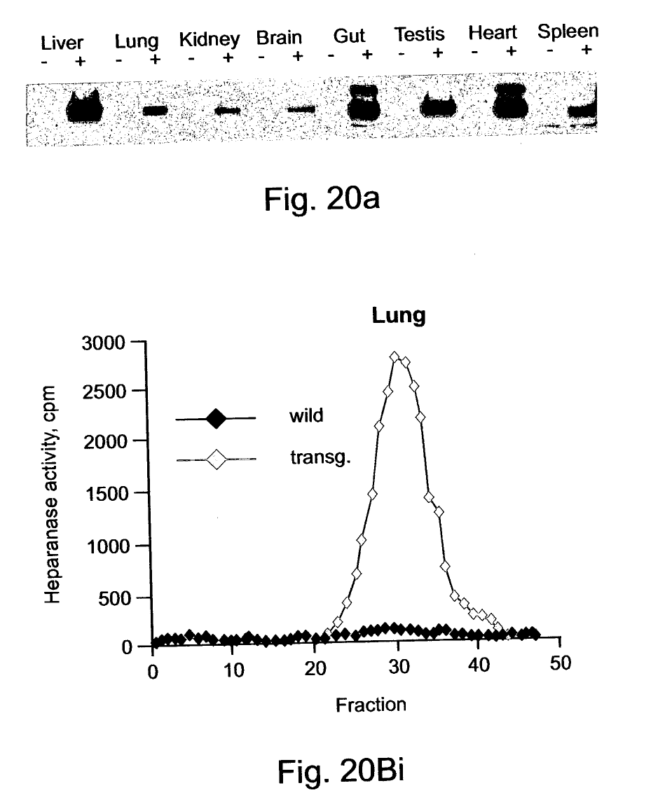

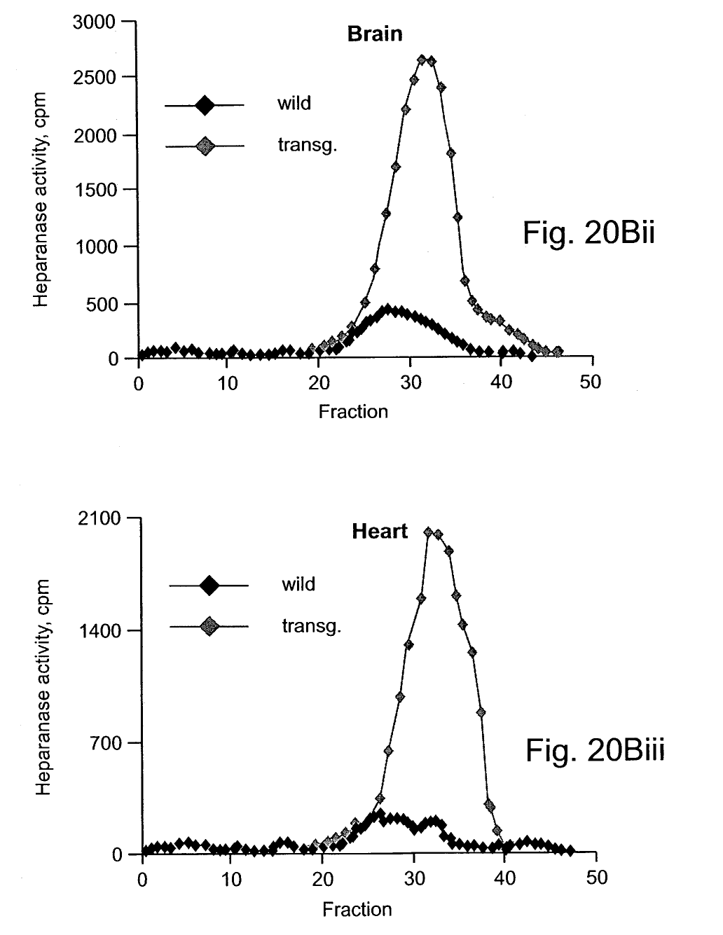

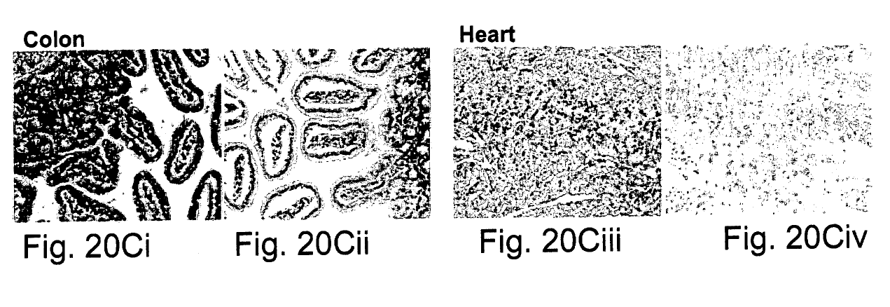

Expression of the heparanase protein was demonstrated by Western blot analysis of tissue extracts derived from F1 transgenic and control mice (FIG. 20A). Measurements of heparanase activity in tissue extracts revealed a much higher activity in the transgenic as compared to control mice in all tissues examined (FIGS. 20Bi-iii). Immunohistochemical staining of tissue sections revealed a strong expression of the human heparanase protein in tissues derived from the transgenic mice, but not control mice (FIGS. 20Ci-iv).

Phenotype of Human Heparanase Overexpressing Transgenic Mice:

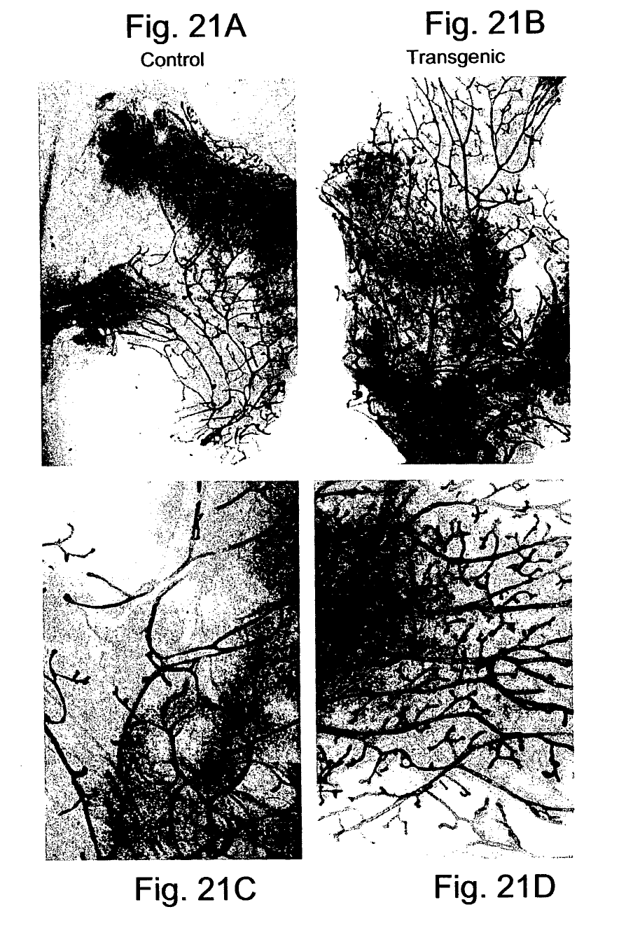

The transgenic mice are fertile and show no apparent signs of abnormality. Few phenotypic alterations were however noted. For example, the virgin transgenic mice develop lobular-alveoli structures in the mammary gland, a phenomenon that is characteristic of mammary glands of pregnant mice (FIGS. 21A-D).

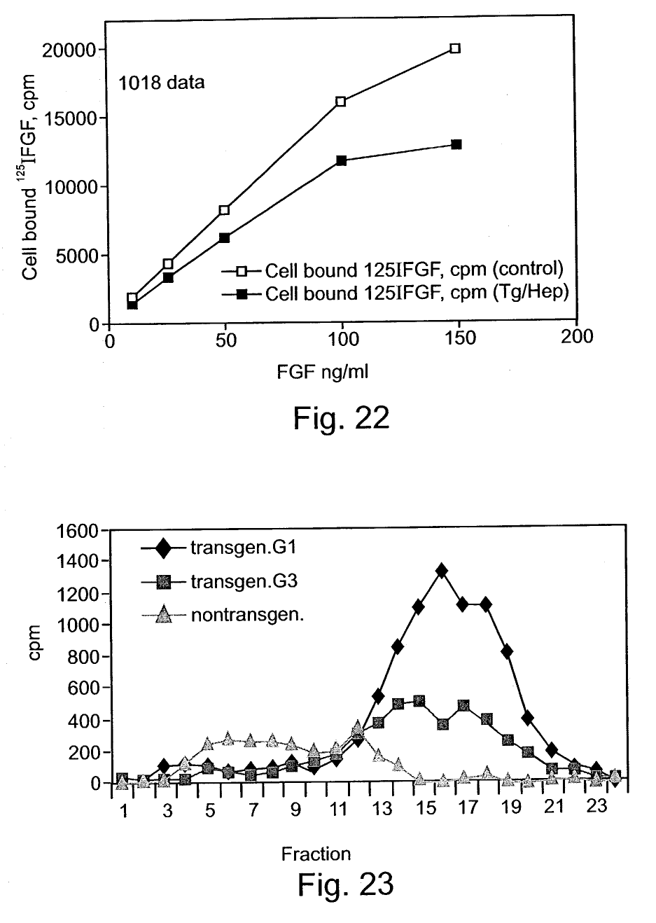

Overexpression of heparanase may lead to alterations in the amount and composition of heparan sulfate in the extracellular matrix (ECM) and surface of cells derived from the transgenic vs. control mice. In order to examine the effect of heparanase overexpression on cell surface heparan sulfate, the bFGF binding capacity of embryonic cells from transgenic and control mice was tested. Fibroblasts were isolated from embryos of transgenic mice and control mice 15 days post gestation. Cells were cultured in DMEM/RPMI/F-12 medium supplemented with 10% FCS. Confluent cells were incubated with various concentrations of radio-iodinated bFGF. Following incubation cells were washed and the bound bFGF was quantitated. As shown in FIG. 22, binding of bFGF to fibroblasts of transgenic embryos was lower than to fibroblasts of control embryos. This observation suggests that high levels of heparanase reduce the amount of heparan sulfate on the cell surface.

Heparanase in Milk of Transgenic Mice:

Milk of transgenic mice was tested for heparanase activity. Milk was obtained from females of two independent lines of transgenic mice and from control mice 7-10 days after delivery. Milk was diluted 1:10 in phosphate citrate buffer pH 6.0 and incubated on 35S labeled ECM for 48 hours. Degradation products were size fractionated. As shown in FIG. 23 heparanase activity was detected in the two transgenic lines G1 and G3, while no activity was detected in milk of control mice. This observation indicates that active heparanase can by produced in the mammary glands and secreted into the milk of transgenic animals.

Tissue Specific Expression of Heparanase in Transgenic Mice:

In more recent experiments, the hpa cDNA was cloned into a PES7 plasmid, a derivative of pSP72 containing the minimal apoA1 promoter, driving expression of the human 7 alpha-hydroxylase enzyme exclusively in the liver of male mice. (PES7 expression vector was a gift from Schayek E., Bresbow L. B, The Rockefeller University NY. The 7 alpha-hydroxylase was replaced by the hpa cDNA in the proper orientation. Briefly, hpa cDNA was excised from pCAGGS-hpa2 using XbaI. The 1.7 kb XbaI fragment was subcloned into the XbaI site of PES7 plasmid. The appropriate linear fragment was cut, purified and subjected to microinjection. A single transgenic mouse expressing the human hpa cDNA was obtained. This mouse was bred to produce F1 mice.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by a Genbank accession number mentioned in this specification are herein incorporated in their entirety by reference into the specification, to the same extent as if each individual publication, patent, patent application or sequence was specifically and individually indicated to be incorporated herein by reference. In addition, citation or identification of any reference in this application shall not be construed as an admission that such reference is available as prior art to the present invention.

### References Cited

### Additional References are Cited in the Text

1. Durand, P., Lehn, P., Callebaut, I., Fabrega, S., Henrissat, B. and Mornon, J. P. (1997). Active-site motifs of lysosomal acid hydrolases: invariant features of clan GH-A glycosyl hydrolases deduced from hydrophobic cluster analysis. Glycobiology, 7(2), 277-284.

2. Vlodavsky, I., Friedmann, Y., Elkin, M., Aingorn, H., Atzmon, R., Ishai-Michaeli, R., Bitan, M., Pappo, O., Peretz, T., Michal, I., Spector, L and Pecker I. (1999). Mammalian heparanase: gene cloning, expression and function in tumor progression and metastasis. Nature Medicine (In press)

3. Wight, T. N., Kinsella, M. G., and Qwarnstromn, E. E. (1992). The role of proteoglycans in cell adhesion, migration and proliferation. Curr. Opin. Cell Biol., 4, 793-801.

4. Jackson, R. L., Busch, S. J., and Cardin, A. L. (1991). Glycosaminoglycans: Molecular properties, protein interactions and role in physiological processes. Physiol. Rev., 71, 481-539.

5. Wight, T. N. (1989). Cell biology of arterial proteoglycans. Arteriosclerosis, 9, 1-20.

6. Kjellen, L., and Lindahl, U. (1991). Proteoglycans: structures and interactions. Annu. Rev. Biochem., 60, 443-475.

7. Ruoslahti, E., and Yamaguchi, Y. (1991). Proteoglycans as modulators of growth factor activities. Cell, 64, 867-869.

8. Vlodavsky, I., Bar-Shavit, R., Korner, G., and Fuks, Z. (1993). Extracellular matrix-bound growth factors, enzymes and plasma proteins. In Basement membranes: Cellular and molecular aspects (eds. D. H. Rohrbach and R. Timpl), pp 327-343. Academic press Inc., Orlando, Fla.

9. Vlodavsky, I., Bar-Shavit, R., Korner, G., and Fuks, Z. (1993). Extracellular matrix-bound growth factors, enzymes and plasma proteins. In Basement membranes: Cellular and molecular aspects (eds. D. H. Rohrbach and R. Timpl), pp 327-343. Academic press Inc., Orlando, Fla.

10. Vlodavsky, I., Eldor, A., Haimovitz-Friedman, A., Matzner, Y., Ishai-Michaeli, R., Levi, E., Bashkin, P., Lider, O., Naparstek, Y., Cohen, I. R., and Fuks, Z. (1992). Expression of heparanase by platelets and circulating cells of the immune system: Possible involvement in diapedesis and extravasation. Invasion & Metastasis, 12, 112-127.

11. Nakaji, M., Irimura, T., and Nicolson, G. L. (1988). Heparanase and tumor metastasis. J. Cell. Biochem., 36, 157-167.

12. Vlodavsky, I. et al. Invasion Metastasis 1995, 14:290-302.

13. Nakagima, M. et al J. Cell. Biochem. 1988, 36:157-167.

14. Vlodavsky, I. et al. Cancer res. 1983, 43:2704-2711.

15. Vlodavsky, I. et al. J. Med 1988, 24:464-470.

16. Vlodavsky, I. et al. Invasion and Metastasis 12:112-127.

17. Gilat, D. et al. J. Exp. Med. 1995, 181:1929-1934.

18. Matzner et al. 1985, J. Clin. Invest. 10:1306-1313.

19. Mollinedo, F. et al. Biochem. J. 1997, 327:917-923.

20. Murphy, G. et al. Biochem. J. 1990, 192:517-525.

21. Nakajima, M. et al. J. Cell. Biochem. 1988, 36(2):157-167.

22. Ishai-Michaeli R. wt al. Cell Reg. 1990, 1:833-842.

23. Cardon-Cardo C. et al. Lab. Inrest. 1990, 63:832-840.

24. Nicolson, G. L. (1988). Organ specificity of tumor metastasis: Role of preferential adhesion, invasion and growth of malignant cells at specific secondary sites. Cancer Met. Rev., 7, 143-188.

25. Liotta, L. A., Rao, C. N., and Barsky, S. H. (1983). Tumor invasion and the extracellular matrix. Lab. Invest., 49, 639-649.

26. Vlodavsky, I., Fuks, Z., Bar-Ner, M., Ariav, Y., and Schirrmacher, V. (1983). Lymphoma cell mediated degradation of sulfated proteoglycans in the subendothelial extracellular matrix: Relationship to tumor cell metastasis. Cancer Res., 43, 2704-2711.

27. Sun L, Feusi E, Sibalic A, Beck-Schimmer B, Wuthrich R P (1998). Expression profile of hyaluronidase mRNA transcripts in the kidney and in renal cells. Kidney Blood Press Res;21(6):413-8

28. Parish, C. R., Coombe, D. R., Jakobsen, K. B., and Underwood, P. A. (1987). Evidence that sulphated polysaccharides inhibit tumor metastasis by blocking tumor cell-derived heparanase. Int. J. Cancer, 40, 511-517.

29. Burgess, W. H., and Maciag, T. (1989). The heparin-binding (fibroblast) growth factor family of proteins. Annu. Rev. Biochem., 58, 575-606.

30. Folkman, J., and Klagsbrun, M. (1987). Angiogenic factors. Science, 235, 442-447.

31. Vlodavsky, I., Folkman, J., Sullivan, R., Fridman, R., Ishai-Michaelli, R., Sasse, J., and Klagsbrun, M. (1987). Endothelial cell-derived basic fibroblast growth factor: Synthesis and deposition into subendothelial extracellular matrix. .Proc. Natl. Acad. Sci. USA, 84, 2292-2296.

32. Folkman, J., Klagsbrun, M., Sasse, J., Wadzinski, M., Ingber, D., and Vlodavsky, I. (1980). A heparin-binding angiogenic protein—basic fibroblast growth factor—is stored within basement membrane. Am. J. Pathol., 130, 393-400.

33. Bashkin, P., Doctrow, S., Klagsbrun, M., Svahn, C. M., Folkman, J., and Vlodavsky, I. (1989). Basic fibroblast growth factor binds to subendothelial extracellular matrix and is released by heparitinase and heparin-like molecules. Biochemistry, 28, 1737-1743.

34. Ishai-Michaeli, R., Svahn, C.-M., Chajek-Shaul, T., Korner, G., Ekre, H.-P., and Vlodavsky, I. (1992). Importance of size and sulfation of heparin in release of basic fibroblast factor from the vascular endothelium and extracellular matrix. Biochemistry, 31, 2080-2088.

35. Ishai-Michaeli, R., Eldor, A., and Vlodavsky, I. (1990). Heparanase activity expressed by platelets, neutrophils and lymphoma cells releases active fibroblast growth factor from extracellular matrix. Cell Reg., 1, 833-842.

36. Vlodavsky, I., Bar-Shavit, R., Ishai-Michaeli, R., Bashkin, P., and Fuks, Z. (1991). Extracellular sequestration and release of fibroblast growth factor: a regulatory mechanism? Trends Biochem. Sci., 16, 268-271.

37. Vlodavsky, I., Bar-Shavit, R., Korner, G., and Fuks, Z. (1993). Extracellular matrix-bound growth factors, enzymes and plasma proteins. In Basement membranes: Cellular and molecular aspects (eds. D. H. Rohrbach and R. Timpl), pp327-343. Academic press Inc., Orlando, Fla.

38. Yayon, A., Klagsbrun, M., Esko, J. D., Leder, P., and Ornitz, D. M. (1991). Cell surface, heparin-like molecules are required for binding of basic fibroblast growth factor to its high affinity receptor. Cell, 64, 841-848.

39. Spivak-Kroizman, T., Lemmon, M. A., Dikic, I., Ladbury, J. E., Pinchasi, D., Huang, J., Jaye, M., Crumley, G., Schlessinger, J., and Lax, I. (1994). Heparin-induced oligomerization of FGF molecules is responsible for FGF receptor dimerizatio, activation, and cell proliferation. Cell, 79, 1015-1024.

40. Ornitz, D. M., Herr, A. B., Nilsson, M., West, a., J., Svahn, C.-M., and Waksman, G. (1995). FGF binding and FGF receptor activation by synthetic heparan-derived di- and trisaccharides. Science, 268, 432-436.

41. Gitay-Goren, H., Soker, S., Vlodavsky, I., and Neufeld, G. (1992). Cell surface associated heparin-like molecules are required for the binding of vascular endothelial growth factor (VEGF) to its cell surface receptors. J. Biol. Chem., 267, 6093-6098.

42. Ernst S, Langer R, Cooney C L, Sasisekharan R. (1995). Enzymatic degradation of glycosaminoglycans. Crit Rev Biochem Mol Biol; 30(5):387-444

43. Current protocols in molecular biology (1994-1999). Ausubel, F. M., Brent, R., Kingston, R. E., Moore D. D., Seidman, J. G., Smith, J. A., Struhl, K. Eds. John Wiley & Sons, Inc.

44. Rapraeger, A., Krufka, A., and Olwin, B. R. (1991). Requirement of heparan sulfate for bFGF-mediated fibroblast growth and myoblast differentiation. Science, 252, 1705-1708.

45. Shieh, M-T., Wundunn, D., Montgomery, R. I., Esko, J. D., and Spear, P. G. J. (1992). Cell surface receptors for herpes simplex virus are heparan sulfate proteoglycans. J Cell Biol., 116, 1273-1281.

46. Chen, Y., Maguire, T., Hileman, R. E., Fromm, J. R., Esko, J. D., Linhardt, R. J., and Marks, R. M. (1997). Dengue virus infectivity depends on envelope protein binding to target cell heparan sulfate. Nature Medicine 3, 866-871.

47. Putnak, J. R., Kanesa-Thasan, N., and Innis, B. L. (1997). A putative cellular receptor for dengue viruses. Nature Medicine 3, 828-829.

48. Narindrasorasak, S., Lowery, D., Gonzalez-DeWhitt, P., Poorman, R. A., Greenberg, B., Kisilevsky, R. (1991). High affinity interactions between the Alzheimer's beta-amyloid precursor protein and the basement membrane form of theparan sulfate proteoglycan. J. Biol. Chem., 266, 12878-83.

49. Eisenberg, S., Sehayek, E., Olivecrona, T., and Vlodavsky, I. (1992). Lipoprotein lipase enhances binding of lipoproteins to heparan sulfate on cell surfaces and extracellular matrix. J. Clin. Invest., 90, 2013-2021.

50. Ross, R. (1993). The pathogenesis of atherosclerosis: a perspective for the 1990s. Nature (Lond.)., 362:801-809.

51. Zhong-Sheng, J., Walter, J., Brecht, R., Miranda, D., Mahmood Hussain, M., Innerarity, T. L. and Mahley, W. R. (1993). Role of heparan sulfate proteoglycans in the binding and uptake of apolipoprotein E-enriched remnant lipoproteins by cultured cells. J. Biol. Chem., 268, 10160-10167.

52. Current protocols in protein science (1996-1999). Coligan, J. E., Dunn, B. M., Ploegh, H. L., Speicher, D. W. and Wingfield, P. T. Eds. John Wiley & Sons Inc.

53. Freeman C and Parish C R (1998) Human platelet heparanase: purification, characterization and catalytic activity.: Biochem J March 15;330 (Pt 3):1341-50.

54. Kussie et al. (1999) Biochem Biophys Res Commun. 261(1):183-7.

55. Hullet et al. (1999) Nature Medicine 5(7):803-809.

56. Toyoshima and Nakajima (1999). J. Biol. Chem. 274(34):24153-24160

57. Fairbanks et al. (1999) J. Biol. Chem. 274(42):29587-29590

58. Friedmann et al. (2000) Expression of heparanase in normal, dysplastic, and neoplastic human colonic mucosa and stroma: evidence for its role in colonic tumorigenesis. Am J Pathol. October, 2000;157(4):1167-75

59. Capecchi, M., Cell 22:479-488 (1980).

60. Gordon, J. W. et al. (Proc. Natl. Acad. Sci. USA 77:7380-7384 (1978)

61. Wagner, T. E. et al., Proc. Natl. Acad. Sci. USA 78:6376-6380 (1981)

62. Grosschedl, R. et al. Cell October 1984,38(3):647-58

63. Dempsey, L. A. et al. Trends Biochem Sci. August 2000;25(8):349-51

64. Niwa, H et al. Gene 108: 193-200, (1991)

65. Clark A J, J Mammary Gland Biol Neoplasia July 1998;3(3):337-50

66. Jiang et al. J Biol Chem Jan. 5, 2001;276(1):593-600

67. Zaidi et al. J Clin Invest April 1999;103(8):1211-9

68. Mirault M E et al. Ann N Y Acad Sci Nov. 17, 1994;738:104-15

69. Guy et al. Proc. Natl. Acad. Sci. USA 1992, 89:10578-82

70. Tomoyuki Shirai et al. Mutation research 2000, 412:219-226

71. Hogan et al. Manipulating the Mouse Embryo A Laboratory Manual, Cold Spring Harbor Laboratory Press, 1994

72. Gospodarowicz, D., Mescher, A L., Birdwell, C R. (1977). Stimulation of corneal endothelial cell proliferation in vitro by fibroblast and epidermal growth factors. Exp Eye Res 25, 75-89.

73. Vlodavsky, I., Liu, G. M., and Gospodarowicz, D. (1980). Morphological appearance, growth behavior and migratory activity of human tumor cells maintained on extracellular matrix vs. plastic. Cell, 19, 607-616.

74. Gospodarowicz, D., Delgado, D., and Vlodavsky, I. (1980). Permissive effect of the extracellular matrix on cell proliferation in-vitro. Proc. Natl. Acad. Sci. USA., 77, 4094-4098.

75. Haimovitz-Friedman, A., Falcone, D. J., Eldor, A., Schirrmacher, V., Vlodavsky, I., and Fuks, Z. (1991) Activation of platelet heparitinase by tumor cell-derived factors. Blood, 78, 789-796.

76. Vlodavsky, I., Mohsen, M., Lider, O., Ishai-Michaeli, R., Ekre, H.-P., Svahn, C. M., Vigoda, M., and Peretz, T. (1995). Inhibition of tumor metastasis by heparanase inhibiting species of heparin. Invasion & Metastasis, 14, 290-302.

77. Savitsky, K., Platzer, M., Uziel, T., Gilad, S., Sartiel, A., Rosental, A., Elroy-Stein, O., Siloh, Y. and Rotman, G. (1997). Ataxia-telangiectasia: structural diversity of untranslated sequences suggests complex post-translational regulation of ATM gene expression. Nucleic Acids Res. 25(9), 1678-1684.

78. Vlodavsky, I., Ishai-Michaeli, R., Bar-Ner, M., Fridman, R., Horowitz, A. T., Fuks, Z. and Biran, S. (1988). Involvement of heparanase in tumor metastasis and angiogenesis. Is. J. Med., 24, 464-470.

79. Goshen, R., Hochberg, A., Korner, G., Levi, E., Ishai-Michaeli, R., Elkin, M., de Grot, N., and Vlodavsky, I. (1996). Purification and characterization of placental heparanase and its expression by cultured cytotrophoblasts. Mol. Human Reprod., 2, 679-684.

What is claimed is:

1. A transgenic non-human animal whose genome comprises an exogenous polynucleotide sequence integrated into said genome, said exogenous polynucleotide sequence including a promoter active in tissues of the non-human, and a region encoding a human heparanase, wherein said promoter and said region encoding human heparanase are operably linked in said exogenous polynucleotide such that human heparanase is expressed in at least a portion of the cells of the non-human animal.

2. The transgenic non-human animal of claim 1, being homozygous for said exogenous polynucleotide sequence.

3. The transgenic non-human animal of claim 1, being heterozygous for said exogenous polynucleotide sequence.

4. The transgenic non-human animal of claim 1, having a single locus harboring said exogenous polynucleotide sequence.

5. The transgenic non-human animal of claim 1, having at least two loci each harboring said exogenous polynucleotide sequence.

6. The transgenic non-human animal of claim 1, wherein said human heparanase is genetically modified to be cleavable into an active form via a protease.

7. The transgenic non-human animal of claim 1, wherein said heparanase is processed by an endogenous protease of the non-human animal into an active form.

8. The transgenic non-human animal of claim 1, wherein said region of said exogenous polynucleotide sequence encodes an active form of heparanase.

9. The transgenic non-human animal of claim 1, being a mammal.

10. The transgenic non-human animal of claim 1, being an avian.

11. The transgenic non-human animal of claim 1, wherein said exogenous polynucleotide sequence includes a tissue specific promoter for directing expression of said heparanase in a tissue specific manner.

12. The transgenic non-human animal of claim 1, wherein said promoter is a constitutive promoter for directing expression of said heparanase in constitutive manner.

13. The transgenic non-human animal of claim 1, wherein said promoter is an inducible promoter for directing expression of said heparanase in an inducible manner.

14. The transgenic non-human animal of claim 1, wherein said promoter is selected from the group consisting of beta-lactoglobulin promoter, Rb promoter, preproendothelin-1 promoter, beta-actin promoter, TetO promoter, metallothionein promoter, whey acidic protein (WAP) promoter, casein promoter and lactalbumin promoter.

15. The transgenic non-human mammal of claim 9, wherein said heparanase is expressed in, and secreted by, cells of mammary glands of said mammal.

16. The transgenic avian of claim 10, wherein said promoter is selected from the group consisting of chicken lyzozyme promoter, cytomegalovirus promoter and chicken immunoglobulin promoter.

17. The transgenic non-human avian of claim 10, wherein said heparanase is expressed in, and secreted by, egg producing cells of said avian.

18. Sex cells derived from the transgenic non-human animal of claim 1.

19. Semen derived from the transgenic non-human animal of claim 1.

20. An embryo derived from the transgenic non-human animal of claim 1.

21. A composition of matter comprising milk derived from a non-human transgenic mammal, said milk having detectable human heparanase activity.

22. A composition of matter comprising egg yolk and/or white from a transgenic avian, said egg yolk and/or white having detectable human heparanase activity.

23. A method of producing recombinant human heparanase, the method comprising the steps of:
(a) obtaining a transgenic non-human mammal having mammary glands, whose genome comprises an exogenous polynucleotide sequence integrated into said genome, said exogenous polynucleotide sequence including a promoter active in tissues of the non-human mammal, and a region encoding a human heparanase, wherein said promoter and said region encoding human heparanase are operably linked in said exogenous polynucleotide such that the recombinant human heparanase is secreted into milk being produced by said mammary glands;
(b) milking said non-human mammal so as to obtain milk containing the recombinant human heparanase; and
(c) purifying the recombinant human heparanase from said milk.

24. The method of claim 23, wherein said promoter active in tissues of said non-human mammal is a milk protein gene promoter.

25. The method of claim 24, wherein said milk protein gene promoter is selected from the group consisting of beta-lactoglobulin promoter, Rb promoter, preproendothelin-1 promoter, whey acidic protein (WAP) promoter, casein promoter and lactalbumin promoter.

26. A method of producing recombinant human heparanase, the method comprising the steps of:
(a) obtaining a transgenic female avian having egg producing cells whose genome comprises an exogenous polynucleotide sequence integrated into said genome, said exogenous polynucleotide sequence including a promoter active in tissues of said transgenic female avian, and a region encoding a human heparanase, wherein said promoter and said region encoding human heparanase are operably linked in said exogenous polynucleotide such that the recombinant human heparanase is secreted into eggs being produced by said egg producing cells;
(b) collecting eggs laid by said transgenic female avian so as to obtain eggs containing the recombinant human heparanase; and
(c) purifying the recombinant human heparanase from said eggs.

27. The method of claim 26, wherein said promoter active in tissues of said transgenic female avian is an egg protein gene promoter.

28. The method of claim 27, wherein said egg protein gene promoter is selected from the group consisting of chicken lyzozyme promoter and chicken immunoglobulin promoter.
